(19) United States
(12) Patent Application Publication
(10) Pub. No.: US 2017/0269075 A1
(43) Pub. Date: Sep. 21, 2017

(54) BIOMARKERS PREDICTIVE OF LUPUS PROGRESSION AND USES THEREOF

(71) Applicants: Biogen MA Inc., Cambridge, MA (US); University of Maryland, Baltimore, Baltimore, MD (US); THE JOHNS HOPKINS UNIVERSITY, Baltimore, MD (US)

(72) Inventors: Ann Marie RANGER, Reading, MA (US); Jadwiga BIENKOWSKA, Cambridge, MA (US); Carrie Greene WAGER, Watertown, MA (US); Laurence Saul MAGDER, Timonium, MD (US); Michelle PETRI, Timonium, MD (US)

(21) Appl. No.: 15/310,204

(22) PCT Filed: May 11, 2015
(86) PCT No.: PCT/US2015/030190

§ 371 Date: Nov. 10, 2016

Related U.S. Application Data
(60) Provisional application No. 61/991,939, filed on May 12, 2014.
(60) Provisional application No. 62/079,813, filed on Nov. 14, 2014.

(51) Int. Cl. G01N 33/564; G06F 19/24; G06F 19/00; C12Q 1/68

(52) U.S. Cl. CPC G01N 33/564; C12Q 1/6883; G06F 19/24; G06F 19/3437; G06F 19/345; G01N 2333/70575; C12Q 2600/158; G01N 2800/104; C12Q 2600/106; G01N 2800/52; C12Q 2600/118

(57) ABSTRACT

Methods, systems and kits to detect and/or quantify lupus and disease progression in a subject having, or at risk of having, lupus are disclosed.

### RELATED APPLICATIONS

This application claims the benefit of U.S. Provisional Application No. 61/991,939, filed May 12, 2014, and U.S. Provisional Application No. 62/079,813, filed Nov. 14, 2014, the contents of both of which are incorporated herein by reference in their entirety.

### BACKGROUND OF THE INVENTION

Lupus is a group of conditions with similar underlying mechanisms involving autoimmunity. In these conditions, antibodies created by the body to attack antigens (e.g., viruses, bacteria) become unable to differentiate between antigens and healthy tissue. Thus, these antibodies begin to attack the body's own healthy tissues. Triggers for lupus include viruses, bacteria, allergens (both IgE and hypersensitivity), hormones (e.g., estrogens), environmental stimulants (e.g., ultraviolet light, sunlight, stress, smoking, trauma, scratching, burn, coldness), and certain medications.

Lupus is generally a chronic disease in which the signs and symptoms tend to come and go. Common signs or symptoms of lupus include, for example, joint pain and stiffness; muscle aches, pains, or weakness, fever with no known cause; feeling very tired; butterfly-shaped rash across the nose and cheeks; other skin rashes; unusual weight loss or weight gain; anemia; trouble thinking, memory problems, and confusion; kidney problems with no known cause; chest pain when taking a deep breath; sun or light sensitivity; hair loss; and purple or pale fingers or toes from cold or stress. Less common symptoms include blood clots, seizures, strokes, sores in the mouth or nose, severe headache, dizzy spells, “seeing things” or not being able to judge reality, feeling sad, and dry or irritated eyes. Lupus also increases the risk of developing various other diseases and/or causes other diseases to occur earlier in life. Such diseases include heart disease, osteoporosis, and kidney disease. See e.g., Lupus Fact Sheet by the U.S. Department of Health and Human Services, Office on Women's Health (womenshealth.gov/publications/our-publications/fact-sheet/lupus.cfm; accessed Jun. 18, 2012).

Types of lupus include, for example, systemic lupus erythematosus (SLE), cutaneous lupus erythematosus (CLE) (CLE includes, e.g., acute cutaneous lupus erythematosus (ACLE), subacute cutaneous lupus erythematosus (SCLE), intermittent cutaneous lupus erythematosus, and chronic cutaneous lupus), drug-induced lupus, and neonatal lupus. About 70% of all cases of lupus are SLE. CLE can have symptoms that are limited to the skin or can be seen in those with SLE.

Lupus affects predominately young women. More than 90% of people with lupus are women between the ages of 15 and 45. African-American, Latina, Asian, and Native American women are at greater risk of developing lupus than are white women. Men are at higher risk for developing lupus before puberty and after age 50. For African-American women between the ages of 15 and 64, the prevalence is one per 245 women. This prevalence rate for African-American women makes lupus one of the most common chronic diseases of this population. See, e.g., lupus.org/webmodules/webarticlesne themplates/new_empty.aspx?articleid=413&zoneid=99, accessed Jun. 18, 2012.

Diagnosing and monitoring of lupus remain problematic. Thus, the need exists for novel ways of identifying, assessing and treating individuals affected by the disease.

### SUMMARY OF THE INVENTION

The present invention provides, at least in part, methods, systems and kits for the identification, assessment and/or treatment of a subject having lupus, e.g., SLE. In one embodiment, the methods and kits include the step of detecting and/or quantifying disease progression in the subject (e.g., a subject with lupus). In one embodiment, Applicants evaluated the relationship between six transcriptional signatures or proteins, which were independently predictive of future disease activity in subjects with SLE. It was discovered, at least in part, that after controlling for sex, race, and other biomarkers, only a subset of the transcriptional signatures or biomarkers was independently predictive of long-term lupus disease progression, e.g., over a 1 year period.

In certain embodiments, the methods, systems and kits disclosed herein include a determination of a value of disease status or progression (also referred to individually herein as “disease status value” or “disease progression value,” respectively) that includes a measure of one, two, three or all of a BAFF biomarker, a TWEAK biomarker, a neutrophil gene signature or an interferon (IFN) signature. The methods and kits disclosed herein can be used, for example, for one or more of: (i) diagnosing, prognosing and/or evaluating, a subject (e.g., a subject having lupus); (ii) evaluating responsiveness to, or monitoring, a therapy (e.g., a lupus therapy); (iii) to stratify a subject (e.g., a lupus patient or patient population) as being disease progressing; and/or (iv) more effectively monitoring, treating lupus, or preventing worsening of disease progression and/or flares.

In one aspect of the invention, Applicants have discovered that conventional usage of various biomarkers and/or indicators to detect, quantify, diagnose, prognose, and/or treat lupus progression in a subject (e.g., a lupus patient) generates inconsistent results, for example, when employed to predict future disease activity. Accordingly, various aspects and embodiments disclosed herein are directed to increasing a confidence level and/or accuracy associated with the generation of disease status or progression values used to evaluate the subject. In one embodiment, a multivariate characterization model was used to increase the confidence level associated with the disease progression value. For example, the multivariate characterization model can include a combination of two, three or all of a BAFF biomarker, a TWEAK biomarker, a neutrophil gene signature, and an interferon signature (e.g., IFN-alpha or IFN-gamma signature). The multivariate characterization model can be used in methods, systems, and kits for the identification, assessment and/or treatment of a subject having lupus, and further used to established confidence values associated with assessment and/or treatment information associated with the subject.

According to one embodiment, the multivariate characterization model incorporates at least two independent indicators (e.g., a BAFF biomarker, UTWEAK (urinary TWEAK) biomarker, interferon (IFN) signature (e.g., IFN-alpha or IFN-gamma signature) and/or neutrophil gene signature, among other options). The relationship modeling between biomarker(s), indicator(s) (e.g., gene signature), etc., can be used to identify independent predictors of disease activity. In further embodiments, relationship modeling can be employed to control for age, race, sex, and candidate markers and/or candidate indicators. In one example, relationship modeling of one or more transcription signatures and one or more biomarkers with respect to lupus disease activity long-term, e.g., over a year period, can be employed to eliminate markers and signatures that are individually associated with disease activity but are not significant under control for any one or more of: sex, race, and other markers/signatures. Once individually associated markers and/or indicators are eliminated, the remaining independent markers/indicators can be used to improve assessments of disease activity. In some examples, relationship modeling establishes independent and additive predictors (e.g., combination increases confidence) of disease activity that can be combined into a multivariate model for patient evaluation.

In some embodiments, each relationship of independent indicators (e.g., a BAFF biomarker, UTWEAK (urinary TWEAK) biomarker, interferon (IFN) signature (e.g., IFN-α or IFN-γ signature) and/or neutrophil gene signature can be associated with a confidence score for the association (e.g., IFN-alpha increase can be associated with a p value of 0.0002; neutrophil gene signature increase is associated with a p value of 0.0090; and TWEAK protein increase is associated with a p value 0.0006). Other examples include correlations between biomarker measurements and increases in a renal component of a SLEDAI score (e.g., for patients in the top 50% of IFN-Alpha a one standard deviation increase in IFN-Alpha is associated with a 0.33 point higher mean renal component of SLEDAI; on average high neutrophil signature is associated with 0.39 higher mean renal component of SLEDAI; for patients with high neutrophil signature a one standard deviation increase in TWEAK is associated with a 0.57 increase in a mean renal component of SLEDAI). In some embodiments, the relationships can also be associated with a confidence score for the association (e.g., IFN-Alpha increase in renal SLEDAI can be associated with a p value of 0.024; neutrophil gene indicator is associated with a p value of 0.010; and high neutrophil signature, the increase in TWEAK/renal SLEDAI score is associated with a p value 0.0001).

In further embodiments, users of the methods and kits for identification, assessment and/or treatment of a subject having lupus, can evaluate assessment and/or treatment information based on indications of associated confidence levels. In still further embodiments, various methods, systems and kits can include user interfaces that enable users of the methods and kits to define threshold confidence levels for assessment and/or treatment information. The user interfaces can be configured to provide recommendations on one or more tests that may be used to achieve an input confidence level.

Accordingly, in one aspect, the invention features a method of treating or preventing lupus in a subject. The method includes:

acquiring a value of disease progression that comprises a measure of one, two, three or all of: a BAFF biomarker, a TWEAK biomarker, a neutrophil gene signature, and/or an interferon gene signature (e.g., IFN-alpha or IFN-gamma signature), and

responsive to a determination of the value of disease progression, performing one, two, three, four or more of:

identifying the subject as disease progressing;

administering a lupus therapy (e.g., a first or second lupus therapy);

selecting or altering a dosing of a lupus therapy;

selecting or altering the schedule or time course of a lupus therapy; or

selecting an alternative (e.g., second) lupus therapy, thereby treating or preventing lupus in the subject.

In a related aspect, the invention features a method of evaluating a subject having, or at risk of having lupus. The method includes acquiring a value of disease progression that comprises a measure of one, two, three or all of: a BAFF biomarker, a TWEAK biomarker, a neutrophil gene signature, and/or an interferon gene signature (e.g., IFN-alpha or IFN-gamma signature), thereby evaluating the subject.

In yet another aspect, the invention features a method of evaluating or monitoring the effectiveness of a therapy (e.g., a lupus therapy) in a subject having lupus. The method includes:

acquiring a value of disease progression that comprises a measure of one, two, three or all of: a BAFF biomarker, a TWEAK biomarker, a neutrophil gene signature, and/or an interferon gene signature (e.g., IFN-alpha or IFN-gamma signature), thereby evaluating or monitoring the effectiveness of the therapy in the subject.

In a related aspect, a lupus therapy for use in a method of treating or preventing lupus in a subject is provided. The method includes:

acquiring a value of disease progression that comprises a measure of two, three or all four of: a TWEAK biomarker (e.g., urinary TWEAK (UTWEAK)), a neutrophil gene signature, a BAFF biomarker (e.g., BAFF mRNA), or an interferon (IFN) signature, and responsive to a determination of the value of disease progression:

administering a lupus therapy (e.g., a first or a second lupus therapy);

selecting or altering a dosing of a lupus therapy (e.g., a first or a second lupus therapy); or

selecting or altering the schedule or time course of a lupus therapy (e.g., a first or a second lupus therapy).

In one embodiment, the lupus therapy used in the method comprises one or more of: a nonsteroidal anti-inflammatory drug (NSAID); an antimalarial drug (e.g., hydroxychloroquine); a corticosteroid (e.g., a glucocorticoid); an immunosuppressant (e.g., azathioprine, mycophenolate mofetil, or methotrexate); an intravenous immunoglobulin; an anti-TWEAK antibody; an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; or an anti-BAFF antibody.

Any of the aforesaid methods can further include treating, or preventing in, a subject having lupus one or more symptoms associated with lupus. In certain embodiments, the treatment includes reducing, retarding, or preventing, a relapse, or the worsening of a disability, or the onset of sustained disease progression, in the subject (e.g., a subject having lupus). In one embodiment, the method includes, responsive to the value of disease status or progression, administering to the subject a lupus therapy (also referred to herein as a “lupus treatment”), in an amount sufficient to reduce one or more symptoms associated with lupus.

In certain embodiments, any of the aforesaid methods can further include one or more of the following:

(i) identifying the subject as being in need of a lupus therapy (e.g., a first lupus therapy, or a second or alternative lupus therapy);

(ii) identifying the subject as having an increased or a decreased response to a lupus therapy (e.g., a first lupus therapy, or a second or alternative lupus therapy);

(iii) identifying the subject as being stable or showing an improvement in one or more abilities or function, or showing a decline in one or more abilities or function;

(iv) diagnosing and/or prognosing the subject;

(v) selecting or altering the course of, a lupus therapy, a dose, a treatment schedule or time course, and/or the use of an alternative lupus therapy;

(vi) determining lupus disease progression in the subject;

(vii) administering a first lupus therapy, or a second or alternative lupus therapy to the subject; and/or

(viii) evaluating the effectiveness of a therapy in treating or preventing a progressive form of lupus,

wherein a change in the disease progression value relative to a specified or reference parameter indicates one or more of: identifies the subject as being in need of the first lupus therapy, or an additional or alternative lupus therapy; identifies the subject as having an increased or decreased response to the therapy; determines the treatment to be used; and/or determines or predicts the time course of the onset and/or progression of lupus.

In another aspect, the invention features a kit for evaluating a subject having, or at risk of having lupus (e.g., a lupus patient). The kit comprises:

a means or tests for evaluating one, two or all three of a BAFF biomarker, a TWEAK biomarker, a neutrophil gene signature, and/or an interferon signature (e.g., any of the biomarkers described herein) in the subject (e.g., a sample obtained from a subject); and

a means for determining a value of disease progression associated with the subject, prior to, during, and/or after a lupus therapy (e.g., the value of disease progression as described herein).

In another aspect, the invention features a system for evaluating lupus in a subject. The system includes:

at least one processor operatively connected to a memory, the at least one processor when executing is configured to:

acquire a value of disease progression that comprises a measure of a neutrophil gene signature or an interferon signature (e.g., IFN-alpha or IFN-gamma signature) and one or both of: a BAFF biomarker (e.g., BAFF mRNA) or a TWEAK biomarker (e.g., UTWEAK), and

responsive to a determination of the value of disease progression, perform one, two, three, four or more of:

identify the subject as disease progressing;

recommend a lupus therapy;

recommend a selection or alteration of
(i) a dosing of a lupus therapy;
(ii) a schedule or time course of a lupus therapy; or
(iii) an alternative lupus therapy.

In certain embodiments, the system includes:

at least one processor operatively connected to a memory, the at least one processor when executing is configured to
accept patient test information on one or more indicators for Lupus disease activity;
evaluate at least one, two, or all, independent indicators of disease progression (e.g., BAFF biomarker, neutrophil signature, an interferon signature (e.g., IFN-alpha or IFN-gamma signature), a TWEAK biomarker);
determine a confidence level associated with a prognoses of disease progression based on a multivariate model applied to the at least one, two, or all, independent indicators of disease progression; and
display the confidence level associated with the prognoses of disease progression.

In another aspect, the invention features a system for evaluating lupus in a subject. The system includes at least one processor operatively connected to a memory, the at least one processor when executing is configured to:

acquire a value of disease progression that comprises a measure of a first marker including one, two, or three of neutrophil gene signature, IFN-Alpha, and TWEAK, and additional markers comprising one, two, or more additional markers including one, two, or more of neutrophil gene signature, IFN-Alpha, and TWEAK, to score disease activity;

responsive to a determination of the value of disease progression, perform one, two, three, four or more of:
identify the subject as disease progressing;
recommend a lupus therapy; and
recommend a selection or alteration of:
   a dosing of a lupus therapy;
   a schedule or time course of a lupus therapy; or
   an alternative lupus therapy.

According to another aspect, the invention features a system for evaluating lupus in a subject. The system includes at least one processor operatively connected to a memory, the at least one processor when executing is configured to:

accept patient test information on one or more indicators for Lupus disease activity;

evaluate at least one, two, three, or all, available indicators of disease progression (e.g., IFN-Alpha, neutrophil gene signature, TWEAK, BAFF, and IFN-Gamma);

determine a confidence level associated with a prognoses of disease progression based on a multivariate model applied to the at least one, two, three, or all, available indicators of disease progression; and

display the confidence level associated with the prognoses of disease progression.

In certain embodiments, any of the aforesaid systems can further include at least one processor further configured to select indicators of disease progression to generate the multivariate model responsive to identification of independent indicators of disease progression.

According to one aspect, the invention features a system for evaluating lupus in a subject. The system includes at least one processor operatively connected to a memory, the at least one processor when executing is configured to:

accept patient test information on one or more indicators for lupus disease activity;

evaluate at least one, two, three, or all, indicators of disease progression (e.g., IFN-Alpha, neutrophil gene signature, TWEAK, BAFF, and IFN-Gamma);

identify independent indicators of disease progression from the at least one, two, three, or all of indicators of disease progression;

determine a confidence level associated with a prognoses of disease progression based on a multivariate model applied to the at least one, two, three, or all, available indicators of disease progression; and display the confidence level associated with the prognoses of disease progression.

In certain embodiments, any of the aforesaid systems can further specify one or more of the following:

(i) wherein the at least one processor is further configured to select the multivariate model to apply to the at least one, two, three, or all, available indicators of disease progression responsive to the confidence level associated with the identified independent indicators of disease progression;

(ii) wherein the at least one processor is further configured to optimize the determination of the prognoses of disease progression responsive to maximizing the confidence level associated with the independent indicators of disease progression; and

(iii) wherein the at least one processor is further configured to optimize the determination of the prognoses of disease progression responsive to substituting highly correlated indicators (e.g., BAFF and IFN-Alpha, IFN-Alpha and IFN-Gamma) where the corresponding independent indicator is unavailable or suspect.

Additional embodiments or features of any of the foregoing methods, systems and kits disclosed herein include one or more of the following:

### Biomarkers and Disease Progression

In certain embodiments, Applicants discovered that certain biomarkers and/or signatures having observed associations for lupus disease progression or activity may include spurious associations, for example, based on confounding by correlation with other biomarkers or patient characteristics, e.g., race, gender, other biomarkers. Accordingly, in certain embodiments, various biomarkers and/or signatures were evaluated to determine which ones have independent associations that can be used in methods and kits for the identification, assessment and/or treatment of a subject having lupus.

In one embodiment, example independent biomarkers and/or indicators include, for example, BAFF mRNA (e.g., measured in serum), a neutrophil signature, an interferon signature, and urinary TWEAK (also referred to as “UTWEAK”).

In certain embodiments, an increase of about 1 standard deviation relative to a reference value (e.g., a median value for a lupus patient population) in BAFF is indicative of an increase in a range of from about 0.05 to about 0.45 (e.g., 0.09 to 0.40, 0.15 to 0.35, 0.2 to 0.3, or 0.25-0.26), of a mean SLEDAI score. In one example, the correlation between the increase in BAFF and the increase in SLEDAI score can be associated with a probability that the correlation is incorrect. For example, a p-value can be used to evaluate the determined relationship, wherein the p-value is the probability of incorrectly obtaining the test result (i.e., assuming that there is no correlation the probability of obtaining the same or better results). In one example, a 1 standard deviation increase in BAFF is indicative of an increase in SLEDAI score with a p-value failing in the range of less than 1% (including, for example, p=0.0034).

In other embodiments, a measure of the neutrophil gene signature being in the top 15% in a lupus patient population (e.g., of a median value for a lupus patient population) is indicative of a increase of from about 0.15 to about 1.5 (e.g., 0.2 to 1.15, 0.3 to 1, 0.4 to 0.9, 0.5-0.8, 0.6 to 0.7, or about 0.66), of a mean SLEDAI score. In some examples, the relationship between neutrophil gene signature and SLEDAI can be established with a p-value falling in the range of less than one percent (including, for example, p=0.0056).

In other embodiments, a measure of the urinary TWEAK biomarker (e.g., normalized TWEAK protein detected, for example, by ELISA) being in the top 15% in a lupus patient population (e.g., of a median value for a lupus patient population) is indicative of an increase of from about 0.20 to about 0.95 (e.g., 0.25 to 0.90, 0.3 to 0.8, 0.4 to 0.7, 0.5-0.6, or about 0.58), of the mean SLEDAI score. In some examples, the relationship between neutrophil gene signature and SLEDAI can be established with a p-value falling in the range of less than one tenth of a percent (including, for example, p=0.0006).

In further embodiments, a patient evaluation can be based on renal disease activity and additional biomarker and/or indicators can be employed to determine, predict, and/or assess renal disease activity. For example, an increase of about 1 standard deviation relative to a reference value (e.g., a median value for a lupus patient population) in the IFN signature is indicative of an increase of a range of from about 0.05 to about 0.25 (e.g., 0.05 to 0.20, 0.08 to 0.15, 0.1 to 0.12, or about 0.11) of a mean renal SLEDAI score. When evaluating renal disease activity, the relationship between IFN signature and renal SLEDAI can be established with a p-value falling in the range of less than five percent (including, for example, p=0.04).

In other embodiments, various signatures/indicators remain significant for overall disease activity even with a focus on renal disease activity. For example, the neutrophil signature remained significant at similar levels as discussed more generically above with respect to SLEDAI versus renal SLEDAI. In another example, an increase of about 1 standard deviation relative to a reference value (e.g., a median value for a lupus patient population) in the urinary TWEAK biomarker is indicative of an increase of from 0.05 to about 0.45 (e.g., 0.09 to 0.40, 0.15 to 0.35, 0.2 to 0.3, or 0.25-0.26), of the mean renal SLEDAI score. When evaluating renal disease activity, the relationship between UTWEAK and renal SLEDAI can be established with a p-value falling in the range of less than one of a percent (including, for example, p<0.0001).

According to another aspect, having determined biomarkers and/or indicators that are independent, various methods and kits can increase a confidence level associated with detecting and/or quantifying disease progression in the subject (e.g., a subject with lupus), by combining the indicators and/or biomarkers.

Various combinations of biomarker and/or indicators can be used in various embodiments. For examples, methods and kits for detecting and/or quantifying disease progression in the subject can combine analysis of BAFF with one or more of Neutrophil and UTWEAK to characterize disease status or progression. In one example, disease status is characterized using BAFF and Neutrophil. In another example, BAFF and UTWEAK are analyzed to determine disease status. In further examples, neutrophil can be analyzed in conjunction with one or more of BAFF and UTWEAK. In yet others, UTWEAK can be analyzed in conjunction with one or more of BAFF and Neutrophil.

According to one embodiment, combinations that include at least one gene signature and at least one biomarker have increased confidence levels as predictive of future disease activity, relative to individual indicators as predictive of future disease activity. In some embodiments, methods and kits for identification, assessment and/or treatment of a subject having lupus can deliver confidence levels associated with respective indicators for disease activity. In further embodiments, users of the methods and kits can evaluate assessment and/or treatment information based on the confidence levels. In still further embodiments, various methods and kits can include user interfaces that enable users of the methods and kits to define threshold confidence levels for assessment and/or treatment information

In certain embodiments, the value of disease progression comprises a measure of a combination of two or all of: an interferon signature (e.g., IFN-alpha signature), a neutrophil gene signature, or a TWEAK biomarker (e.g., urinary TWEAK or UTWEAK).

In certain embodiments, the value of disease progression comprises a measure of a neutrophil gene signature, and one or both of an interferon signature (e.g., IFN-alpha signature) or a TWEAK biomarker (e.g., urinary TWEAK or UTWEAK).

In certain embodiments, the value of disease progression comprises a measure of an interferon signature (e.g., IFN-alpha signature), and one or both of a neutrophil gene signature or a TWEAK biomarker (e.g., urinary TWEAK or UTWEAK).

In certain embodiments, the value of disease progression comprises a measure of a TWEAK biomarker (e.g., urinary TWEAK or UTWEAK), and one or both of an interferon signature (e.g., IFN-alpha signature) or a neutrophil gene signature.

In certain embodiments, the value of disease progression comprises a measure of a combination of two of: a BAFF biomarker (e.g., BAFF mRNA), a TWEAK biomarker (e.g., urinary TWEAK or UTWEAK), or a neutrophil gene signature.

In other embodiments, the value of disease progression comprises a measure of a combination of two of: a BAFF biomarker (e.g., BAFF mRNA), a TWEAK biomarker (e.g., UTWEAK), or a neutrophil gene signature.

In other embodiments, the value of disease progression comprises a measure of a combination of all three of: a BAFF biomarker (e.g., BAFF mRNA), a TWEAK biomarker (e.g., UTWEAK), or a neutrophil gene signature.

In other embodiments, the value of disease progression comprises a measure of a neutrophil gene signature and one or both of: a BAFF biomarker (e.g., BAFF mRNA) or a TWEAK biomarker (e.g., UTWEAK).

In other embodiments, the value of disease progression comprises a measure of an IFN signature. In one embodiment, the interferon signature provides an indication of renal disease activity. In one embodiment, the interferon signature in an interferon-alpha gene signature. In other embodiments, the interferon signature in an interferon-gamma gene signature.

The IFN signature (e.g., a Type I Interferon signature) can be used according to signatures known in the art, e.g., as described in Thurlings, et al. (2010) Arthritis and Rheumatology Vol. 62(12):3607-3614; Hall, J. C. et al. (2012) PNAS Vol. 109(43):17609-17614).

In other embodiments, methods, kits and systems further comprising determining a confidence level associated with the value of disease progression. In one embodiment, the confidence level is increased as the number of measures of biomarker and signature increases.

In other embodiments, the value of disease progression is associated with a multivariate model.

In certain embodiments, an increase in the measure of the BAFF biomarker (e.g., BAFF mRNA) of about 1 standard deviation (SD) relative to reference value (e.g., a median value for a lupus patient population) is indicative of an increase in a range of from about 0.05 to about 0.45 (e.g., 0.09 to 0.40, 0.15 to 0.35, 0.2 to 0.3, or 0.25-0.26), of the SLEDAI score (e.g., a mean SLEDAI score).

In other embodiments, a measure of the TWEAK biomarker (e.g., normalized TWEAK protein) being in the top 15% in a lupus patient population (e.g., of a median value for a lupus patient population) is indicative of a mean average increase of from about 0.20 to about 0.95 (e.g., 0.25 to 0.90, 0.3 to 0.8, 0.4 to 0.7, or 0.5-0.6), of the SLEDAI score (e.g., a mean SLEDAI score).

In yet other embodiments, an increase of the TWEAK biomarker of about 1 standard deviation (SD) relative to reference value (about 0.22 μg of TWEAK/mg of creatinine) is indicative of an increase in a range of from about 0.20 to about 0.95 (e.g., 0.25 to 0.90, 0.3 to 0.8, 0.4 to 0.7, or 0.5-0.6), of the SLEDAI score (e.g., a mean SLEDAI score).

In other embodiments, a measure of the neutrophil gene signature being in the top 15% in a lupus patient population (e.g., of a median value for a lupus patient population) is indicative of a mean average increase of from about 0.15 to about 1.5 (e.g., 0.2 to 1.15, 0.3 to 1, 0.4 to 0.9, 0.5-0.8, or 0.6 to 0.7), of the SLEDAI score (e.g., a mean SLEDAI score).

In other embodiments, an increased measure of the neutrophil gene signature is indicative of a mean average increase of from about 0.1 to about 1 (e.g., 0.2 to 0.8, 0.3 to 0.5, 0.3 to 0.4, or 0.39), of the renal SLEDAI score (e.g., a mean renal SLEDAI score).

In other embodiments, an increase in the measure of the IFN signature (e.g., IFN-α signature) being in the top 55% in a lupus patient population (e.g., a median value for a lupus patient population) of about 1 standard deviation (SD) relative to reference value (e.g., a median value for a lupus patient population) is indicative of an increase in a range of from about 0.2 to 1.15, 0.3 to 1, 0.4 to 0.9, 0.5-0.8, or 0.6 to 0.7 (e.g., about 0.61) of the SLEDAI score (e.g., mean SLEDAI score).

In other embodiments, an increase in the measure of the IFN signature (e.g., IFN-α signature) being in the top 50% in a lupus patient population (e.g., a median value for a lupus patient population) of about 1 standard deviation (SD) relative to reference value (e.g., a median value for a lupus patient population) is indicative of an increase in a range of from about 0.1 to 1, 0.2 to 0.8, 0.3 to 0.6, 0.3-0.4, or about 0.33 of the renal SLEDAI score (e.g., mean renal SLEDAI score).

In other embodiments, an increase in the measure of the IFN signature of about 1 standard deviation (SD) relative to reference value (e.g., a median value for a lupus patient population) is indicative of an increase in a range of from about 0.01 to about 0.30, 0.05 to 0.20, 0.08 to 0.15, 0.1 to 0.12, or about 0.11 of the renal SLEDAI score (e.g., mean renal SLEDAI score).

In yet other embodiments, an increase in the measure of the TWEAK biomarker of about 1 standard deviation (SD) relative to reference value (e.g., a median value for a lupus patient population) in the measure of the TWEAK biomarker (e.g., urinary TWEAK) is indicative of an increase in a range of from about 0.10 to about 0.35, 0.15 to 0.3, 0.20 to 0.26, or about 0.25 of the renal SLEDAI score (e.g., mean renal SLEDAI score).

In yet other embodiments, for those subjects having a high level of a neutrophil signature, an increase in the measure of the TWEAK biomarker of about 1 standard deviation (SD) relative to reference value (e.g., a median value for a lupus patient population) in the measure of the TWEAK biomarker (e.g., urinary TWEAK) is indicative of an increase in a range of from about 0.1 to about 1, 0.3 to 0.7, 0.4 to 0.6, or about 0.57 of the renal SLEDAI score (e.g., mean renal SLEDAI score).

In certain embodiments, the SLEDAI score change (e.g., increase) is acquired at least three, four, five, or six months or 1 or 2 years after the value of disease progression is acquired. In one embodiment, the SLEDAI scale comprises the SELENA SLEDAI scale. In certain embodiments, a SLEDAI score of between 1-5 is indicative of mild disease activity in the subject; a SLEDAI score of between 6-10 is indicative of moderate disease activity in the subject; a SLEDAI score of between 11-19 is indicative of high disease activity in the subject; and a SLEDAI score of 20-105 is indicative of very high disease activity in the subject.

In certain embodiments, the disease progression in the lupus subject comprises a steady worsening of one or more symptoms over time. The symptoms of lupus can include one or more of: seizure, psychosis, organic brain syndrome, visual disturbance, cranial nerve disorder, lupus headache, cerebrovascular accident, vasculitis, arthritis, myositis, urinary casts, hematuria, proteinuria, pyuria, rash, alopecia, mucosal ulcers, pleurisy, pericarditis, low complement, increased DNA binding, fever, thrombocytopenia, and/or leukopenia.

In certain embodiments, the reference parameter is obtained from one or more of: a baseline or prior value for the subject, the subject at a different time interval, an average or median value for a lupus patient population, a healthy control, or a healthy subject population. In one embodiment, the reference parameter is the median value for a lupus patient population.

In other embodiments, the measure of the BAFF biomarker comprises a value for expression of the gene, e.g., a BAFF gene product (e.g., a BAFF nucleic acid or polypeptide).

In other embodiments, the measure of the neutrophil gene signature comprises a value for expression of at least two, three, four, five, six, seven, eight, nine, ten, eleven, twelve, fifteen or more genes comprising a neutrophil gene signature. For example, the gene in the neutrophil gene signature can be chosen from at least two, three, four, five, six, seven, or all eight BPI, CEACAM6, CEACAM8, DEFA4, LCN2, LTF, MMP8, and OLFM4.

In certain embodiments, the value for expression of the gene comprises a value for a transcriptional parameter, e.g., the level of a gene product (e.g., an mRNA or gene transcript). In one embodiment, the level of a BAFF gene product (e.g., a BAFF mRNA or BAFF gene transcript) is detected or acquired. In other embodiments, the level of a gene product (e.g., an mRNA or gene transcript) of a neutrophil gene signature, e.g., at least one, two, three, four, five, six, seven, or all eight of BPI, CEACAM6, CEACAM8, DEFA4, LCN2, LTF, MMP8, and/or OLFM4, is detected or acquired.

In other embodiments, the measure of a TWEAK biomarker comprises a value for expression of the gene, e.g., a TWEAK gene product (e.g., a TWEAK nucleic acid or polypeptide). In one embodiment, the TWEAK biomarker is a TWEAK polypeptide, e.g., a soluble TWEAK polypeptide. In certain embodiments, the level of the TWEAK polypeptide, e.g., a soluble TWEAK polypeptide is detected or acquired from a urine sample.

In certain embodiments, the biomarker evaluated is a gene or gene product, e.g., cDNA, RNA (e.g., mRNA or gene transcript), or a polypeptide. In embodiments where the biomarker is a polypeptide, the polypeptide can be detected, or the level determined, by any means of polypeptide detection, or detection of the expression level of the polypeptides. For example, the polypeptide can be detected using a reagent which specifically binds with the biomarker polypeptides. In another embodiment, the reagent is selected from the group consisting of an antibody, an antibody derivative, and an antibody fragment. In one embodiment, the biomarker is detected using antibody-based detection techniques, such as enzyme-based immunoabsorbent assay, immunofluorescence cell sorting (FACS), immunohistochemistry, immunofluorescence (IF), antigen retrieval and/or microarray detection methods. In one embodiment, the detection, or determination of the level, of the biomarker includes contacting the sample with a reagent, e.g., an antibody that binds to the biomarker and detecting or determining the level of the reagent, e.g., the antibody, bound to the biomarker. The reagent, e.g., the antibody, can be labeled with a detectable label (e.g., a fluorescent or a radioactive label). Polypeptide detection methods can be performed in any other assay format, including but not limited to, ELISA, RIA, and mass spectrometry. The amount, structure and/or activity of the biomarker polypeptides can be compared to a reference value, e.g., a control sample, or a pre-determined value. In one embodiment, the detection or determination step includes a multiplex bead enzyme-based immunoabsorbent assay. In such embodiments, the detection is usually driven by a flourescent molecule bound to the detection antibody by biotin.

In other embodiments where the biomarker is a nucleic acid, the nucleic acid can be detected, or the level determined, by any means of nucleic acid detection, or detection of the expression level of the nucleic acids, including but not limited to, nucleic acid hybridization assay, amplification-based assays (e.g., polymerase chain reaction), sequencing, screening analysis (including metaphase cytogenetic analysis by standard karyotype methods, FISH, spectral karyotyping or MFISH, and comparative genomic hybridization), and/or in situ hybridization. The amount, structure and/or activity of the one or more lupus biomarker nucleic acid (e.g., DNA or RNA) can be compared to a reference value or sample, e.g., a control sample, or a pre-determined value.

In certain embodiments, the measure of biomarker and/or signature is acquired before, at the same time, or during the course of a lupus therapy.

In other embodiments, the measure of biomarker and/or signature is acquired at least one, two, three, four, five, six months, 1 or 2 years, or longer after the initiation of a lupus therapy.

In other embodiments, the SLEDAI score is acquired at least three, four, five, or six months or 1 or 2 years after the value of disease progression is acquired.

In yet other embodiments, the SLEDAI score is acquired at least 1 or 2 years after the value of disease progression is acquired.

In yet another embodiment, the one or more biomarkers are assessed at pre-determined intervals, e.g., a first point in time and at least at a subsequent point in time. In one embodiment, a time course is measured by determining the time between significant events in the course of a patient's disease, wherein the measurement is predictive of whether a patient has a long time course. In another embodiment, the significant event is the progression from primary diagnosis to death. In another embodiment, the significant event is the progression from primary diagnosis to worsening disease. In another embodiment, the significant event is the progression from primary diagnosis to relapse. In another embodiment, the significant event is the progression to death. In another embodiment, the significant event is the progression from remission to relapse. In another embodiment, the significant event is the progression from relapse to death. In certain embodiments, the time course is measured with respect to one or more overall survival rate, time to progression and/or using the SLEDAI score or other assessment criteria.

### Lupus Therapy

The methods described herein can further include treating, or preventing in, a subject (e.g., a subject having lupus one or more symptoms associated with lupus). For example, a lupus therapy can be administered to the subject, in an amount sufficient to reduce one or more symptoms associated with lupus. In certain embodiments, the treatment or prevention comprises reducing, retarding or preventing, a flare, or the worsening of the disease, in the lupus subject.

In certain embodiments, the lupus comprises systemic lupus erythematosus (SLE) (e.g., lupus nephritis), cutaneous lupus erythematosus (CLE) (e.g., acute cutaneous lupus erythematosus (ACLE), subacute cutaneous lupus erythematosus (SCLE), intermittent cutaneous lupus erythematosus, and chronic cutaneous lupus), drug-induced lupus, and/or neonatal lupus. In one embodiment, the lupus is SLE.

In certain embodiments, the lupus therapy comprises one or more of: a nonsteroidal anti-inflammatory drug (NSAID); an antimalarial, including, for example, hydroxychloroquine; a corticosteroid, including, for example, a glucocorticoid; an immunosuppressant, including, for example, azathioprine, mycophenolate mofetil, or methotrexate; an intravenous immunoglobulin; an anti-TWEAK antibody; an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; or an anti-BAFF antibody.

In certain embodiments, the subject is administered, or is undergoing a first lupus therapy. Upon determination that the subject is less responsive or shows disease progression when treated with the first therapy, a second or an alternative therapy can be administered when a patient.

In certain embodiments, the lupus therapy comprises a first therapy chosen from one or more of:

(i) nonsteroidal anti-inflammatory drug (NSAID);

(ii) an antimalarial, including, for example, hydroxychloroquine;

(iii) a corticosteroid,

(iv) an immunosuppressant, including, for example, azathioprine, mycophenolate mofetil, or methotrexate; or

(v) an intravenous immunoglobulin.

In other embodiments, the lupus therapy comprises a second or alternative therapy chosen from one or more of

(i) an anti-TWEAK antibody;

(ii) an anti-CD40L antibody;

(iii) an anti-CD40 antibody;

(iv) an anti-CD20 antibody;

(v) an anti-interferon antibody;

(vi) rapamycin;

(vii) arsenic trioxide;

(viii) 5-chloro-N-ethyl-4-hydroxy-1-methyl-2-oxo-N-phenyl-1,2-dihydroquinoline-3-carboxamide;

(ix) an anti-BDCA2 antibody; or

(x) an anti-BAFF antibody.

### Samples

In certain embodiments of the methods, the methods further include obtaining a sample from the subject. In one embodiment, the sample is chosen from a non-cellular body fluid; or a cellular or tissue fraction. In certain embodiments, the method further includes the step of obtaining the sample, e.g., a biological sample, from the subject. In one embodiment, the method includes the step of obtaining a predominantly non-cellular fraction of a body fluid from the subject. The non-cellular fraction can be urine, plasma, serum, or other non-cellular body fluid. In one embodiment, the sample is a urine sample. In other embodiments, the body fluid from which the sample is obtained from an individual comprises blood, e.g., whole blood, or peripheral blood cells. In certain embodiments, the blood can be further processed to obtain plasma or serum. In another embodiment, the sample contains a tissue, cells (e.g., peripheral blood mononuclear cells (PBMC)).

A sample can include any material obtained and/or derived from a biological sample, including a polypeptide, and nucleic acid (e.g., genomic DNA, cDNA, RNA) purified or processed from the sample. Purification and/or processing of the sample can include one or more of extraction, concentration, antibody isolation, sorting, concentration, fixation, addition of reagents and the like, as described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### BRIEF DESCRIPTION OF THE FIGURES

FIG. 1 is a graph showing a smooth estimate of the mean modified SLEDAI as a function of normalized UTWEAK; and

FIG. 2 is a block diagram of a computer system on which various aspects and embodiments may be practiced.

FIGS. 3A and 3B are graphs showing BAFF gene transcript expression in SLE patients. FIG. 3A shows the distribution of BAFF gene transcript levels in SLE patients.

FIG. 3B shows the correlation of BAFF gene transcript levels with ModSLEDAI score (p<0.0001 for linear increase in mean ModSLEDAI).

FIGS. 4A and 4B are graphs showing IFN-alpha gene signature expression in SLE patients. FIG. 4A shows the distribution of IFN-alpha gene signature expression levels in SLE patients. FIG. 4B shows the correlation of IFN-alpha gene signature expression levels with ModSLEDAI score (p<0.0001 for linear increase in mean ModSLEDAI for values above 8.0).

FIGS. 5A and 5B are graphs showing IFN-gamma gene signature expression in SLE patients. FIG. 5A shows the distribution of IFN-gamma gene signature expression levels in SLE patients. FIG. 5B shows the correlation of IFN-gamma gene signature expression levels with ModSLEDAI score (p=0.011 for higher mean ModSLEDAI after for values above 7.25).

FIGS. 6A and 6B are graphs showing LDG-enriched neutrophil gene signature expression in SLE patients. FIG. 6A shows the distribution of LDG-enriched neutrophil gene signature expression levels in SLE patients. FIG. 6B shows the correlation of LDG-enriched neutrophil gene signature expression levels with ModSLEDAI score (p=0.0040 for higher mean ModSLEDAI for values above 7.0).

FIGS. 7A and 7B are graphs showing plasma cell gene signature expression in SLE patients. FIG. 7A shows the distribution of plasma cell gene signature expression levels in SLE patients. FIG. 7B shows the correlation of plasma cell gene signature expression levels with ModSLEDAI score (p=0.0039 for linear increase in mean ModSledai for values above 7.0).

FIGS. 8A and 8B are graphs showing TWEAK levels in SLE patients. FIG. 8A shows the distribution of TWEAK levels in SLE patients. FIG. 8B shows the correlation of TWEAK levels with ModSLEDAI score (p=0.0002 for increase for values above 0.22).

### DETAILED DESCRIPTION OF THE INVENTION

Lupus is an autoimmune disease that results in multi-organ involvement. This anti-self response in SLE patients is characterized by autoantibodies directed against a variety of nuclear and cytoplasmic cellular components. These autoantibodies bind to their respective antigens, forming immune complexes that circulate and eventually deposit in tissues. This immune complex deposition causes chronic inflammation and tissue damage.

Diagnosing and monitoring disease activity are problematic in patients with lupus. Diagnosis is problematic because the spectrum of disease is broad and ranges from subtle or vague symptoms to life-threatening multi-organ failure. There also are other diseases with multi-system involvement that can be mistaken for lupus, and vice versa. Criteria were developed for the purpose of disease classification in 1971 (Cohen, A S et al. (1971) Bull Rheum Dis 21:643-648) and revised in 1982 (Tan, E M et al. (1982) Arth Rheum 25:1271-1277) and 1997 (Hochberg, M C. (1997) Arth Rheum 40: 1725). These criteria are meant to ensure that patients from different geographic locations are comparable. Of the 11 criteria, the presence of four or more, either serially or simultaneously, is sufficient for classification of a patient as having lupus. Although the criteria serve as useful reminders of those features that distinguish lupus from other related autoimmune diseases, they are unavoidably fallible. Determining the presence or absence of the criteria often requires interpretation. If liberal standards are applied for determining the presence or absence of a sign or symptom, one could easily diagnose a patient as having lupus when in fact they do not. Similarly, the range of clinical manifestations in lupus is much greater than that described by the eleven criteria and each manifestation can vary in the level of activity and severity from one patient to another. To further complicate a difficult diagnosis, symptoms of lupus continually evolve over the course of the disease. New symptoms in previously unaffected organs can develop over time. There is no definitive test for lupus and, thus, it is often misdiagnosed.

Monitoring disease activity also is problematic in caring for patients with lupus. Lupus progresses in a series of flares, or periods of acute illness, followed by remissions. The symptoms of a flare, which vary considerably between patients and even within the same patient, include malaise, fever, symmetric joint pain, and photosensitivity (development of rashes after brief sun exposure). Other symptoms of lupus include hair loss, ulcers of mucous membranes and inflammation of the lining of the heart and lungs, which leads to chest pain. Red blood cells, platelets and white blood cells can be targeted in lupus, resulting in anemia and bleeding problems. More seriously, immune complex deposition and chronic inflammation in the blood vessels can lead to kidney involvement and occasionally kidney failure, requiring dialysis or kidney transplantation. Since the blood vessel is a major target of the autoimmune response in lupus, premature strokes and heart disease are not uncommon. Over time, however, these flares can lead to irreversible organ damage. In order to minimize such damage, earlier and more accurate detection of disease flares would not only expedite appropriate treatment, but would reduce the frequency of unnecessary interventions.

Two of the most commonly used instruments for lupus diagnosis are the Systemic Lupus Erythematosus Disease Activity Index (SLEDAI) (Bombardier, C et al. (1992) Arth Rheum 35: 63040), and the Systemic Lupus Activity Measure (SLAM) (Liang, M H et al. (1989) Arth Rheum 32: 1107-18). The SLEDAI includes 24 items, representing nine organ systems. The variables are obtained by history, physical examination and laboratory assessment. Each item is weighted from 1 to 8 based on the significance of the organ involved. For example, mouth ulcers are scored as 2, while seizures are scored as 8. The laboratory parameters that are included in the SLEDAI include white blood cell count, platelet count, urinalysis, serum C3, C4 and anti-dsDNA. The total maximum score is 105.

The SLAM includes 32 items representing 11 organ systems. The items are scored not only as present/absent, but graded on a scale of 1 to 3 based on severity. The total possible score for the SLAM is 86. Both the SLEDAI and the SLAM have been shown to be valid, reliable, and sensitive to change over time (Liang, M H et al.), and are widely used in research protocols and clinical trials. These indices are particularly useful for examining the value of newly proposed serologic or inflammatory markers of disease activity in lupus.

Despite the obvious utility of these instruments, there are some drawbacks. First, there is not always complete agreement between the SLAM and the SLEDAI in the same set of patients. There are several possible reasons for these discrepancies. Unlike the SLEDAI, the SLAM includes constitutional symptoms such as fatigue and fever, which may or may not be considered attributable to active lupus; this activity index relies on physician interpretation. In addition, the SLEDAI does not capture mild degrees of activity in some organ systems and does not have descriptors for several types of activity, such as hemolytic anemia. For these and other reasons, most studies incorporate more than one measure of disease activity. A general review of the state of the art can be found in Ramsey-Goldman, R. and Manzi, S. Systemic Lupus Erythematosus. In: Goldman and Hatch, Ed. Women and Health. Academic Press, San Diego, Calif. 2000: 704-723.

One of the most difficult challenges in clinical management of complex autoimmune diseases such as lupus is the accurate and early identification of the disease in a patient. In addition, no reliable diagnostic markers have been identified that enable clinicians or others to accurately define pathophysiological aspects of lupus, clinical activity, response to therapy, or prognosis.

Accordingly, methods, and kits for the identification, assessment and/or treatment of a subject having lupus are disclosed. In one embodiment, the methods and kits include the step of detecting and/or quantifying disease progression in the subject (e.g., a subject with lupus). In certain embodiments, the methods and kids include acquiring a value of disease status or progression (also referred to individually herein as “disease status value” or “disease progression value,” respectively, or by shorthand “disease status or progression value”). In one embodiment, the disease progression value includes a BAFF biomarker, a TWEAK biomarker, and/or a neutrophil gene signature. Thus, the methods and kits disclosed herein provide several advantages over existing methodologies, including, but not limited to, the ability to identify subjects as disease progressing.

Therefore, the invention can be used, for example, for one or more of: (i) diagnosing, prognosing and/or evaluating, a subject (e.g., a subject having lupus); (ii) evaluating responsiveness to, or monitoring, a therapy (e.g., a lupus therapy); (iv) to stratify a subject (e.g., a lupus patient or patient population) as being disease progressing; and/or (vi) more effectively monitoring, treating lupus, or preventing worsening of disease progression and/or flares.

Various aspects of the invention are described in further detail in the following subsections.

### Definitions

As used herein, each of the following terms has the meaning associated with it in this section.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

The term “altered level of expression” of a biomarker as described herein (e.g., BAFF, TWEAK, and/or a neutrophil gene signature) refers to an increase (or decrease) in the expression level of a marker in a test sample, such as a sample derived from a patient suffering from lupus that is greater or less than the standard error of the assay employed to assess expression. In embodiments, the alteration can be at least twice, at least twice three, at least twice four, at least twice five, or at least twice ten or more times greater than or less than the expression level of the biomarkers in a control sample (e.g., a sample from a healthy subject not having the associated disease), or the average expression level in several control samples. An “altered level of expression” can be determined at the protein or nucleic acid (e.g., mRNA) level.

A “biomarker” or “marker” is a gene, mRNA, or protein that undergoes alterations in expression that are associated with progression of lupus or responsiveness to treatment. The alteration can be in amount and/or activity in a biological sample (e.g., a blood, plasma, urine or a serum sample) obtained from a subject having lupus, as compared to its amount and/or activity, in a biological sample obtained from a baseline or prior value for the subject, the subject at a different time interval, an average or median value for a lupus patient population, a healthy control, or a healthy subject population (e.g., a control); such alterations in expression and/or activity are associated with progression of a disease state, such as lupus. For example, a marker of the invention which is associated with progression of lupus or predictive of responsiveness to therapeutics can have an altered expression level, protein level, or protein activity, in a biological sample obtained from a subject having, or suspected of having, lupus as compared to a biological sample obtained from a control subject.

A “nucleic acid” “marker” or “biomarker” is a nucleic acid (e.g., DNA, mRNA, cDNA) encoded by or corresponding to a marker as described herein. For example, such marker nucleic acid molecules include DNA (e.g., genomic DNA and cDNA) comprising the entire or a partial sequence of any of the nucleic acid sequences set forth, or the complement or hybridizing fragment of such a sequence. The marker nucleic acid molecules also include RNA comprising the entire or a partial sequence of any of the nucleic acid sequences set forth herein, or the complement of such a sequence, wherein all thymidine residues are replaced with uridine residues. A “marker protein” is a protein encoded by or corresponding to a marker of the invention. A marker protein comprises the entire or a partial sequence of a protein encoded by any of the sequences set forth herein, or a fragment thereof. The terms “protein” and “polypeptide” are used interchangeably herein.

The terms “homology” or “identity,” as used interchangeably herein, refer to sequence similarity between two polynucleotide sequences or between two polypeptide sequences, with identity being a more strict comparison. The phrases “percent identity or homology” and “% identity or homology” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value there between. Identity or similarity can be determined by comparing a position in each sequence that can be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences. The term “substantial homology,” as used herein, refers to homology of at least 50%, at least 60%, at least 65%, at least 70%, at least 75%, at least 80%, at least 85%, at least 90%, at least 95% or more.

As used herein, a “disease progression value” includes a measure (e.g., one or more measures) of a worsening, stability, or improvement of one or more symptoms and/or disability in a subject. In certain embodiments, disease progression is evaluated as a steady worsening, stability, or improvement of one or more symptoms and/or disability over time, as opposed to a relapse, which is relatively short in duration. In certain embodiments, the disease progression value is acquired in a subject with lupus (e.g., a subject with SLE, CLE, ACLE, SCLE, intermittent cutaneous lupus erythematosus, chronic cutaneous lupus, drug-induced lupus, or neonatal lupus).

As used herein, a “disease status value” includes a measure (e.g., one or more measures) of one or more symptoms and/or disability in a subject, e.g., an lupus subject as described herein. The term “disease status value” can include a disease progression value; however, it encompasses any status (e.g., worsening, stability or improvement) of a neurological disease in a subject, including, for example, steady worsening or relapse of lupus.

In certain embodiments, the disease status value includes a measure a BAFF biomarker, a TWEAK biomarker, and a neutrophil gene signature.

The disease status or progression value disclosed herein can be used as a means to confirm disease progression or non-progression in lupus patients. In certain embodiment, the disease progression value includes individual component parameters of one, two, three or all of the BAFF biomarker, TWEAK biomarker, an interferon signature or neutrophil gene signature assessments. In one embodiment, a disease progressor is a subject who possesses a disease progression value reflecting the following criteria:

a. BAFF: An increase in the measure of the BAFF biomarker (e.g., a BAFF mRNA) of about 1 standard deviation (SD) relative to reference value (e.g., a median value for a lupus patient population), confirmed at a second time point at least 3, 4, 5, or 6 months or 1-2 years apart by a mean average increase of from 0.2-0.3, or an increase in a range of from about 0.05 to about 0.45 (e.g., 0.09 to 0.40, 0.15 to 0.35, 0.2 to 0.3, or 0.25-0.26), of the SLEDAI score (e.g., a mean SLEDAI score);

b. TWEAK: the measure of the TWEAK biomarker being in the top 15% of measures of the TWEAK biomarker in a lupus patient population, confirmed at a second time point at least 3, 4, 5, or 6 months or 1-2 years apart by a mean average increase of from 0.6-0.7, or an increase in a range of from about 0.20 to about 0.95 (e.g., 0.25 to 0.90, 0.3 to 0.8, 0.4 to 0.7, or 0.5-0.6), of the SLEDAI score (e.g., a mean SLEDAI score);

c. Neutrophil gene signature: the measure of the neutrophil gene signature being in the top 15% of measures of the neutrophil gene signature in a lupus patient population, confirmed at a second time point at least 3, 4, 5, or 6 months or 1-2 years apart by a mean average increase of from 0.5-0.6, or an increase in a range of from about 0.15 to about 1.5 (e.g., 0.2 to 1.15, 0.3 to 1, 0.4 to 0.9, 0.5-0.8, or 0.6 to 0.7) of the SLEDAI score (e.g., a mean SLEDAI score);

d. Interferon gene signature: An increase in the measure of the IFN signature of about 1 standard deviation (SD) relative to reference value (e.g., a median value for a lupus patient population) is indicative of an increase in a range of from about 0.01 to about 0.30, 0.05 to 0.20, 0.08 to 0.15, 0.1 to 0.12, or about 0.11 of the renal SLEDAI score (e.g., mean renal SLEDAI score); and/or

e. An increase in the measure of the neutrophil signature of about 1 standard deviation (SD) relative to reference value (e.g., a median value for a lupus patient population) in the measure of the TWEAK biomarker (e.g., urinary TWEAK) is indicative of an increase in a range of from about 0.10 to about 0.35, 0.15 to 0.3, 0.20 to 0.26, or about 0.25 of the renal SLEDAI score (e.g., mean renal SLEDAI score).

“Acquire” or “acquiring” as the terms are used herein, refer to obtaining possession of a physical entity (e.g., a sample, a polypeptide, a nucleic acid, or a sequence), or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

Lupus is “treated,” “inhibited” or “reduced,” if at least one symptom of the disease is reduced, alleviated, terminated, slowed, or prevented. As used herein, lupus is also “treated,” “inhibited,” or “reduced,” if recurrence or relapse of the disease is reduced, slowed, delayed, or prevented. Exemplary clinical symptoms of lupus that can be used to aid in determining the disease status in a subject can include e.g., painful joints/arthralgia, fever of more than 100° F./38° C., arthritis/swollen joints, prolonged or extreme fatigue, skin rashes, anemia, kidney involvement, pain in the chest on deep breathing/pleurisy, butterfly-shaped rash across the cheeks and nose, sun or light sensitivity/photosensitivity, hair loss, blood clotting problems, Raynaud's phenomenon/fingers turning white and/or blue in the cold, seizures, mouth or nose ulcers, and any combination thereof. Clinical signs of lupus are routinely classified and standardized, e.g., using an SLEDA rating system.

As used herein, the “Systemic Lupus Erythematosus Disease Activity Index” or “SLEDAI” is intended to have its customary meaning in the medical practice. EDSS is a rating system that is frequently used for classifying and standardizing MS. The accepted scores range from 0 (normal) to 105 (death due to lupus). A SLEDAI score of between 1-5 is indicative of mild disease activity in the subject; a SLEDAI score of between 6-10 is indicative of moderate disease activity in the subject; a SLEDAI score of between 11-19 is indicative of high disease activity in the subject; a SLEDAI score of 20-105 is indicative of very high disease activity in the subject.

“Responsiveness,” to “respond” to treatment, and other forms of this verb, as used herein, refer to the reaction of a subject to treatment with an lupus therapy. As an example, a subject responds to an lupus therapy if at least one symptom of lupus (e.g., disease progression) in the subject is reduced or retarded by about 10%, 20%, 30%, 40%, 50%, 60%, 70%, 80%, 90% or more. In another example, a subject responds to a lupus therapy, if at least one symptom of lupus in the subject is reduced by about 5%, 10%, 20%, 30%, 40%, 50% or more as determined by any appropriate measure, e.g., one or more of: a value of disease progression, a change in symptoms, and/or a modified SLEDAI value. In another example, a subject responds to treatment with a lupus therapy, if the subject has an increased time to progression. Several methods can be used to determine if a patient responds to a treatment including the assessments described herein, as set forth above.

An “overexpression” or “significantly higher level of expression” of the gene products refers to an expression level or copy number in a test sample that is greater than the standard error of the assay employed to assess the level of expression. In embodiments, the overexpression can be at least two, at least three, at least four, at least five, or at least ten or more times the expression level of the gene in a control sample or the average expression level of gene products in several control samples.

The term “probe” refers to any molecule which is capable of selectively binding to a specifically intended target molecule, for example a marker of the invention. Probes can be either synthesized by one skilled in the art, or derived from appropriate biological preparations. For purposes of detection of the target molecule, probes can be specifically designed to be labeled, as described herein. Examples of molecules that can be utilized as probes include, but are not limited to, RNA, DNA, proteins, antibodies, and organic monomers.

A “progressor” refers to a subject who possesses a disease progression value reflecting at least one, two, three, four or all of the following criteria:

a. BAFF: An increase in the measure of the BAFF biomarker (e.g., a BAFF mRNA) of about 1 standard deviation (SD) relative to reference value (e.g., a median value for a lupus patient population), confirmed at a second time point at least 3, 4, 5, or 6 months or 1-2 years apart by a mean average increase of from 0.2-0.3, or an increase in a range of from about 0.05 to about 0.45 (e.g., 0.09 to 0.40, 0.15 to 0.35, 0.2 to 0.3, or 0.25-0.26), of the SLEDAI score;

b. TWEAK: the measure of the TWEAK biomarker being in the top 15% of measures of the TWEAK biomarker in a lupus patient population, confirmed at a second time point at least 3, 4, 5, or 6 months or 1-2 years apart by a mean average increase of from 0.6-0.7, or an increase in a range of from about 0.20 to about 0.95 (e.g., 0.25 to 0.90, 0.3 to 0.8, 0.4 to 0.7, or 0.5-0.6), of the SLEDAI score;

c. Neutrophil gene signature: the measure of the neutrophil gene signature being in the top 15% of measures of the neutrophil gene signature in a lupus patient population, confirmed at a second time point at least 3, 4, 5, or 6 months or 1-2 years apart by a mean average increase of from 0.5-0.6, or an increase in a range of from about 0.15 to about 1.5 (e.g., 0.2 to 1.15, 0.3 to 1, 0.4 to 0.9, 0.5-0.8, or 0.6 to 0.7) of the SLEDAI score;

d. Interferon gene signature: An increase in the measure of the IFN signature of about 1 standard deviation (SD) relative to reference value (e.g., a median value for a lupus patient population) is indicative of an increase in a range of from about 0.01 to about 0.30, 0.05 to 0.20, 0.08 to 0.15, 0.1 to 0.12, or about 0.11 of the renal SLEDAI score (e.g., mean renal SLEDAI score); and/or

e. An increase in the measure of the neutrophil signature of about 1 standard deviation (SD) relative to reference value (e.g., a median value for a lupus patient population) in the measure of the TWEAK biomarker (e.g., urinary TWEAK) is indicative of an increase in a range of from about 0.10 to about 0.35, 0.15 to 0.3, 0.20 to 0.26, or about 0.25 of the renal SLEDAI score (e.g., mean renal SLEDAI score).

Baseline values for the aforementioned tests (e.g., BAFF biomarker, TWEAK biomarker, and neutrophil gene signature) can be determined using the best baseline value or the average baseline value (e.g., a lupus patient population.

“Sample,” “tissue sample,” “patient sample,” “patient cell or tissue sample” or “specimen” each refers to a biological sample obtained from a tissue or bodily fluid of a subject or patient. The source of the tissue sample can be solid tissue as from a fresh, frozen and/or preserved organ, tissue sample, biopsy, or aspirate; blood or any blood constituents (e.g., serum, plasma); bodily fluids such as urine, cerebral spinal fluid, whole blood, plasma and serum. The sample can include a non-cellular fraction (e.g., urine, plasma, serum, or other non-cellular body fluid). In one embodiment, the sample is a urine sample. In other embodiments, the body fluid from which the sample is obtained from an individual comprises blood (e.g., whole blood). In certain embodiments, the blood can be further processed to obtain plasma or serum. In another embodiment, the sample contains a tissue, cells (e.g., peripheral blood mononuclear cells (PBMC)). In one embodiment, the sample is a urine sample. For example, the sample can be a fine needle biopsy sample, an archival sample (e.g., an archived sample with a known diagnosis and/or treatment history), a histological section (e.g., a frozen or formalin-fixed section, e.g., after long term storage), among others. The term sample includes any material obtained and/or derived from a biological sample, including a polypeptide, and nucleic acid (e.g., genomic DNA, cDNA, RNA) purified or processed from the sample. Purification and/or processing of the sample can involve one or more of extraction, concentration, antibody isolation, sorting, concentration, fixation, addition of reagents and the like. The sample can contain compounds that are not naturally intermixed with the tissue in nature such as preservatives, anticoagulants, buffers, fixatives, nutrients, antibiotics or the like.

The amount of a biomarker, e.g., expression of gene products (e.g., one or more the biomarkers described herein), in a subject is “significantly” higher or lower than the normal amount of a marker, if the amount of the marker is greater or less, respectively, than the normal level by an amount greater than the standard error of the assay employed to assess amount, or at least two, three, four, five, ten or more times that amount. Alternatively, the amount of the marker in the subject can be considered “significantly” higher or lower than the normal amount if the amount is at least about 1.5, two, at least about three, at least about four, or at least about five times, higher or lower, respectively, than the normal amount of the marker.

As used herein, “significant event” shall refer to an event in a patient's disease that is important as determined by one skilled in the art. Examples of significant events include, for example, without limitation, primary diagnosis, death, flares, or the progression of a patient's disease from any one of the above noted stages to another. A significant event can be any important event used determine disease status using e.g., SLEADAI or other symptom criteria, as described herein or determined by one skilled in the art.

As used herein, “time course” shall refer to the amount of time between an initial event and a subsequent event. For example, with respect to a patient's disease, time course can relate to a patient's disease and can be measured by gauging significant events in the course of the disease, wherein the first event can be diagnosis and the subsequent event can be a flare, for example.

A “transcribed polynucleotide” is a polynucleotide (e.g., an RNA, a cDNA, or an analog of one of an RNA or cDNA) which is complementary to or homologous with all or a portion of a mature RNA made by transcription of a marker of the invention and normal post-transcriptional processing (e.g., splicing), if any, of the transcript, and reverse transcription of the transcript.

An “underexpression” or “significantly lower level of expression” of products (e.g., the markers set forth herein) refers to an expression level in a test sample that is greater than the standard error of the assay employed to assess expression, for example, at least 1.5, twice, at least three, at least four, at least five, or at least ten or more times less than the expression level of the gene in a control sample, or the average expression level of gene products in several control samples.

Various aspects of the invention are described in further detail below. Additional definitions are set out throughout the specification.

### Lupus and Methods of Diagnosis

Lupus is an inflammatory disorder of autoimmune etiology, occurring predominantly in young women. Lupus can affect many of the body's organ systems, including kidneys, skin, joints, nervous system, serous membranes, blood cells and vessels. Although the specific cause of lupus is unknown, multiple factors are associated with the development of the disease, including genetic, racial, hormonal, and environmental factors.

The course of lupus is usually chronic, relapsing, and unpredictable. Untreated lupus can be fatal as it progresses from attack of skin and joints to internal organs, including lung, heart, and kidneys, thus making early and accurate diagnosis of and/or assessment of risk of developing lupus particularly critical. Lupus mainly appears as a series of flare-ups, with intervening periods of little or no disease manifestation. Kidney damage, measured by the amount of protein in the urine, is one of the most acute areas of damage associated with pathogenicity in lupus, and accounts for at least 50% of the mortality and morbidity of the disease.

Lupus is characterized by the production of unusual autoantibodies in the blood. Over 100 different self-molecules have been found to bind autoantibodies in different patients (Sherer et al. (2004) Semin. Arthritis. Rheum. 34:501-37), forming immune complexes which circulate the blood and eventually deposit in tissues. These immune complex depositions cause chronic inflammation and eventually tissue damage. The autoantibodies also have direct pathogenic effects contributing to hemolytic anemia and thrombocytopenia.

Exemplary symptoms associated with lupus which can be treated with the methods described herein or managed using symptom management therapies, include, but are not limited to, achy joints/arthralgia, fever of more than 100° F./38° C., arthritis/swollen joints, prolonged or extreme fatigue, skin rashes, anemia, kidney involvement, pain in the chest on deep breathing/pleurisy, butterfly-shaped rash across the cheeks and nose, sun or light sensitivity/photosensitivity, hair loss, blood clotting problems, Raynaud's phenomenon/fingers turning white and/or blue in the cold, seizures, mouth or nose ulcers, and any combination thereof.

Typically, a diagnosis of lupus can be made on the basis of eleven criteria defined by the American College of Rheumatology (ACR). These criteria include malar rash, discoid rash, photosensitivity, oral ulcers, arthritis, serositis, renal disorder, neurologic disorder, hematologic disorder (e.g., leucopenia, lymphopenia, hemolytic anemia or thrombocytopenia), immunologic disorder and anti-nuclear antibodies (ANA) (Tan et al. (1997) Arthritis Rheum 1997 40:1725). A subject can be clinically diagnosed with lupus if he meets at least four of the eleven criteria. Nevertheless, lupus is still possible even in case when less than four criteria are present.

While anti-nuclear antibodies and autoantibodies to dsDNA, phospholipids and Sm proteins are among the eleven criteria used for diagnosing lupus (Tan et al.), many patients diagnosed with lupus lack these autoantibodies, especially when they are in clinical remission.

Lupus patients are also evaluated with the SLEDAI scale, described in more detail below.

### Systemic Lupus Erythematosus Disease Activity Index (SELENA SLEDAI)

The clinical score known as SELENA-SLEDAI is an index of SLE disease activity as measured and evaluated within the last 10 days (Bombardier, C et al. (1992) Arthritis Rheum 35:630-640). Disease activity under the SELENA-SLEDAI scoring system can range from 0 to 105. The following categories of SLEDAI activity have been defined: no activity (SLEDAI=0); mild activity (SELENA-SLEDAI=1-5); moderate activity (SELENA-SLEDAI=6-10); high activity (SELENA-SLEDAI=11-19); very high activity (SELENA-SLEDAI=20 or higher). (Griffiths, et al., Assessment of Patients with Systemic Lupus Erythematosus and the use of Lupus Disease Activity Indices). In some embodiments the SELENA-SLEDAI is modified to exclude complement and dsDNA (modSLEDAI) as described in, e.g., Aggarwal, R, et al., (2011) Arthritis Care & Research 63(6):891-898.

In certain instances, moderate SLE is defined as patients with SLEDAI scores of between and including 6-10. Alternatively, moderate SLE may be defined as patients with SLEDAI scores of between and including 5-10, 5-11, 5-12, 6-11, or even 6-12.

In certain instances, severe SLE is defined as patients with SELENA-SLEDAI scores of between and including 11-19. Alternatively, severe SLE may be defined as patients with SELENA-SLEDAI scores of between and including 12-20, or even 13-20.

In certain instances, very severe SLE is defined as patients with SELENA-SLEDAI scores of greater than 20.

Table 1 below shows the criteria for determining the SELENA-SLEDAI score. Total score is sum of weights next to descriptors marked present.

TABLE 1
The SELENA-SLEDAI scale as used to identify lupus flares. Total score is sum
of weights next to descriptors marked present.

| Weight | Descriptor | Definition |
|---|---|---|
| 8 | Seizure | Recent onset. Exclude metabolic, infectious or drug cause. |
| 8 | Psychosis | Altered ability to function in normal activity due to severe |
| | | disturbance in the perception of reality. Include hallucinations, |
| | | incoherence, marked loose associations, impoverished thought |
| | | content, marked illogical thinking, bizarre, disorganized, or |
| | | catatonic behavior. Excluded uremia and drug causes. |
| 8 | Organic Brain | Altered mental function with impaired orientation, memory or other |
| | Syndrome | intelligent function, with rapid onset fluctuating clinical features. |
| | | Include clouding of consciousness with reduced capacity to focus, |
| | | and inability to sustain attention to environment, plus at least two of |
| | | the following: perceptual disturbance, incoherent speech, insomnia |
| | | or daytime drowsiness, or increased or decreased psychomotor |
| | | activity. Exclude metabolic, infectious or drug causes. |
| 8 | Visual | Retinal changes of SLE. Include cytoid bodies, retinal hemorrhages, |
| | Disturbance | serious exodate or hemorrhages in the choroids, or optic neuritis. |
| | | Exclude hypertension, infection, or drug causes. |
| 8 | Cranial Nerve | New onset of sensory or motor neuropathy involving cranial nerves. |
| | Disorder | |
| 8 | Lupus Headache | Severe persistent headache: may be migrainous, but must be |
| | | nonresponsive to narcotic analgesia. |
| 8 | CVA | New onset of cerebrovascular accident(s). Exclude arterio. |
| 8 | Vasculitis | Ulceration, gangrene, tender finger nodules, periungual, infarction, |
| | | splinter hemorrhages, or biopsy or angiogram proof of vasculitis. |
| 4 | Arthritis | More than 2 joints with pain and signs of inflammation (i.e. |
| | | tenderness, swelling, or effusion). |
| 4 | Myositis | Proximal muscle aching/weakness, associated with elevated creatine |
| | | phosphokinase/adolase or electromyogram changes or a biopsy |
| | | showing myositis. |
| 4 | Urinary Casts | Heme-granular or red blood cell casts. |
| 4 | Hematuria | >5 red blood cells/high power field. Exclude stone, infection or |
| | | other cause. |
| 4 | Proteinuria | >0.5 gm/24 hours. New onset or recent increase of more than 0.5 |
| | | gm/24 hours. |
| 4 | Pyuria | >5 white blood cells/high power field. Exclude infection. |
| 2 | New Rash | New onset or recurrence of inflammatory type rash. |
| 2 | Alopecia | New onset or recurrence of abnormal, patchy or diffuse loss of hair. |
| 2 | Mucosal Ulcers | New onset or recurrence of oral or nasal ulcerations. |
| 2 | Pleurisy | Pleuritic chest pain with pleural rub or effusion, or pleural |
| | | thickening. |
| 2 | Pericarditis | Pericardial pain with at least 1 of the following: rub, effusion, or |
| | | electrocardiogram confirmation. |
| 2 | Low Complement | Decrease in CH50, C3, or C4 below the lower limit of normal for |
| | | testing laboratory. |
| 2 | Increased DNA | >25% binding by Farr assay or above normal range for testing |
| | binding | laboratory. |
| 1 | Fever | >38° C. Exclude infectious cause. |
| 1 | Thrombo- | <100,000 platelets/mm³. |
| | cytopenia | |
| 1 | Leukopenia | <3,000 White blood cell/mm³. Exclude drug causes. |

Thus, while SELENA-SLEDAI is sufficient to determine current disease status and whether a flare has recently occurred, is insufficient to investigate future lupus disease status or therapeutic efficacy of treatments. Clinical measures that supplement the sensitivity of this endpoint are required in order to adequately characterize progressive forms of lupus and evaluate progressor status, as well as treatment effects on disease progression in progressive forms of lupus.

### Use of Biomarkers to Evaluate Disease Progression

As highlighted above, in order to evaluate a beneficial effect of a treatment in preventing the progression of disability in lupus, the endpoints selected must have adequate sensitivity to detect worsening disease in the studied population. In one embodiment, one, two or all three of a BAFF biomarker, a TWEAK biomarker, and a neutrophil gene signature can be used to acquire a disease progression value. The disease progression value can be used for, e.g., in characterizing progressive forms of lupus and evaluating the effectiveness of therapies in treating lupus.

In some embodiments, methods of the present invention can be used to identify a subject as disease progressing, wherein if a sample in a subject has a significant increase in the amount of one, two, three or all four of a BAFF biomarker, a TWEAK biomarker, a neutrophil gene signature, and an interferon signature relative to a standard, e.g., a median value for a lupus patient population, then the disease is more likely to progress. BAFF, TWEAK, neutrophil gene and interferon signatures are each described in further detail below.

### Disease Progression Value

The disease progression value disclosed herein can be used as a means to identify lupus patients as disease progressing. This can be used in lupus patient evaluation and as a primary endpoint for use in clinical trials, as well as in the evaluation of the effects of a treatment in preventing the progression of disease in the clinic. The disease progression value can include individual component levels of BAFF, TWEAK, and neutrophils.

In certain embodiments, patients who are disease progressing possess a disease progression value reflecting the following criteria:
a. an increase in the measure of a BAFF biomarker relative to a reference; of about 1 standard deviation (SD) relative to reference value (e.g., a median value for a lupus patient population)
b. an increase in the measure of a BAFF biomarker relative to a reference; and
c. an increase in the measure of a neutrophil gene signature relative to a reference.

In some embodiments, an increase in the measure of a BAFF biomarker comprises an increase of 0.1, 0.2, 0.3, 0.4, 0.5, 0.6, 0.7, 0.8, 0.9, 1.0, 2, or more standard deviations (SD) relative to reference value. In one embodiment, the increase in the measure of the BAFF biomarker is about 1 SD.

In some embodiments a reference value comprises a baseline or prior value for the subject, the subject at a different time interval, an average or median value for a lupus patient population, a healthy control, or a healthy subject population. In some embodiments, a reference value comprises a median value lupus patient population.

In some embodiments, an increase in the measure of a TWEAK biomarker comprises being in the top 1%, 5%, 10%, 15%, 20%, 25%, or 30% of measures of the TWEAK biomarker in a reference population. In some embodiments, the measure of the TWEAK biomarker being in the top 15% of measures of the TWEAK biomarker in a reference population.

In some embodiments, an increase in the measure of a neutrophil gene signature comprises being in the top 1%, 5%, 10%, 15%, 20%, 25%, or 30% of measures of the neutrophil gene signature in a reference population. In some embodiments, the measure of the neutrophil gene signature being in the top 15% of measures of the neutrophil gene signature in a reference population.

In some embodiments, an increase in the measure of the IFN signature of about 1 standard deviation (SD) relative to reference value (e.g., a median value for a lupus patient population) is indicative of an increase in a range of from about 0.01 to about 0.30, 0.05 to 0.20, 0.08 to 0.15, 0.1 to 0.12, or about 0.11 of the renal SLEDAI score (e.g., mean renal SLEDAI score).

In other embodiments, an increase in the measure of the neutrophil signature of about 1 standard deviation (SD) relative to reference value (e.g., a median value for a lupus patient population) in the measure of the TWEAK biomarker (e.g., urinary TWEAK) is indicative of an increase in a range of from about 0.10 to about 0.35, 0.15 to 0.3, 0.20 to 0.26, or about 0.25 of the renal SLEDAI score (e.g., mean renal SLEDAI score).

In some embodiments, a reference population comprises a healthy population. In some embodiments, a reference population comprises a population of individuals free of lupus symptoms. In some embodiments, a reference population comprises a lupus patient population.

One exemplary reference population comprising a lupus patient population is the SPARE study. The SPARE study was a prospective, longitudinal, observational study conducted at one study center (Hopkins Lupus Center) in the United States. Adult patients were eligible if they were aged 18 to 75 years; met the American College of Rheumatology (ACR) Revised Criteria for Classification of Systemic Lupus Erythematosus; were enrolled in the Hopkins Lupus Cohort at Johns Hopkins Hospital, Baltimore, Md., USA; and provided written informed consent (Tan et al.). Patients who were treated according to standard clinical practice underwent clinical laboratory and biomarker assessments for 2 years: samples were taken at baseline; at months 3, 6, 9, 12, 15, 18, 21, and 24; and at unscheduled visits associated with flares. The analysis presented herein are based on the first 12 months of data from this cohort.

In the setting of a clinical trial or other patient evaluation, a possible progression is confirmed when the defined minimum change is reached. In some embodiments, disease progression value is confirmed at least 3, 4, 5 or 6 months or 1 or 2 years after progression value assessment. In some embodiments, progression is confirmed 1 year after disease progression value assessment.

If the value at the subsequent visit does not reach the defined minimum change the progression may not be confirmed. In some embodiments, the defined minimum change is 0.1, 0.5 or 1 on the SLEDAI. In some embodiments, the defined minimum change is 0.1 on the SLEDAI. To identify a possible progression, all study visits (except unscheduled visits for relapse assessments) may be used.

### Subjects

For any of the methods and kits disclosed herein, the subject treated, or the subject from which the value is obtained, is a subject having, or at risk of having, lupus at any stage of treatment. In certain embodiments, the subject is a patient having lupus. In another embodiment, the subject is asymptomatic. In other embodiments, the subject has one or more lupus-like symptoms. In other embodiments, the subject is a subject known to have, or suspected of having, or at increased risk of developing, lupus. A subject can also include a subject not previously known or suspected to have lupus, or in need of treatment for lupus. A subject can also be a subject known to have or believed to be at risk of developing lupus. Subjects described herein as being at risk of developing lupus are identified by family history, genetic analysis, environmental exposure and/or the onset of early symptoms associated with the disease or disorder described herein.

### Biomarker Assessments

### BAFF

BAFF (Also Known as TNFSF13B and BLyS):

The nucleotide and protein sequences of human BAFF are disclosed e.g., in Schneider, P et al. (1999) J Exp Med 189:1747-1756; Moore, P A et al. (1999) Science 285:260-263; and Tribouley, C et al. (1999) Biol Chem 380(12):1443-1447. BAFF is a cytokine involved in the stimulation of B- and T-cell function for the regulation of humoral immunity, and promotes the survival of mature B-cells. BAFF is highly expressed in peripheral blood leukocytes and in monocytes and macrophages. BAFF is also expressed in the spleen, lymph node, bone marrow, T-cells, and dendritic cells. Antibodies for BAFF can be obtained through a variety of commercial sources including, e.g., Abcam®, Acris Antibodies™, GeneTex™, LifeSpan Biosciences™, Santa Cruz Biotechnology® and Sigma-Aldrich®.

B cells play a central role in the pathogenesis of SLE. Cancro et al. (2009). J Clin Invest 119:1066-1073. Serum BAFF levels are elevated in ˜50% of patients with SLE and appear to correlate with anti-double-stranded DNA (anti-dsDNA) titers and disease activity according to the Systemic Lupus Erythematosus Disease Activity Index (SLEDAI) (Petri et al. (2008) Arthritis Rheum 58:2453-2459; Stohl et al. (2003) Arthritis Rheum 48:3475-3486). As a result, one therapeutic strategy for targeting B cells in patients with SLE is through the inhibition of BAFF. An anti-BAFF antibody has shown significant efficacy in two Phase III clinical trials for moderately active SLE (Navarra et al. (2011) Lancet 377:721-731). Other inhibitors of BAFF are being developed or are at early stages of clinical testing (Cancro et al. (2009) J Clin Invest 119:1066-1073).

The association between BAFF gene expression and clinical observations in SLE patients was evaluated in the SPARE study. To evaluate clinical associations of BAFF gene expression, peripheral blood RNA samples were profiled using Affymetrix chips, and the BAFF gene expression levels at baseline were divided into low, median, and high groups based on BAFF score distribution.

A BAFF “score” was calculated for each patient, based on the expression levels of the BAFF gene. The top 25% of the patients on the score distribution plot, with scores >11.4, were considered to have a high BAFF gene expression; the bottom 25% with scores <10.7 had a low BAFF expression; patients with scores between 10.7 and 11.4 had a medium BAFF expression.

Clinical associations, based on the cumulative history and the same-day visit disease activity, were determined using the chi-square test with Statistical Analysis Software version 9.2 (SAS Institute, Cary, N.C., USA). P-values were adjusted for ethnicity. A P-value of ≦0.05 was considered statistically significant.

Statistical analysis was done to determine whether there was a correlation between BAFF and the 11 SLE classification criteria developed by the ACR for classifying SLE using cumulative medical history and SELENA-SLEDAI scores.

A second analysis of the association between BAFF gene expression and clinical observations in SLE patients was completed in the SPARE study. To evaluate clinical associations of BAFF gene transcript expression, peripheral whole blood RNA samples were collected at baseline in PAXgene Blood RNA tubes and profiled using Affymetrix chips. The BAFF transcript was measured by two probes on the microarray, and were highly concordant. To determine BAFF transcript expression, a single probe was used (BAFF Affymetrix probe 223501_PM_at). Based on the distribution, intensity by Affymetrix profiling >11.4 was considered high BAFF mRNA expression (n=84), an intensity of 10.7 to 11.4 was considered medium BAFF mRNA expression (n=110), and an intensity <10.7 was considered low BAFF mRNA expression (n=98). BAFF transcript expression was verified by qPCR.

Clinical associations with BAFF over the subsequent year were determined using a generalized estimating equation to account for the repeated observations among the same patients. Disease activity was measured by PGA and Safety of Estrogen in Lupus Erythematosus-National Assessment (SELENA) SLEDAI at each visit. The PGA threshold of >1 reflected any disease activity of greater than mild severity. The SELENA SLEDAI threshold of ≧2 reflected disease activity of mild severity or greater P-values were adjusted for ethnicity where indicated.

Further embodiments for measuring BAFF biomarkers are described below.

### TWEAK

TNF-like weak inducer of apoptosis (TWEAK) is a member of the TNF family of ligands. TNF family members play a role in the regulation of the immune system, controlling cell survival and differentiation, as well as acute host defense systems, such as inflammation. TWEAK was isolated in a screen for RNA that hybridized to an erythropoietin probe (Chicheportiche et al. (1997) J. Biol. Chem. 272:32401-32410). The mouse and human peptides have an unusually high degree of conservation, including 93% amino acid identity in the receptor binding domain. TWEAK, shown to be efficiently secreted from cells, is abundantly expressed in many tissues, including heart, brain, placenta, lung, liver, skeletal muscle, kidney, pancreas, spleen, lymph nodes, thymus, appendix, and peripheral blood lymphocytes.

TWEAK has been implicated in many biological processes. For instance, HT29 cells treated with IFN-γ and TWEAK were shown to undergo apoptosis; although TWEAK's ability to induce apoptosis is weak and only a small number of cell types are susceptible (Chicheportiche et al.). In contrast, TWEAK has also been shown to induce angiogenesis and proliferation of endothelial cells in a VEGF-independent pathway (Lynch et al. (1999) J. Biol. Chem. 274:8455-8459). Astrocytes are specifically bound and stimulated by TWEAK. TWEAK can infiltrate an inflamed brain to influence astrocyte behavior. Astrocytes exposed to TWEAK secrete high levels of IL-6 and IL-8, as well as upregulate ICAM-1 expression (Saas et al. (2000) GLIA 32:102-107).

TWEAK has also been implicated in immune system regulation. Upon stimulation with IFN-γ, monocytes rapidly express TWEAK, and anti-TWEAK antibodies partially inhibited their cytotoxic activity against human squamous carcinoma cells. A combination of anti-TWEAK and anti-TRAIL antibodies almost completely inhibited cytotoxicity (Nakayama et al. (2000) J. Exp. Med. 192:1373-1379. In contrast, TWEAK mRNA rapidly disappeared in mice treated with lipopolysaccharide (LPS), an inducer of the immuno-inflammatory responses. Furthermore, TWEAK mRNA was also reduced in autoimmune hemolytic anemia and systemic lupus erythematosus in mouse models. These data suggest that the down-regulation of TWEAK expression is an important event in acute and chronic inflammation (Chicheportiche et al. (2000) Biochem. Biophys. Res. Comm. 279:162-165).

The TWEAK/Fn14 pathway mediates key pathologic processes involved in renal disease in SLE. Previous studies have demonstrated that lupus patients with active renal disease have higher levels of urinary TWEAK (uTWEAK) compared to those without active renal disease. Furthermore, uTWEAK levels correlated significantly with renal disease activity on the same day as assessed by the renal SLE Disease Activity Index (SLEDAI) scores.

The association between urinary TWEAK levels and clinical observations in SLE patients was evaluated in the SPARE study. At baseline, uTWEAK levels were measured by ELISA and normalised with urinary creatinine. Patients within uTWEAK quartiles were compared with respect to the frequency of disease activity measured at clinic visits in the following year. Estimates of differences and p-values were based on generalized estimating equations (GEE) models to account for repeated visits from the same patient. Cox proportional hazards models were fit to time to first renal flare within the first year, adjusting for either log 2-transformed or quartile of uTWEAK along with sex, race, complement (C3, C4), and dsDNA; p-values for uTWEAK were based upon the effect estimates. Renal flares were defined as increase in SLEDAI renal descriptors or doubling of urine protein/cr.

Further embodiments for measuring TWEAK biomarkers are described below.

### Neutrophils

Neutrophils and neutrophil cell death (“NETosis”) are now understood to play a role in the pathogenesis of SLE. Neutrophils immobilize and kill invading microbes by releasing extracellular traps (NETs), which are made of DNA, histones, and neutrophil proteins. This unique type of neutrophil cell death is described as NETosis (Brinkmann et al. (2004) Science 303:1532-1535). Recently, a putative link between NETosis and autoimmunity has been proposed, due to the potential role of netting neutrophils in externalizing autoantigens, thereby making these molecules more exposed to the immune systems (Hakkim et al. (2010) Proc Natl Acad Sci USA 107:9813-9818). Microarray analysis has identified neutrophil gene signatures that correlated with the presence of low-density granulocytes (LDGs) in peripheral blood mononuclear cells of SLE patients (see e.g., Bennett et al. (2003) J Exp Med 197:711-723; Chaussabel et al. (2008) Immunity 29:150-164; Villanueva et al. (2011) J Immunol 187:538-552). A positive association was described between the neutrophil gene signature and disease activity in a small cohort of pediatric SLE patients (n=22) according to the Systemic Lupus Erythematosus Disease Activity Index (SLEDAI) (Chaussabel et al.). Tissue NETosis appeared to be associated with increased anti-double-stranded DNA (anti-dsDNA) in sera (Villanueva et al.). Additional reports of the high LDG-associated neutrophil signature (which includes BPI, CEACAM6, CEACAM8, DEFA4, LCN2, LTF, MMP8, and OLFM4) and its association with SLE disease activity, are provided in Petri et al. “Association between Neutrophil Gene Signature and Disease Characteristics in SLE Patients” presented at the 76^{th} Annual Scientific Meeting of the American College of Rheumatology (ACR November 2012); Petri et al. “A Neutrophil Signature Comprised of LDG-enriched genes associated with Organ-Specific Disease Activity in SLE” presented at the 77^{th} Annual Scientific Meeting of the American College of Rheumatology (ACR October 2013); ACR 2013 Slide presentation by Petri et al. entitled “Association of a Neutrophil Gene Signature Comprised of Low Density Granulocyte-enriched Genes with Both Future SLE Disease Activity and Poor Longterm Outcomes;” Associated abstracts: by Petri et al. Arthritis and Rheumatism Vol 65 October 2013, Abstract Supplement (see attached email) and ACR/ARHP 13 Annual meeting, abstract entitled “Association of a Neutrophil Gene Signature Comprised of Low Density Granulocyte-enriched Genes with Both Future SLE Disease Activity and Poor Longterm Outcomes” Abstract #882 (Oct. 27, 2013). The contents of all of aforesaid publications are incorporated by reference.

The association between neutrophil gene signature and disease characteristics in adult SLE patients was evaluated in the SPARE study. Neutrophil gene signature in this analysis was identified based on previous studies. The genes reported in literature to be upregulated in the LDG subset relative to normal-density neutrophils were selected (Bennett et al. supra; Chaussabel et al. supra; Villanueva, E et al., supra).

From this initial list, gene transcripts that correlate with each other with a coefficient >0.7 in the SPARE data were chosen to define the neutrophil gene signature in this analysis. At baseline, gene expression levels were assessed in peripheral blood RNA samples collected using PAXgene Blood RNA tubes and profiled using microarray (Affymetrix) and select transcripts validated with qPCR. Neutrophil signature genes chosen were BPI, CEACAM6, CEACAM8, DEFA4, LCN2, LTF, MMP8, and OLFM4. CEACAM6 can be detected using 2 Affymetrix probes.

The LDG signature “score” was calculated as the geometric mean of the expression levels (chip signal intensity) of the 8 genes in the signature. Patients with a score >6 were considered to have a high neutrophil gene signature, those with a score <5 had a low neutrophil gene signature, and patients with a score between 5 and 6 were considered to be medium.

Further embodiments for evaluating neutrophil gene signatures are described below.

### Interferon-Gene Signatures

In other embodiments, the value of disease progression comprises a measure of an IFN signature. In one embodiment, the interferon signature provides an indication of renal disease activity. The IFN signature (e.g., a type I Interferon signature or type II Interferon signature) can be used according to signatures known in the art, e.g., as described in Thurlings, et al. (2010) Arthritis and Rheumatology Vol. 62(12):3607-3614; Hall, J. C. et al. (2012) PNAS Vol. 109(43):17609-17614).

Type I interferons mediate the early immune response. type I interferons include IFN-α (alpha), IFN-β (beta), IFN-K (kappa), IFN-δ (delta), IFN-ε (epsilon), IFN-τ (tau), IFN-ω (omega), and IFN-ζ (zeta, also known as limitin).

Generally, activated Toll Like Receptors (TLRs) trigger type I interferon secretion from leukocytes, including plasmacytoid dendritic cells. In SLE, immune complex (IC) formation has been shown to induce IFN-alpha production by plasmacytoid dendritic cells. After secretion, type I interferon binds heterotrimeric type I interferon receptor on target cells, and tranduces signals mainly via JAK/STAT signaling pathways to initiate transcription of type I interferon stimulated genes, as described in, e.g., Ohl, et al., (2011) Journal of Biomedicine and Biotechnology 14 pages.

Without wishing to be held to any particular theory, type I interferons may contribute to SLE pathogenesis in several ways. IFN-alpha production induces feedback loops which promote production of type I interferons. IFN-alpha also promotes dendritic cell maturation, which reduces immune tolerance and increases levels of self-reactive T-cells and promotes survival of autoreactive B cells. Finally, IFN-alpha increases cytotoxicity of CD8 T cells and directly increases number of autoreactive CD4 T cells, as described in, e.g., Ohl, et al., (2011) Journal of Biomedicine and Biotechnology 14 pages.

In one embodiment, the interferon signature in an interferon-alpha gene signature. As described in the Examples below, the interferon-alpha gene signature can be calculated based on the geometric mean of the expression levels of 8 interferon alpha-induced genes, as defined by Hall, J. C. et al. (2012) PNAS Vol. 109(43):17609-17614). IFN alpha-induced genes that can be included in the calculation of the IFN-alpha gene signature include, e.g., IFIT1; MX2; OAS1; OAS2; OASL; HERCS; HES4; and/or RSAD2.

Type II interferons include IFN-γ (gamma). type II interferons potentiate the action of type I interferons. IFN-gamma also activates macrophages at the site of inflammation, contributes to cytotoxic T-cell activity, and promotes the Th1-response, inducing organ specific autoimmunity. In a mouse model of SLE, treatment with IFN-gamma accelerated development of the disease while administration of an anti IFN-gamma antibody resulted in remission as described in, e.g., Ohl, et al., (2011) Journal of Biomedicine and Biotechnology 14 pages.

The majority of genes that are highly upregulated by IFN-alpha are also highly induced by IFN-gamma. A substantial group of genes induced by IFN-gamma are not highly induced by IFN-alpha. In some embodiments, the interferon signature in an interferon-gamma gene signature. As described in the Examples below, the interferon-gamma gene signature can be calculated based on the geometric mean of the expression levels of a set of interferon gamma-induced genes, as defined by Hall, J. C. et al. (2012) PNAS Vol. 109(43):17609-17614). IFN gamma-induced genes that can be included in the calculation of the IFN-gamma gene signature include, e.g., AIM2; CD74; CXCL10; CXCL9; GBP2; GBP4; GBP5; HLA-DMA; HLA-DRA; HLA-DRB4; HLA-DRB6; IL18BP; INDO/IDO1; IRF1; LOC400759; PSMB10; RARRES3; SERPINB2; TNFRSF14; and/or WARS.

### Plasma Cells

SLE patients have multiple B cell abnormalities and produce a variety of autoantibodies directed against nuclear, cytoplasmic and cell surface autoantigens (Odendahl et al. (2000) J Immunol 165: 5970-5979; Jacobi et al. (2010) Ann Rheum Dis 69: 305-308). B cell abnormalities include an increase in the number of circulating plasma cells (PC). It is known that frequency of circulating PC correlates with SLEDAI as shown, e.g., in Jacobi et al. (2003) Arthritis Rheum 48: 1332-1342. Mature PCs exhibit a pattern of gene expression that includes up-regulation of genes limiting apoptosis linked to the unfolded protein response and ER stress, genes involved in cell cycle arrest, and genes involved in PC horning to tissue niches such as CXCR4 and SDC1, and downregulation of genes encoding specific B cell surface receptors, such as the BCR complex, B cell co-receptor (CD19, CD81, CD21), CD20 (MS4A1) and HLA Class II (Shapiro-Shelef et al. (2005) J Exp Med 202: 1471-1476; Shaffer et al, (2004) immunity 21: 81-93; Radbruch et al. (2006) Nat Rev Immunol 6: 741-750).

### Analysis of Lupus Biomarkers

Analysis of levels of expression and/or activity of gene products correlated with the disease progression of lupus has led to the identification of one, two, or all three of BAFF, TWEAK, and neutrophil and interferon gene signatures. For example, the present invention provides methods for evaluation of expression level of one, two, or all three BAFF and genes that comprise the neutrophil and/or interferon gene signature, and TWEAK protein expression or activity.

In some embodiments, methods of the present invention can be used to determine the responsiveness of a subject to treatment with a lupus therapy, wherein if a sample in a subject has a significant increase in the amount, e.g., expression, and/or activity of a marker disclosed herein relative to a reference, e.g., a median value for a lupus patient population, then the disease is more likely to respond to treatment.

The methods provided herein are particularly useful for identifying subjects that are likely to respond to lupus therapy prior to initiation of such treatment (e.g., pre-therapy) or early in the therapeutic regimen. In some embodiments, expression or activity of biomarkers is measured in a subject at least 2 weeks, at least 1 month, at least 3 months, at least 6 months, or at least 1 year after initiation of therapy. In some embodiments, it is preferred that expression or activity of biomarkers is measured less than 6 months after initiation of therapy to permit the skilled practitioner to switch the subject to a different therapeutic strategy. Thus, in some embodiments it is preferred that expression or activity of biomarkers is measured within 1-6 months, 1-5 months, 1-4 months, 1-3 months, 1-2 months, 2-6 months, 3-6 months, 4-6 months, 5-6 months, 2-3 months, 3-4 months, or 4-5 months of initiation of therapy. In some embodiments, the expression or activity of biomarkers is determined 3-6 months after initiation of therapy (e.g., 3 months, 3.5 months, 4 months, 4.5 months, 5 months, 5.5 months, 6 months).

The methods described herein can also be used to monitor a positive response of a subject to treatment. Such methods are useful for early detection of tolerance to therapy or to predict whether disease in a subject will progress. In such embodiments, the expression or activity of biomarkers is determined e.g., at least every week, at least every 2 weeks, at least every month, at least every 2 months, at least every 3 months, at least every 4 months, at least every 5 months, at least every 6 months, at least every 7 months, at least every 8 months, at least every 9 months, at least every 10 months, at least every 11 months, at least every year, at least every 18 months, at least every 2 years, at least every 3 years, at least every 5 years or more. It is also contemplated that expression or activity of the biomarkers is at irregular intervals e.g., biomarkers can be detected in an individual at 3 months of treatment, at 6 months of treatment, and at 7 months of treatment. Thus, in some embodiments, the expression or activity of the biomarkers is determined when deemed necessary by the skilled physician monitoring treatment of the subject.

The methods described herein can be used in any subject having lupus including systemic lupus erythematosus (SLE) (CLE includes, e.g., lupus nephritis), cutaneous lupus erythematosus (CLE) (CLE includes, e.g., acute cutaneous lupus erythematosus (ACLE), subacute cutaneous lupus erythematosus (SCLE), intermittent cutaneous lupus erythematosus, and chronic cutaneous lupus), drug-induced lupus, and neonatal lupus. About 70% of all cases of lupus are SLE. CLE can have symptoms that are limited to the skin or can be seen in those with SLE.

A subject that is identified as a disease progressor using the methods described herein can be treated with any treatment known in the art presently or to be developed. In some embodiments, a treatment comprises one or more of lupus therapy comprises one or more of: a nonsteroidal anti-inflammatory drug (NSAID); an antimalarial, including, for example, hydroxychloroquine; a corticosteroid; an immunosuppressant, including, for example, azathioprine, mycophenolate mofetil, or methotrexate; an intravenous immunoglobulin; an anti-TWEAK antibody; an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; or an anti-BAFF antibody.

In some embodiments, the amount of the biomarker determined in a sample from a subject is quantified as an absolute measurement (e.g., ng/mL). Absolute measurements can easily be compared to a reference value or cut-off value. For example, a cut-off value can be determined that represents a disease progressing status; any absolute values falling either above (i.e., for biomarkers that increase expression with progression of lupus) or falling below (i.e., for biomarkers with decreased expression with progression of lupus) the cut-off value are likely to be disease progressing.

Alternatively, the relative amount of a biomarker is determined. In one embodiment, the relative amount is determined by comparing the expression and/or activity of one or more biomarkers in a subject with lupus to the expression of the biomarkers in a reference parameter. In some embodiments, a reference parameter is obtained from one or more of: a baseline or prior value for the subject, the subject at a different time interval, an average or median value for a lupus patient population, a healthy control, or a healthy subject population.

The present invention also pertains to the field of predictive medicine in which diagnostic assays, pharmacogenomics, and monitoring clinical trials are used for predictive purposes to thereby treat an individual prophylactically. Accordingly, one aspect of the present invention relates to assays for determining the amount, structure, and/or activity of polypeptides or nucleic acids corresponding to one or more markers of the invention, in order to determine whether an individual having lupus or at risk of developing lupus will be more likely to respond to therapy.

Accordingly, in one aspect, the invention is drawn to a method for determining whether a subject with lupus is likely to respond to treatment. In another aspect, the invention is drawn to a method for predicting a time course of disease. In still another aspect, the method is drawn to a method for predicting a probability of a significant event in the time course of the disease (e.g., flare). In certain embodiments, the method comprises detecting a combination of biomarkers associated with responsiveness to treatment as described herein and determining whether the subject is likely to respond to treatment.

In some embodiments, the methods involve evaluation of a biological sample e.g., a sample from a subject, e.g., a patient who has been diagnosed with or is suspected of having lupus (e.g., presents with symptoms of lupus) to detect changes in expression and/or activity of one, two or all three of BAFF, TWEAK, and/or neutrophil gene signature biomarkers.

The results of the screening method and the interpretation thereof are predictive of the patient's disease progression. According to the present invention, alterations in expression or activity of one, two or all three of BAFF, TWEAK, and/or neutrophil gene signature is indicative of lupus progression relative to an average or median value for a lupus patient population.

In yet another embodiment, the one or more alterations, e.g., alterations in biomarker expression are assessed at pre-determined intervals, e.g., a first point in time and at least at a subsequent point in time. In one embodiment, a time course is measured by determining the time between significant events in the course of a patient's disease, wherein the measurement is predictive of whether a patient has a long time course. In another embodiment, the significant event is the progression from diagnosis to death. In another embodiment, the significant event is the progression from diagnosis to worsening disease. In another embodiment, the significant event is the progression from diagnosis to flare. In certain embodiments, the time course is measured with respect to one or more overall survival rate, time to progression and/or using the SLEDAI or other assessment criteria.

### Methods for Detection of Gene Expression

Biomarker expression level can also be assayed. Expression of a marker of the invention can be assessed by any of a wide variety of well known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In certain embodiments, activity of a particular gene is characterized by a measure of gene transcript (e.g., mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

Methods of detecting and/or quantifying the gene transcript (mRNA or cDNA made therefrom) using nucleic acid hybridization techniques are known to those of skill in the art (see e.g., Sambrook et al. supra). For example, one method for evaluating the presence, absence, or quantity of cDNA involves a Southern transfer as described above. Briefly, the mRNA is isolated (e.g., using an acid guanidinium-phenol-chloroform extraction method, Sambrook et al. supra.) and reverse transcribed to produce cDNA. The cDNA is then optionally digested and run on a gel in buffer and transferred to membranes. Hybridization is then carried out using the nucleic acid probes specific for the target cDNA.

A general principle of such diagnostic and prognostic assays involves preparing a sample or reaction mixture that can contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

In order to conduct assays with the above-mentioned approaches, the non-immobilized component is added to the solid phase upon which the second component is anchored. After the reaction is complete, uncomplexed components can be removed (e.g., by washing) under conditions such that any complexes formed will remain immobilized upon the solid phase. The detection of marker/probe complexes anchored to the solid phase can be accomplished in a number of methods outlined herein.

In another embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule can simply utilize the natural fluorescent energy of tryptophan residues. Labels are chosen that emit different wavelengths of light, such that the ‘acceptor’ molecule label can be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes can be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques can also be utilized to separate complexed molecules from uncomplexed ones. For example, gel filtration chromatography separates molecules based on size, and through the utilization of an appropriate gel filtration resin in a column format, for example, the relatively larger complex can be separated from the relatively smaller uncomplexed components. Similarly, the relatively different charge properties of the marker/probe complex as compared to the uncomplexed components can be exploited to differentiate the complex from uncomplexed components, for example, through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis can also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typical. Appropriate conditions to the particular assay and components thereof will be well known to one skilled in the art.

In a particular embodiment, the level of mRNA corresponding to the marker can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated

RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated nucleic acid can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

The probes can be full length or less than the full length of the nucleic acid sequence encoding the protein. Shorter probes are empirically tested for specificity. Exemplary nucleic acid probes are 20 bases or longer in length (See, e.g., Sambrook et al. for methods of selecting nucleic acid probe sequences for use in nucleic acid hybridization). Visualization of the hybridized portions allows the qualitative determination of the presence or absence of cDNA.

An alternative method for determining the level of a transcript corresponding to a marker of the present invention in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. Fluorogenic rtPCR can also be used in the methods of the invention. In fluorogenic rtPCR, quantitation is based on amount of fluorescence signals, e.g., TaqMan and sybr green. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations can be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a healthy subject, or between samples from different sources.

Alternatively, the expression level can be provided as a relative expression level. To determine a relative expression level of a marker, the level of expression of the marker is determined for 10 or more samples of normal versus lupus isolates, or even 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

In certain embodiments, the samples used in the baseline determination will be from samples derived from a subject having lupus versus samples from a healthy subject of the same tissue type. The choice of the cell source is dependent on the use of the relative expression level. Using expression found in normal tissues as a mean expression score aids in validating whether the marker assayed is specific to the tissue from which the cell was derived (versus normal cells). In addition, as more data is accumulated, the mean expression value can be revised, providing improved relative expression values based on accumulated data. Expression data from normal cells provides a means for grading the severity of the lupus disease state.

In another embodiment, expression of a marker is assessed by preparing genomic DNA or mRNA/cDNA (i.e., a transcribed polynucleotide) from cells in a subject sample, and by hybridizing the genomic DNA or mRNA/cDNA with a reference polynucleotide which is a complement of a polynucleotide comprising the marker, and fragments thereof. cDNA can, optionally, be amplified using any of a variety of polymerase chain reaction methods prior to hybridization with the reference polynucleotide. Expression of one or more markers can likewise be detected using quantitative PCR (QPCR) to assess the level of expression of the marker(s). Alternatively, any of the many known methods of detecting mutations or variants (e.g., single nucleotide polymorphisms, deletions, etc.) of a marker of the invention can be used to detect occurrence of a mutated marker in a subject.

In a related embodiment, a mixture of transcribed polynucleotides obtained from the sample is contacted with a substrate having fixed thereto a polynucleotide complementary to or homologous with at least a portion (e.g., at least 7, at least 10, at least 15, at least 20, at least 25, at least 30, at least 40, at least 50, at least 100, at least 500, or more nucleotide residues) of a marker of the invention. If polynucleotides complementary to or homologous with a marker of the invention are differentially detectable on the substrate (e.g., detectable using different chromophores or fluorophores, or fixed to different selected positions), then the levels of expression of a plurality of markers can be assessed simultaneously using a single substrate (e.g., a “gene chip” microarray of polynucleotides fixed at selected positions). When a method of assessing marker expression is used which involves hybridization of one nucleic acid with another, the hybridization can be performed under stringent hybridization conditions.

In another embodiment, a combination of methods to assess the expression of a marker is utilized.

Because the compositions, kits, and methods of the invention rely on detection of a difference in expression levels of one or more markers of the invention, in certain embodiments the level of expression of the marker is significantly greater than the minimum detection limit of the method used to assess expression in at least one of a biological sample from a subject with lupus or a reference.

### Nucleic Acid Molecules and Probes

One aspect of the invention pertains to isolated nucleic acid molecules that correspond to one or markers of the invention, including nucleic acids which encode a polypeptide corresponding to one or more markers of the invention or a portion of such a polypeptide. The nucleic acid molecules of the invention include those nucleic acid molecules which reside in genomic regions identified herein. Isolated nucleic acid molecules of the invention also include nucleic acid molecules sufficient for use as hybridization probes to identify nucleic acid molecules that correspond to a marker of the invention, including nucleic acid molecules which encode a polypeptide corresponding to a marker of the invention, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded; in certain embodiments the nucleic acid molecule is double-stranded DNA.

An “isolated” nucleic acid molecule is one which is separated from other nucleic acid molecules which are present in the natural source of the nucleic acid molecule. In certain embodiments, an “isolated” nucleic acid molecule is free of sequences (such as protein-encoding sequences) which naturally flank the nucleic acid (i.e., sequences located at the 5′ and 3′ ends of the nucleic acid) in the genomic DNA of the organism from which the nucleic acid is derived.

The language “substantially free of other cellular material or culture medium” includes preparations of nucleic acid molecule in which the molecule is separated from cellular components of the cells from which it is isolated or recombinantly produced. Thus, nucleic acid molecule that is substantially free of cellular material includes preparations of nucleic acid molecule having less than about 30%, less than about 20%, less than about 10%, or less than about 5% (by dry weight) of other cellular material or culture medium.

If so desired, a nucleic acid molecule of the present invention, e.g., the marker gene products identified herein, can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR amplification techniques. The nucleic acid molecules so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

Probes based on the sequence of a nucleic acid molecule of the invention can be used to detect transcripts (e.g., mRNA) or genomic sequences corresponding to one or more markers of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

### Polypeptide Detection

Methods to measure biomarkers of this invention, include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF) mass spectrometry, microcytometry, microarray, microscopy, fluorescence activated cell sorting (FACS), flow cytometry, laser scanning cytometry, hematology analyzer and assays based on a property of the protein including but not limited to DNA binding, ligand binding, or interaction with other protein partners.

The activity or level of a marker protein can also be detected and/or quantified by detecting or quantifying the expressed polypeptide. The polypeptide can be detected and quantified by any of a number of means well known to those of skill in the art. These can include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, immunohistochemistry and the like. A skilled artisan can readily adapt known protein/antibody detection methods for use in determining the expression level of one or more biomarkers in a serum sample.

Another agent for detecting a polypeptide of the invention is an antibody capable of binding to a polypeptide corresponding to a marker of the invention, e.g., an antibody with a detectable label. Antibodies can be polyclonal or monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In another embodiment, the antibody is labeled, e.g., a radio-labeled, chromophore-labeled, fluorophore-labeled, or enzyme-labeled antibody. In another embodiment, an antibody derivative (e.g., an antibody conjugated with a substrate or with the protein or ligand of a protein-ligand pair {e.g., biotin-streptavidin}), or an antibody fragment (e.g., a single-chain antibody, an isolated antibody hypervariable domain, etc.) which binds specifically with a protein corresponding to the marker, such as the protein encoded by the open reading frame corresponding to the marker or such a protein which has undergone all or a portion of its normal post-translational modification, is used.

Proteins from cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

In one format, antibodies, or antibody fragments, can be used in methods such as Western blots or immunofluorescence techniques to detect the expressed proteins. In such uses, one can immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

In another embodiment, the polypeptide is detected using an immunoassay. As used herein, an immunoassay is an assay that utilizes an antibody to specifically bind to the analyte. The immunoassay is thus characterized by detection of specific binding of a polypeptide to an anti-antibody as opposed to the use of other physical or chemical properties to isolate, target, and quantify the analyte.

The polypeptide is detected and/or quantified using any of a number of well recognized immunological binding assays (see, e.g., U.S. Pat. Nos. 4,366,241; 4,376,110; 4,517,288; and 4,837,168). For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

In another embodiment, the polypeptide is detected and/or quantified using Luminex™ assay technology. The Luminex™ assay separates tiny color-coded beads into e.g., distinct sets that are each coated with a reagent for a particular bioassay, allowing the capture and detection of specific analytes from a sample in a multiplex manner. The Luminex™ assay technology can be compared to a multiplex ELISA assay using bead-based fluorescence cytometry to detect analytes such as biomarkers.

The invention also encompasses kits for detecting the presence of a polypeptide or nucleic acid corresponding to a marker of the invention in a biological sample, e.g., a sample containing tissue, whole blood, serum, plasma, buccal scrape, saliva, cerebrospinal fluid, urine, stool, and bone marrow. Such kits can be used to determine if a subject is suffering from or is at increased risk of developing lupus. For example, the kit can comprise a labeled compound or agent capable of detecting a polypeptide or an mRNA encoding a polypeptide corresponding to a marker of the invention in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

The invention thus includes a kit for assessing the disease progression of a subject having lupus. Suitable reagents for binding with a polypeptide corresponding to a marker of the invention include antibodies, antibody derivatives, antibody fragments, and the like. Suitable reagents for binding with a nucleic acid (e.g., a genomic DNA, an mRNA, a spliced mRNA, a cDNA, or the like) include complementary nucleic acids. For example, the nucleic acid reagents can include oligonucleotides (labeled or non-labeled) fixed to a substrate, labeled oligonucleotides not bound with a substrate, pairs of PCR primers, molecular beacon probes, and the like.

The kit of the invention can optionally comprise additional components useful for performing the methods of the invention. By way of example, the kit can comprise fluids (e.g., SSC buffer) suitable for annealing complementary nucleic acids or for binding an antibody with a protein with which it specifically binds, one or more sample compartments, an instructional material which describes performance of a method of the invention, a reference sample for comparison of expression levels of the biomarkers described herein, and the like.

A kit of the invention can comprise a reagent useful for determining protein level or protein activity of a marker.

### Lupus Therapeutic Agents, Compositions and Administration

There are several medications presently used to modify the course of lupus. Such agents include, but are not limited to, nonsteroidal anti-inflammatory drugs (NSAID); antimalarials, including, for example, hydroxychloroquine; corticosteroids, including, for example, glucocorticoids; immunosuppressants, including, for example, azathioprine, mycophenolate mofetil, or methotrexate; and intravenous immunoglobulins. In some embodiments, any combination of medications presently used to modify the course of lupus comprises a first lupus therapy.

The invention described herein provides for the use of second or alternative therapies for the treatment of lupus when a first lupus therapy is less responsive or shows disease progression when treated with a first therapy. Such agents include, but are not limited to, an anti-TWEAK antibody; an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; or an anti-BAFF antibody.

### Nonsteroidal Anti-Inflammatory Drugs

One known therapy for lupus includes treatment with nonsteroidal anti-inflammatory drugs (NSAIDs). NSAIDs are anti-inflammatory agents that are not steroids. In addition to anti-inflammatory actions, they have analgesic, antipyretic, and platelet-inhibitory actions. They are used primarily in the treatment of chronic arthritic conditions and certain soft tissue disorders associated with pain and inflammation. They act by blocking the synthesis of prostaglandins by inhibiting cyclooxygenase, which converts arachidonic acid to cyclic endoperoxides, precursors of prostaglandins. Inhibition of prostaglandin synthesis accounts for their analgesic, antipyretic, and platelet-inhibitory actions; other mechanisms may contribute to their anti-inflammatory effects. Certain NSAIDs also may inhibit lipoxygenase enzymes or phospholipase C or may modulate T-cell function. NSAIDs include, but are not limited to, acid (e.g., aspirin), ibuprofen (Motrin), naproxen (Naprosyn), indomethacin (Indocin), nabumetone (Relafen), tolmetin (Tolectin), diclofenac sodium (Voltaren), diflunisal (Dolobid), and any other embodiments that comprise a therapeutically equivalent active ingredient(s) and formulation thereof.

Methods for making NSAIDs are known in the art.

Dosages of NSAIDs to administer can be determined by one of skill in the art, and include clinically acceptable amounts to administer based on the specific NSAIDs used. For example, indomethacin is typically administered orally at 25 to 50 mg, 2 to 4 times daily. Other forms of NSAIDs, specifically naproxen, are administered, for example, orally at 440 to 1,500 mg daily.

### Antimalarials

Antimalarials are effective in controlling lupus arthritis, skin rashes, mouth ulcers, and other symptoms such as fatigue and fever. They are used to manage less serious forms of SLE in which no organs have been damaged. Antimalarials are not used to manage more serious, systemic forms of SLE that affect the organs.

Antimalarials were first developed during World War II because quinine, the standard treatment for malaria, was in short supply. Investigators discovered antimalarials could also be used to treat the joint pain that occurs with rheumatoid arthritis. Subsequent use of antimalarials showed that they are effective in controlling lupus arthritis, skin rashes, mouth ulcers, fatigue, and fever. Antimalarials are also very effective in the treatment of DLE.

The anti-inflammatory action of antimalarials is not well understood. In some patients who take antimalarials, the total daily dose of corticosteroids can be reduced. Antimalarials also affect platelets to reduce the risk of blood clots and lower plasma lipid levels. Antimalarials include but are not limited to hydroxychloroquine sulfate (Plaquenil) and chloroquine (Aralen).

Methods for making antimalarials are known in the art.

Dosages of antimalarials to administer can be determined by one of skill in the art, and include clinically acceptable amounts to administer based on the specific antimalarials used. For example, hydroxychloroquine is typically administered orally at 200 mg to 400 mg, 1 to 2 times daily. Other forms of antimalarials, specifically chloroquine, are administered, for example, orally at 250 mg daily.

### Corticosteroids

Corticosteroids reduce inflammation in various tissues of the body. They are used to treat many of the symptoms of lupus that result from inflammation. They can be administered orally, by injection, or topically.

Corticosteroids are hormones secreted by the cortex of the adrenal gland. SLE patients with symptoms that do not improve or who are not expected to respond to NSAIDs or antimalarials may be given a corticosteroid. Although corticosteroids have potentially serious side effects, including changes in appearance (such as acne or increased facial hair); development of a round or moon-shaped face; thin, fragile skin that bruises easily; movement of body fat to the trunk; mood changes; personality changes; irritability; agitation; or depression; they are highly effective in reducing inflammation, relieving muscle and joint pain and fatigue, and suppressing the immune system.

They are also useful in controlling major organ involvement associated with SLE. Corticosteroids are given in much higher doses than the body produces and act as potent therapeutic agents.

Once the symptoms of lupus have responded to treatment, the dose is usually tapered until the lowest possible dose that controls disease activity is achieved. Patients must be monitored carefully during this time for flares or recurrence of joint and muscle pain, fever, and fatigue that can result when the dosage is lowered. Some patients may require corticosteroids only during active stages of the disease; those with severe disease or more serious organ involvement may need long-term treatment.

Corticosteroids include but are not limited to prednisone (Orasone®, Meticorten®, Deltasone®, Cortan®, Sterapred®), a synthetic corticosteroid, most often used to treat lupus, hydrocortisone (Cortef®, Hydrocortone®), methylprednisolone (Medrol®), and dexamethasone (Decadron®).

Methods for making corticosteroids are known in the art. Dosages of corticosteroids to administer can be determined by one of skill in the art, and include clinically acceptable amounts to administer based on the specific corticosteroids used and the severity of the disease. For example, prednisone is typically administered orally at 40 to 80 milligrams a day when there is involvement of the heart Involvement of the heart, lungs, kidneys, liver or blood. Severe flare-ups of non-organ threatening disease are treated with moderate doses of 20 to 40 milligrams a day. Chronic, mild, non-organ-threatening disease responds to daily doses of 2 to 20 milligrams a day.

### Immunosuppressants

Immunosuppressive drugs suppress immune function. They can suppress any arm of the immune system, like T cells or B cells, they can suppress transplanted organ rejection, the making of antibodies, or the attack of certain cells, viruses or tumours. In people with lupus, the immune system mistakenly attacks the body's own tissues. By downregulating the immune system and reducing the production of antibodies, the damage done to the body, as a result of immune complexes, is reduced. People taking immunosuppressive drugs are at an increased risk for infection.

Immunosuppressive medications are used to control more serious lupus activity that affects major organs, including the kidney, brain, cardiovascular system, and lungs. Sometimes immunosuppressive medications are given in addition to or instead of steroid therapy to lower the dose of steroids needed and thus spare some of the undesirable side effects of steroid therapy. For this reason, these drugs are sometimes called “steroid-sparing” medications or “adjuvant” (helping) drugs. Steroid-sparing drugs usually have a two-fold benefit, since they often reduce or eliminate the need for steroids while also improving lupus symptoms.

Methods for making immunosuppressive drugs are known in the art.

Immunosuppresants include, but are not limited to, azathioprine, cyclophosphamide, chlorambucil, mycophenolate mofetil and methotrexate.

Dosages of immunosuppresants to administer can be determined by one of skill in the art, and include clinically acceptable amounts to administer based on the specific immunosuppresants used. For example, methotrexate is typically administered orally at 7.5 milligrams once a week.

### Intravenous Immunoglobulins

Intravenous immunoglobulin (IVIG) is a blood product administered intravenously. It contains the pooled, polyvalent, IgG antibodies extracted from the plasma of over one thousand blood donors. Methods for making immunosuppressive drugs are known in the art.

Dosages of IVIG to administer can be determined by one of skill in the art. IVIG's effects last between 2 weeks and 3 months. In the case of patients with autoimmune disease, such as lupus, IVIG is administered at a high dose (generally 1-2 grams IVIG per kg body weight) to attempt to decrease the severity of the autoimmune disease.

### Anti-TWEAK Antibody

Therapeutic approaches for inhibiting TWEAK (also known as tumor necrosis factor ligand superfamily member 12 or TNF-related weak inducer of apoptosis) in lupus nephritis are based on, at least in part, multiple rationales, including blocking the binding of TWEAK to Fn14 (fibroblast growth factor-inducible 14), an anti-TWEAK antibody, e.g., BIIB023, attenuates TWEAK/Fn14 signaling and the downstream cellular responses of this signaling cascade; TWEAK induces the expression of proinflammatory mediators in both mesangial cells and podocytes, as well as in renal tubules, which may promote glomerulonephritis and tubulointerstitial inflammation. Since inflammation is considered to be an important mediator of tissue damage, TWEAK may promote tissue damage in lupus nephritis by promoting the recruitment of inflammatory infiltrates. TWEAK also acts in concert with other cytokines to promote renal tubular cell death. Thus, TWEAK may play an important pathogenic role in glomerulonephritis by promoting a local inflammatory environment and inducing tissue damage leading to progression to both glomerulo- and tubulointerstitial fibrosis.

Because TWEAK may promote multiple pathogenic activities locally in the kidney, it represents a promising target for therapeutic intervention of lupus nephritis in particular. Inhibition of the TWEAK/Fn14 pathway with anti-TWEAK monoclonal antibodies has proven effective in multiple animal models of inflammatory diseases, suggesting that TWEAK blockade by anti-TWEAK antibodycan be clinically beneficial in lupus nephritis. (Michaelson, J S et al. (2012) J. Autoimmun. 39(3): 130-142).

### Anti-CD40 and Anti-CD40L Antibodies

The interaction between CD40 on B cells and CD40 ligand (CD40L) on activate T helper cells is required for normal antibody production. Monoclonal antibodies that block CD40:CD40L interaction would block B cell differentation and could induce tolerance. A monoclonal antibody anti-CD40L has proved to be efficacious in reducing anti-DNA antibodies production, controlling renal disease and prolonging survival in New Zealand Black x New Zealand White lupus-prone mice (Early, G S et al. (1996) The Journal of Immunology 157(7): 3159-3164).

Preliminary data from phase I single-dose, dose-escalating trial with a humanised anti-CD40L monoclonal antibody, in 23 patients with SLE have shown that the drug is well tolerated, with mild to moderate side effects, mainly asthenia, dizziness, nausea and headache.

Exemplary anti-CD40L antibodies are disclosed, e.g., in WO 05/003175, US 2013/0045219. Modulators, e.g., interruptors, of CD40:CD154 interactions are disclosed in, e.g., WO 2002/018446, WO 2001/0303386.

### Anti-CD20 Antibodies (Rituximab)

The anti-CD20 antibody binds to CD20. CD20 is widely expressed on B from early pre-B cells to later in differentiation, but it is absent on terminally differentiated plasma cells. CD20 does not shed, modulate or internalize. Although the function of CD20 is unknown, it may play a role in Ca2+ influx across plasma membranes, maintaining intracellular Ca2+ concentration and allowing activation of B cells. Anti-CD20 antibody destroys B cells and is therefore used to treat diseases which are characterized by excessive numbers of B cells, overactive B cells, or dysfunctional B cells, such as lupus, which is characterized by an overabundance of autoantibodies produced by B cells. (Eisenberg R. (2006) Endocr Metab Immune Disord Drug Targets (4):345-50)

### Anti-Interferon Antibody (Sifalimumab)

The cytokine family of type I interferons (IFNs), and especially the IFNα subtypes, are implicated as important players in lupus pathogenesis. This is supported by several observations. IFNα treatment is sometimes associated with the development of autoantibodies and even SLE-like features. In patients with SLE, high type I IFN or IFN-driven chemokine levels are associated with greater disease activity. Genetic polymorphisms of several components of the IFN signaling pathway have been associated with an increased risk of SLE. Furthermore, mice deficient in the IFNα/β receptor have been shown to exhibit reduced signs and symptoms of SLE, and the IFNα kinoid vaccine prevents clinical manifestations in a lupus flare model. Therefore, IFNα subtypes have been identified as a potential target for drug development in SLE.

Sifalimumab is a human anti-IFNα monoclonal antibody that binds to and specifically neutralizes most IFNα subtypes, preventing signaling through the type I IFN receptor. In a phase Ia study of patients with SLE, single doses of sifalimumab were shown to have linear, dose-proportional pharmacokinetics (PK) and dose-dependent inhibition of the type I IFN-inducible gene signature (Yao, Y et al. (2009) Arthritis Rheum 60: 1785-96; Merrill, J T et al. (2011) Ann Rheum Dis 70: 1905-13).

### Rapamycin

Rapamycin, is a macrolide produced by the bacteria Streptomyces hygroscopicus. It has immunosuppressant functions in humans. It prevents activation of T cells and B cells by inhibiting their response to interleukin-2 (IL-2).

Research has documented that rapamycin improves disease activity and dependence on prednisone in systemic lupus erythematosus (SLE) patients resistant or intolerant to immunosuppressant medications. Rapamycin acts through blocking the activation of its molecular target, the mechanistic target of rapamycin complex 1 (mTORC1). The activation of mTORC1, which is associated with suppression of mTORC2, results in the expansion of pro-inflammatory CD4-CD8-double-negative (DN) T lymphocytes. These DN T cells produce inflammatory cytokines, interleukin-4 (IL-4) and interleukin-17, and they exhibit predisposition to pro-inflammatory cell death through necrosis. Increased IL-4 production is responsible for activation of autoantibody-producing B lymphocytes in SLE. Rapamycin also blocks disease in lupus-prone mice by reversing the activation of mTORC1 (Lai, Z W et al. (2013) J Immunol. 191(5):2236-46).

### Arsenic Trioxide

Over the last 17 years, clinical trials conducted worldwide have demonstrated the efficacy of arsenic trioxide (As₂O₃) in the treatment of relapsed acute promyelocytic leukemia (APL). Currently, the role of As₂O₃ in front-line therapy is under investigation. Arsenic has been used in medicine for more than 2400 years for a variety of ailments including ulcers, the plague, and malaria.¹ In 1878, potassium arsenite was reported to have an anti-leukemic effect and was used for this purpose in the late 19th and early 20th centuries until it was replaced by busulfan in the 1950s. In the modern era, interest in arsenic as a chemotherapy was rekindled after it was identified as an active ingredient in traditional medicines in China.

As₂O₃ affects multiple cellular functions via different molecular targets. Although the fundamental mechanism is the favorable chemical reaction between arsenic and thiol groups within a protein, the final outcome depends on the cell type as well as the dose and duration of arsenite exposure. For example, in certain types of cells cells, As₂O₃ at low concentrations (<0.5 μM) induces differentiation; at higher concentrations (0.5-2.0 μM) it causes apoptosis (Ashkan, E et al. (2010) Blood Rev. 24(4-5): 191-199).

MRL/lpr mice develop a human lupus like syndrome and, as in autoimmune lymphoproliferative syndrome (ALPS), massive lymphoproliferation due to inactivation of Fas-mediated apoptosis. arsenic trioxide (As2O3) is able to achieve quasi-total regression of antibody- and cell-mediated manifestations in MRL/lpr mice (Bobé, P et al. (2006) Blood 108(13):3967-75).

### 5-chloro-N-ethyl-4-hydroxy-1-methyl-2-oxo-N-phenyl-1,2-dihydroquinoline-3-carboxamide (Laquinimod)

Laquinimod reduces infiltration of inflammatory cells into the spinal cord in experimental autoimmune encephalomyelitis. Activated monocyte/macrophages infiltrate the kidneys during nephritis in systemic lupus erythematosus (SLE) (Lourenco, E V et al. (2014) Arthritis Rheumatol. 66(3):674-85). Results of a Phase 2 trial showed that Laquinimod improved kidney function in patients with lupus nephritis.

### Anti-BDCA2 Antibody

CD303 (BDCA-2) is a plasmacytoid dendritic cell-specific antigen present in lymphoid and nonlymphoid tissue and contribute substantially to both innate and adaptive immunity. Several monoclonal antibodies have been identified that recognize this antigen (described in, e.g., Dzionek, A. et al. (2001) J. Exp. Med. 194(12):1823-34). Molecular cloning of BDCA-2 revealed that BDCA-2 is a novel type II C-type lectin, which shows 50.7% sequence identity at the amino acid level to its putative murine ortholog, the murine dendritic cell-associated C-type lectin 2. Anti-BDCA-2 monoclonal antibodies are rapidly internalized and efficiently presented to T cells, indicating that BDCA-2 could play a role in ligand internalization and presentation. Furthermore, ligation of BDCA-2 potently suppresses induction of interferon alpha/beta production in plasmacytoid dendritic cells, presumably by a mechanism dependent on calcium mobilization and protein-tyrosine phosphorylation by src-family protein-tyrosine kinases. Production of interferon alpha/beta by plasmacytoid dendritic cells is considered to be an important pathophysiological factor in systemic lupus erythematosus; triggering of BDCA-2 has been suggested as therapeutic strategy for blocking production of interferon alpha/beta in systemic lupus erythematosus patients.

### Anti-BAFF Antibody (Belimumab)

Belimumab is a human monoclonal antibody that inhibits B-cell activating factor (BAFF), also known as B-lymphocyte stimulator (BLyS) described herein. B cells are responsible for part of the normal immune response, and also for the over-aggressive immune response and overproduction of autoantibodies in autoimmune diseases like lupus. B-cell activating factor (BAFF), also called B-lymphocyte stimulator (BLyS), is required for the development and survival of B cells. In lupus, BAFF is overexpressed. Researchers theorize that BAFF overexpression causes autoimmune B cell proliferation and survival, which causes lupus. Belimumab is a human antibody that binds to BAFF, preventing BAFF from binding to B cells. Without the survival factor BAFF, B cells commit suicide, and no longer contribute to the autoimmune damage of lupus. (Kaveri, S V et al. (2010) N Engl J Med 363 (11): 1080-1082).

The efficacy and safety of Belimumab was demonstrated in 2 Phase III randomized, controlled studies. The 2 studies had a total of 1,684 patients, with scores of ≧6 on the SELENA-SLEDAI assessment. They were divided into a placebo and 2 dosage groups of Belimumab, in addition to standard therapy. The primary end point was a reduction of ≧4 on the SELENA-SLEDAI assessment, and several other factors, at 52 weeks. Belimumab significantly improved the response rate, reduced disease activity and severe flares, and was well tolerated. 58% had SELENA-SLEDAI scores reduced by ≧4 points during 52 weeks with Belimumab 10 mg/kg compared to 46% with placebo.

### Therapeutic Methods

“Treat,” “treatment,” and other forms of this word refer to the administration of a therapy (e.g., a lupus therapy), alone or in combination with one or more other lupus therapies, to a subject, e.g., a lupus patient, to impede progression of lupus, to treat a flare, to restore function, to extend the expected survival time of the subject and or reduce the need for medical interventions (e.g., hospitalizations). In those subjects, treatment can include, but is not limited to, inhibiting or reducing one or more symptoms such as which can be treated with the methods described herein or managed using symptom management therapies, include, but are not limited to, achy joints/arthralgia, fever of more than 100° F./38° C., arthritis/swollen joints, prolonged or extreme fatigue, skin rashes, anemia, kidney involvement, pain in the chest on deep breathing/pleurisy, butterfly-shaped rash across the cheeks and nose, sun or light sensitivity/photosensitivity, hair loss, blood clotting problems, Raynaud's phenomenon/fingers turning white and/or blue in the cold, seizures, mouth or nose ulcers, and any combination thereof; prolonging survival, or prolonging progression-free survival, and/or enhanced quality of life and improving established disease.

As used herein, unless otherwise specified, the terms “prevent,” “preventing” and “prevention” contemplate an action that occurs before a subject begins to suffer from the a lupus flare or progression and/or which inhibits or reduces the severity of the disease.

As used herein, and unless otherwise specified, the terms “manage,” “managing” and “management” encompass preventing the progression of lupus symptoms in a patient who has already suffered from the disease, and/or lengthening the time that a patient who has suffered from a lupus flare remains in remission. The terms encompass modulating the threshold, development and/or duration of lupus, or changing the way that a patient responds to the disease.

As used herein, and unless otherwise specified, a “therapeutically effective amount” of a compound is an amount sufficient to provide a therapeutic benefit in the treatment or management of lupus, or to delay or minimize one or more symptoms associated with lupus. A therapeutically effective amount of a compound means an amount of therapeutic agent, alone or in combination with other therapeutic agents, which provides a therapeutic benefit in the treatment or management of lupus. The term “therapeutically effective amount” can encompass an amount that improves overall therapy, reduces or avoids symptoms or causes of the disease, or enhances the therapeutic efficacy of another therapeutic agent.

As used herein, and unless otherwise specified, a “prophylactically effective amount” of a compound is an amount sufficient to prevent relapse of lupus after a flare, or one or more symptoms associated with the disease, or prevent its recurrence after a flare. A prophylactically effective amount of a compound means an amount of the compound, alone or in combination with other therapeutic agents, which provides a prophylactic benefit in the prevention of lupus relapse after a flare. The term “prophylactically effective amount” can encompass an amount that improves overall prophylaxis or enhances the prophylactic efficacy of another prophylactic agent.

As used herein, the term “patient” or “subject” refers to a mammal, typically a human (i.e., a male or female of any age group, e.g., a pediatric patient (e.g., infant, child, adolescent) or adult patient (e.g., young adult, middle-aged adult or senior adult) or other mammal, such as a primate (e.g., cynomolgus monkey, rhesus monkey); commercially relevant mammals such as cattle, pigs, horses, sheep, goats, cats, and/or dogs; and/or birds, including commercially relevant birds such as chickens, ducks, geese, and/or turkeys, that will be or has been the object of treatment, observation, and/or experiment. When the term is used in conjunction with administration of a compound or drug, then the patient has been the object of treatment, observation, and/or administration of the compound or drug.

The methods described herein permit one of skill in the art to identify a monotherapy that a lupus patient is most likely to respond to, thus eliminating the need for administration of multiple therapies to the patient to ensure that a therapeutic effect is observed. However, in one embodiment, combination treatment of an individual with lupus is also disclosed.

### Combination Therapy

It will be appreciated that any lupus therapy as described above and herein, can be administered in combination with one or more additional therapies to treat and/or reduce the symptoms of lupus described herein, particularly to treat patients with moderate to severe disease. The pharmaceutical compositions can be administered concurrently with, prior to, or subsequent to, one or more other additional therapies or therapeutic agents. In general, each agent will be administered at a dose and/or on a time schedule determined for that agent. In will further be appreciated that the additional therapeutic agent utilized in this combination can be administered together in a single composition or administered separately in different compositions. The particular combination to employ in a regimen will take into account compatibility of the pharmaceutical composition with the additional therapeutically active agent and/or the desired therapeutic effect to be achieved. In general, it is expected that additional therapeutic agents utilized in combination be utilized at levels that do not exceed the levels at which they are utilized individually. In some embodiments, the levels utilized in combination will be lower than those utilized individually.

The methods provided herein are useful for identifying subjects as disease progressing. In some embodiments, the disease progression value as described herein, is acquired for a subject with a progressive form of lupus at a single time point; or at baseline and 1 year after the initiation of therapy; or at the time there is a change in therapy and 1 year post the change in therapy). In some embodiments, a subject identified as disease progressing based on a change in the disease progression value during longitudinal follow up with recurrent periodic assessments. In some embodiments, a subject identified as disease progressing based on an initial disease progression value and a SLEDA scale value obtained during longitudinal follow up with recurrent periodic assessments.

The methods provided herein are also useful for identifying subjects that are more likely to respond to, or are in need of, an alternative therapy, e.g., anti-BAFF, anti. In some embodiments, a disease progression value is measured prior to the initiation of an alternative therapy, and based solely on the disease progression value or based on the disease progression value in combination with other factors (e.g., presence or absence or degree of cognitive impairment associated with MS); an alternative therapy is recommended or administered. The methods provided herein are also useful for identifying subjects that are not in need of an alternative therapy, e.g., an anti-TWEAK antibody; an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; or an anti-BAFF antibody. In some embodiments, a disease progression value is measured prior to the initiation of a therapy, and based solely on the disease progression value or based or the disease progression value in combination with other factors (e.g., presence/absence or degree of symptoms associated with lupus); an alternative therapy, e.g., an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; or an anti-BAFF antibody, is recommended or administered.

The methods provided herein are also useful for identifying subjects that are more likely to respond to or are in need of an alternative therapy, e.g., an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; or an anti-BAFF antibody. In some embodiments, a disease progression value is measured, and based solely on the disease progression value or based on the disease progression value in combination with other factors (e.g., presence or absence or degree of symptoms associated with lupus); an alternative therapy is recommended or administered.

The methods provided herein are also useful for identifying subjects that are not more likely to respond to or are not in need of an alternative therapy, e.g., an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; or an anti-BAFF antibody. In some embodiments, a disease progression value is measured, and based solely on the disease progression value or based on the disease progression value in combination with other factors (e.g., presence or absence or degree of symptoms associated with lupus); an alternative therapy is not recommended or withheld.

The methods described herein can also be used to monitor a response to a therapy. Such methods are useful for detection of tolerance to a therapy, ineffectiveness of a therapy, or a positive response to a therapy. In some embodiments, a disease progression value is measured at least 3 months, at least 4 months, at least 5 months, at least 6 months, at least 7 months, at least 8 months, or at least 1 year after initiation of a therapy.

In some embodiments, the disease progression value is compared to a reference value or cut-off value. For example, a cut off value can be determined that represents a diseases progression status; any value falling above the cut-off value are classified as a disease progressing. In another example, a cut-off value can be determined that represents a particular therapy should be administered, e.g., an alternative therapy, e.g., an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; or an anti-BAFF antibody.

In some embodiments, a change in the disease progression value is determined. In one embodiment, the change in the disease progression value is determined by comparing the disease progression value acquired for a subject with lupus at two or more timepoints (e.g., at baseline and 6 months after initiation of a therapy; at baseline and 12 months after initiation of a therapy; 6 and 12 months after initiation of a therapy; at the time of a change in a therapy and 6 months post the change in a therapy; or at the time of a change in a therapy and 6 months and 12 months post the change in a therapy).

The present invention also pertains to the field of predictive medicine in which diagnostic assays, pharmacogenomics, and monitoring clinical trials are used for predictive purposes to thereby treat an individual prophylactically. Accordingly, one aspect of the present invention relates to methods for determining a disease progression value, in order to determine whether an individual having lupus or at risk of developing lupus should be classified as disease progressing. Accordingly, one aspect of the present invention relates to assays for determining a disease progression value, in order to determine whether an individual having lupus or at risk of developing lupus should be administered an alternative therapy, e.g., an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; or an anti-BAFF antibody. Accordingly, one aspect of the present invention relates to assays for determining a disease progression value, in order to determine whether an individual having lupus or at risk of developing lupus should be administered a first therapy, e.g., a nonsteroidal anti-inflammatory drug (NSAID); an antimalarial, including, for example, hydroxychloroquine; a corticosteroid, including, for example, a glucocorticoid; an immunosuppressant, including, for example, azathioprine, mycophenolate mofetil, or methotrexate; and/or an intravenous immunoglobulin, or an alternative therapy, e.g., an anti-TWEAK antibody; an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; and/or an anti-BAFF antibody.

In one aspect, the invention is drawn to a method for determining whether a subject is in need of a lupus therapy. In another aspect, the method is drawn to selecting a lupus therapy. In another aspect, the invention is drawn to a method of administering the lupus therapy. In another aspect the, the invention is drawn to a method of altering dosing of the lupus therapy. In another aspect, the invention is drawn to a method of altering a schedule or a time course of the lupus therapy. In still another aspect, the invention is drawn to a method of administering an alternative lupus therapy.

In certain embodiments, the method comprises acquiring a disease progression value from a subject as described herein, and determining whether the subject is in need of a lupus therapy. In certain embodiments, the method comprises acquiring a disease progression value from a subject as described herein and selecting; altering composition of; altering dosage of; altering dosing schedule of; a lupus therapy.

In some embodiments, the methods involve evaluation of a subject e.g., a patient, a patient group or a patient population, e.g., a patient who has been diagnosed with or is suspected of having lupus, e.g., presents with symptoms of lupus, to acquire a disease progression value as described herein.

In some embodiments, the results of the acquisition of the disease progression value and the interpretation thereof, are predictive that a patient is a disease progressor. In some embodiments, the results of the acquisition of the disease progression value and the interpretation thereof, are predictive of the patient's need for or response to treatment with an alternative therapy, e.g., an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; or an anti-BAFF antibody. In some embodiments, the results of the acquisition of the disease progression value and the interpretation thereof, are predictive of the patient's need for or response to treatment with a first therapy, e.g., a nonsteroidal anti-inflammatory drug (NSAID); an antimalarial, including, for example, hydroxychloroquine; a corticosteroid, including, for example, a glucocorticoid; an immunosuppressant, including, for example, azathioprine, mycophenolate mofetil, or methotrexate; and/or an intravenous immunoglobulin, or an alternative therapy, e.g., an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; or an anti-BAFF antibody. According to the present invention, a disease progression value described herein, can be indicative that treatment with an alternative therapy, e.g., an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; or an anti-BAFF antibody, or a combination therapy should be recommended or administered.

In yet another embodiment, the disease progression value is assessed at pre-determined intervals, e.g., a first point in time and at least at a subsequent point in time. In one embodiment, a time course is measured by determining the time between significant events in the course of a patient's disease, wherein the measurement is predictive of whether a patient has a long time course. In another embodiment, the significant event is the progression from primary diagnosis to death. In another embodiment, the significant event is the progression from primary diagnosis to worsening disease. In another embodiment, the significant event is the progression from primary diagnosis to flare. In another embodiment, the significant event is the progression from flare to death. In certain embodiments, the time course is measured with respect to one or more of overall survival rate, time to progression and/or using the SLEDA or other assessment criteria.

The methods described herein can be used in any subject having lupus, including but not limited to, systemic lupus erythematosus (SLE) (e.g., lupus nephritis), cutaneous lupus erythematosus (CLE) (e.g., acute cutaneous lupus erythematosus (ACLE), subacute cutaneous lupus erythematosus (SCLE), intermittent cutaneous lupus erythematosus, and chronic cutaneous lupus), drug-induced lupus, and neonatal lupus.

### Exemplary Computer System

Various computer systems can be specially configured to leverage information returned on potential Lupus disease state indicators. In some embodiments, the computer system can determine and present information on confidence levels associated with various biomarkers and/or indicators for Lupus. For example, the computer systems can evaluate whether a test conducted on a patient indicates an expected increase in a SLEDAI score and along with a degree of confidence associated with the expected increase. In further examples, the system can provide an indication and/or recommendation on increasing the degree of confidence associated with the expected increase in SLEDAI. For example, the system can be configured to evaluate any tests and tested biomarkers and/or indicators of Lupus that have been performed for a subject against another characteristic identified as independent and/or additive of the existing data. The system can determine when an additional biomarker and/or indicator (e.g., gene signature) would increase confidence associated with, for example, anticipated SLEDAI change. The system can recommend testing of any identified characteristic accordingly.

In some embodiments, an interactive system for identification, assessment and/or treatment of a subject having lupus can be provided. According to one embodiment, the system can be configured to accept user input regarding degree of confidence of a patient assessment. Responsive to the user entered degree of confidence, the system can determine test characteristics to include in an evaluation model. In one example, the system includes specification of independent indicators for disease activity in a patient population. The system can be configured to estimate a degree of confidence in a determination of disease activity or a prediction of future disease activity based on what independent indicators are used. The system can be further configured to determined and/or recommend various combinations of the determined independent indicators to improve a degree of confidence in an evaluation.

According to another aspect, a computer system can be specially configured to evaluate indicators for Lupus. The system can be configured to generate a multivariate model, wherein the multivariate model excludes correlated indicators. In some examples, the system can be configured to identify correlated indicators responsive to evaluating returned test results within a patient population having one or more of the indicators. For example, the system can execute regression model analysis to control for various parameters, including, for example, patient age, race, sex, and the presence of other indicators. Responsive to eliminating correlated indicators, the system can generate a model of one or more independent indicators. In some embodiments, the system can be configured to select various combinations of the one or more independent indicators and can further access evaluations (including, for example, evaluating the combination directly) to present information on a confidence level associated with respective selections. The system selected models can be used to generate an expected change in disease activity with the determined confidence level.

According to some embodiments, the system and methods described herein include specification of correlated relationships between biomarkers (e.g., BAFF and IFN-Alpha are specified with a high correlation co-efficient; IFN-Alpha and IFN-Gamma are specified with a high correlation co-efficient; and PC signature is specified with moderate correlation with BAFF and both IFN-Alpha and IFN-Gamma), and can include specification of uncorrelated biomarker relationships (e.g., neutrophil gene signature and TWEAK are not correlated). The system can be configured to build disease evaluation models according to available information on a given patient. For example, the system can be configured to evaluate available information and select a model of disease progression that yields the greatest confidence level for disease state prediction based on available patient data. According to some embodiments, the system can also be configured to determine an optimal model for disease activity (e.g., using a SLEDAI score) as opposed to and/or in conjunction with an optimal model for renal disease activity (e.g., using a renal SLEDAI score).

In further embodiments, the system can generate optimal models based on both measures for disease activity and optimizing an over-all prediction of disease activity (e.g., incorporating one, two, three, or more disease activity components). Each of a variety biomarkers can include specification of relationships between measurement distribution and how increases/decreases in the measurements correlate with scores of disease activity (e.g., for patients in top 55% of IFN-Alpha distribution a one standard deviation increase in IFN-Alpha is associated with 0.61 points in higher mean SLEDAI score; top 15% of neutrophil gene signature expression is associated with 0.62 points higher SLEDAI; for patients in the top 15% of TWEAK, a one standard deviation increase in TWEAK is associated with 0.57 points higher SLEDAI).

In some embodiments, each relationship can also be associated with a confidence score for the association (e.g., IFN-Alpha increase can be associated with a p value of 0.0002; neutrophil gene signature increase is associated with a p value of 0.0090; and TWEAK protein increase is associated with a p value 0.0006). Other examples include correlations between biomarker measurements and increases in a renal component of a SLEDAI score (e.g., for patients in the top 50% of IFN-Alpha a one standard deviation increase in IFN-Alpha is associated with a 0.33 point higher mean renal component of SLEDAI; on average high neutrophil signature is associated with 0.39 higher mean renal component of SLEDAI; for patients with high neutrophil signature a one standard deviation increase in TWEAK is associated with a 0.57 increase in a mean renal component of SLEDAI). In some embodiments, the relationships can also be associated with a confidence score for the association (e.g., IFN-Alpha increase in renal SLEDAI can be associated with a p value of 0.024; neutrophil gene indicator is associated with a p value of 0.010; and high neutrophil signature, the increase in TWEAK/renal SLEDAI score is associated with a p value 0.0001).

According to some implementations, the system is configured to employ an optimal determination model that includes measures of IFN-Alpha transcript; LDG-associated neutrophil gene signature; and high levels of urinary TWEAK to define a model of independent variables that are additively associated with disease activity. The system can be configured to utilize any combination of biomarkers, for example, highly correlated markers can be used interchangeably in some embodiments (e.g., BAFF and IFN-Alpha, IFN-Alpha and IFN-Gamma, among other options). The system can be configured to determine confidence levels with any combination of measurements available, and report each model resulting score (e.g., SLEDAI) with a determined confidence level for any one or more of multiple measurements of disease activity.

Additional example models employ biomarker combinations of one, two, three, or more of IFN-Alpha, neutrophil gene signature, and TWEAK to score disease activity. Other example models employ any one, two, three, or more biomarkers including IFN-Alpha, neutrophil gene signature, and TWEAK to score at least one component of a disease activity score (e.g., renal SLEDAI).

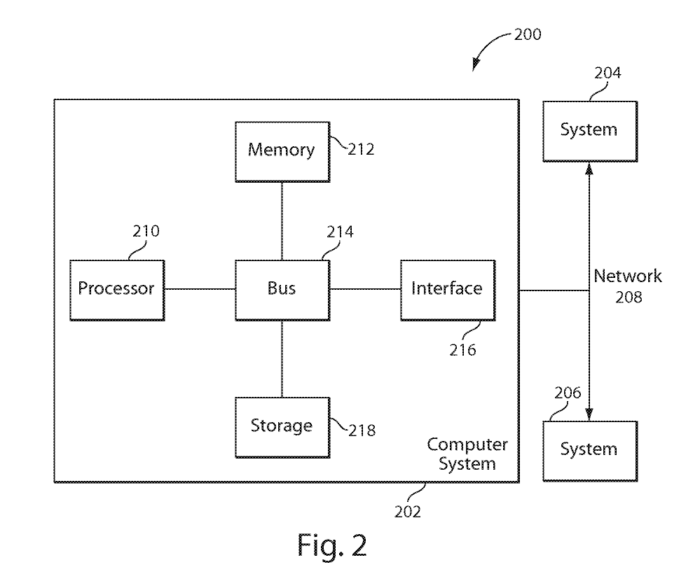

FIG. 2 is a block diagram of a distributed computer system 200, in which various aspects and functions in accord with the present invention may be practiced by specially configuring the computer system. The distributed computer system 200 may include one or more computer systems. For example, as illustrated, the distributed computer system 200 includes three computer systems 202, 204 and 206. As shown, the computer systems 202, 204 and 206 are interconnected by, and may exchange data through, a communication network 208. The network 208 may include any communication network through which computer systems may exchange data. To exchange data via the network 208, the computer systems 202, 204, and 206 and the network 208 may use various methods, protocols and standards including, among others, token ring, Ethernet, Wireless Ethernet, Bluetooth, radio signaling, infra-red signaling, TCP/IP, UDP, HTTP, FTP, SNMP, SMS, MMS, SS2, JSON, XML, REST, SOAP, CORBA HOP, RMI, DCOM and Web Services.

According to some embodiments, the functions and operations discussed for identifying, treating or preventing lupus in a subject can be executed on computer systems 202, 204 and 206 individually and/or in combination. For example, the computer systems 202, 204, and 206 support, for example, participation in collaborative operations, which may include analyzing treatment data captured on a patient population (e.g., the patient having Lupus). In one alternative, a single computer system (e.g., 202) can analyze treatment data captured on a patient population to develop characterization models and/or identify independent indicators for disease activity. The computer systems 202, 204 and 206 may include personal computing devices such as cellular telephones, smart phones, tablets, etc., and may also include desktop computers, laptop computers, etc.

Various aspects and functions in accord with the present invention may be implemented as specialized hardware or software executing in one or more computer systems including the computer system 202 shown in FIG. 2. In one embodiment, computer system 202 is a computing device specially configured to execute the processes and/or operations discussed above. For example, the system can present user interfaces to end-users that present treatment information, diagnostic information, confidence levels associated with biomarkers and/or genetic indicators, among other options. As depicted, the computer system 202 includes at least one processor 210 (e.g., a single core or a multi-core processor), a memory 212, a bus 214, input/output interfaces (e.g., 216) and storage 218. The processor 210, which may include one or more microprocessors or other types of controllers, can perform a series of instructions that manipulate data (e.g., treatment data, testing data, etc.). As shown, the processor 210 is connected to other system components, including a memory 212, by an interconnection element (e.g., the bus 214).

The memory 212 and/or storage 218 may be used for storing programs and data during operation of the computer system 202. For example, the memory 212 may be a relatively high performance, volatile, random access memory such as a dynamic random access memory (DRAM) or static memory (SRAM). In addition, the memory 212 may include any device for storing data, such as a disk drive or other non-volatile storage device, such as flash memory, solid state, or phase-change memory (PCM). In further embodiments, the functions and operations discussed with respect to identifying, treating or preventing lupus in a subject can be embodied in an application that is executed on the computer system 202 from the memory 212 and/or the storage 218.

Computer system 202 also includes one or more interfaces 216 such as input devices, output devices, and combination input/output devices. The interfaces 216 may receive input, provide output, or both. The storage 218 may include a computer-readable and computer-writeable nonvolatile storage medium in which instructions are stored that define a program to be executed by the processor. The storage system 218 also may include information that is recorded, on or in, the medium, and this information may be processed by the application. A medium that can be used with various embodiments may include, for example, optical disk, magnetic disk or flash memory, SSD, among others.

Further, the invention is not limited to a particular memory system or storage system. Although the computer system 202 is shown by way of example as one type of computer system upon which various functions for identifying, treating or preventing lupus in a subject may be practiced, aspects of the invention are not limited to being implemented on the computer system, shown in FIG. 2. Various aspects and functions in accord with the present invention may be practiced on one or more computers having different architectures or components than that shown in FIG. 2.

In some embodiments, the computer system 202 may include an operating system that manages at least a portion of the hardware components (e.g., input/output devices, touch screens, cameras, etc.) included in computer system 202. One or more processors or controllers, such as processor 210, may execute an operating system which may be, among others, a Windows-based operating system (e.g., Windows NT, ME, XP, Vista, 7, 8, or RT) available from the Microsoft Corporation, an operating system available from Apple Computer (e.g., MAC OS, including System X), one of many Linux-based operating system distributions (for example, the Enterprise Linux operating system available from Red Hat Inc.), a Solaris operating system available from Sun Microsystems, or a UNIX operating systems available from various sources. Many other operating systems may be used, including operating systems designed for personal computing devices (e.g., iOS, Android, etc.) and embodiments are not limited to any particular operating system.

According to one embodiment, the processor and operating system together define a computing platform on which applications may be executed. Additionally, various functions for identifying, treating or preventing lupus in a subject may be implemented in a non-programmed environment (for example, documents created in HTML, XML or other format that, when viewed in a window of a browser program, render aspects of a graphical-user interface or perform other functions). Further, various embodiments in accord with aspects of the present invention may be implemented as programmed or non-programmed components, or any combination thereof. Thus, the invention is not limited to a specific programming language and any suitable programming language could also be used.

### EXAMPLES

### Example 1: Identification of Gene Transcripts and Proteins that Independently Predict SLE Disease Activity Over the Next Year

Multiple gene transcripts and proteins in blood or urine have been proposed as biomarkers of disease activity in systemic lupus erythematosus (SLE). However, the relationships between the different biomarkers have rarely been investigated. Understanding which biomarkers independently predict SLE disease activity is important from both a clinical and pathological standpoint. In the present study, the relationship between six transcriptional signatures or proteins is explored and which were independently predictive of future disease activity in subjects with SLE are determined.

### Methods

The SPARE study was a prospective, longitudinal, observational study conducted at a single center. At the initial visit, two proteins and four gene transcripts or signatures were measured in 280 SLE patients. Levels of the BAFF gene transcript, plasma cell gene signature, Type I IFN gene signature, and an LDG-associated Neutrophil gene signature were assessed in PAXgene-preserved peripheral blood by global microarray and qPCR. For proteins, BAFF was measured in serum and TWEAK in urine (both by ELISA). Disease activity during the next year was quantified by SELENA-SLEDAI modified to exclude the immunologic components (complement and dsDNA). Initial exploratory analyses were performed to identify non-linear relationships. Linear regression models were fit to determine which markers were predictive of disease activity in the next year, controlling for age, race, sex, and other markers. Random intercepts were included in the model to account for the correlation between multiple measures from the same patient.

### Results

All markers analyzed except BAFF protein were significantly associated with future disease activity in univariate analyses. The plasma cell gene signature was higher among African Americans, and after controlling for confounding by race, this signature was no longer associated with disease activity. In univariate analysis, the IFN gene signature was highly associated with modified SLEDAI. However, when the associated BAFF mRNA levels were included in the analysis, the IFN signature was no longer significantly associated with disease activity. Table 1 shows the results of a multivariable model. Controlling for sex, race, and other biomarkers, a 1 standard deviation (SD) increase in BAFF was associated with a mean SLEDAI in the follow-up that was 0.26 points higher. The LDG-neutrophil gene signature was not linearly related to disease activity, but for those patients within the top 15% of expression, the average SLEDAI during follow-up was 0.66 points higher than patients with lower levels. Similarly, the relationship between the modified SLEDAI score and urinary TWEAK protein was flat until the 85th percentile of TWEAK after which increasing TWEAK was associated with higher disease activity,

TABLE 1
The estimated degree to which each biomarker
predicts modified SLEDAI in the next year after
adjusting for each other based on a multivariable model.

| | | Effect on Mean | |
|---|---|---|---|
| | | Modified SLEDAI | |
| Marker | Comparison | (95% CI) | P-value |
| BAFF mRNA | Per 1 SD increase | 0.26 (0.09, 0.43) | .0024 |
| Neutrophil Signature | High (top 15%) vs. | 0.66 (0.19, 1.12) | 0.0055 |
| | not high | | |
| Normalized | Per 1 SD above 0.22 | 0.58 (0.25, 0.91) | 0.0006 |
| TWEAK protein | ug TWEAK/mg | | |
| (urine) | creatinine (top 15%) | | |

Note the model also adjusted for sex and race (age was not important).

Conclusion

BAFF gene transcript, LDG-associated neutrophil gene signature, and high levels of urinary TWEAK appear to be independently and additively associated with disease activity. The results presented herein suggest that the association between IFN and disease activity is due to the association between IFN and BAFF, and once BAFF is known, IFN appears unassociated. The results presented herein also suggest that an observed association between PC and disease activity is due to confounding by race. Thus, given that biomarkers are correlated with each other and other risk factors for disease, it is important to adjust for confounding when assessing biomarker/disease relationships.

### Example 2: Identification of Gene Transcripts and Proteins that Independently Predict SLE Disease Activity Over the Next Year

According to various aspects, it is realized that improved identification of biomarkers of SLE disease activity may be needed in order to develop tools for patient management, research, and targets for drug development. For example, multiple gene transcripts and proteins in blood or urine have been found to be correlated with SLE activity. However some of the observed associations might be spurious, due to confounding by correlation with other biomarkers or patient characteristics. The relationships between transcriptional signatures and proteins associated with SLE activity over a 1 year period are analyzed while controlling for potential confounding variables.

### Methods

At the initial visit, two proteins and four gene transcripts or signatures were measured in 280 SLE patients. Levels of the BAFF gene transcript, plasma cell gene signature, Type I IFN gene signature, and an LDG-associated Neutrophil gene signature were assessed in PAXgene-preserved peripheral blood by global microarray and qPCR. For proteins, BAFF was measured in serum and TWEAK in urine (both by ELISA). Disease activity during the next year was quantified by SELENA-SLEDAI modified to exclude the immunologic components (complement and dsDNA).

Repeated measures linear regression models were fit to determine which markers were predictive of disease activity over a one year period, controlling for age, race, sex, and other markers. Non-linear relationships between biomarker levels and disease activity were also explored.

### Results

In univariate analyses, all markers analyzed except BAFF protein were significantly associated with future disease activity. After controlling for race, the PC signature was no longer significantly associated. After controlling for BAFF mRNA levels, the IFN signature was no longer significantly associated. Controlling for sex, race, and other biomarkers it was found that: 1) a 1 standard deviation (SD) increase in BAFF was associated with a mean SLEDAI increase of 0.26 in the follow-up (p=0.0034), 2) those patients within the top 15% of the Neutrophil gene signature expression had a 0.66 higher mean SLEDAI during follow-up (p=0.0056), and 3) the relationship between the SLEDAI score and urinary TWEAK protein was constant until the 85th percentile of TWEAK after which a 1 SD increase in TWEAK was associated with a 0.58 increase in mean SLEDAI (p=0.0006). In a similar analysis focusing on renal disease activity, a 1 SD increase in the IFN signature (rather than BAFF) was associated with a mean renal SLEDAI increase of 0.11 in the follow-up period (p=0.04), The Neutrophil signature remained significant at a similar level as for overall disease activity, and the relationship between renal SLEDAI score and urinary TWEAK was linear with a 1 SD increase in TWEAK being associated with a 0.25 increase in mean renal SLEDAI (p<0.0001).

### Example 3: Identification of Gene Transcripts and Proteins that Independently Predict SLE Disease Activity Over the Next Year

Various data models can be used to evaluate patient data. According to one embodiment, a normalized UTWEAK data set is evaluated along with the other variables.

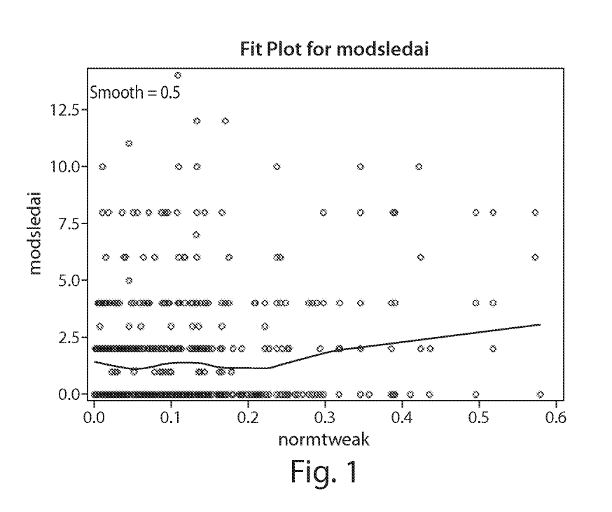

The main findings are as follows:
The relationship between normalized UTWEAK and a modified SLEDAI score appears flat until the upper 15 percent of the UTWEAK values. After that point, the larger the TWEAK, the higher the average disease activity.
BAFF protein is not predictive, PC is not predictive after controlling for race, an IFN-Gamma is not predictive after controlling for BAFF, and that Neutrophils appears important as an indicator after a certain threshold value.
The interaction between UTWEAK and Neutrophils is not as strong (p=0.02) in un-normalized models. For the purposes of clarity this relationship is not included in the modelled data show in FIG. 1.
There is also some evidence of an interaction between UTWEAK and BAFF, but again it was not strong (p=0.02). For the purposes of clarity this relationship is not includes in the modelled data show in Fig. below
Three biomarkers (BAFF, Neutrophil, and UTWEAK) are all independent predictors of modified SLEDAI in the next year

FIG. 1 illustrates the relationship between normalized UTWEAK and modified SLEDAI. It shows a smooth estimate of the mean modified SLEDAI as a function of Normalized UTWEAK

TABLE 2
Model showing the degree to which each biomarker predicts
modified SLEDAI in the next year.¹

| | | Effect on Mean | |
|---|---|---|---|
| | | Modified SLEDAI | |
| Marker | Comparison | (95% CI) | P-value |
| BAFF Signature | Per 1 SD increase | 0.26 (0.09, 0.43) | .0024 |
| Neutrophil | High (top 15%) vs. | 0.66 (0.19, 1.12) | 0.0055 |
| Signature | not high | | |
| Normalized | Per 1 SD above 0.22 | 0.58 (0.25, 0.91) | 0.0006 |
| UTWEAK | | | |

¹Note the model also controlled for sex and race (age was not important).

### Example 4: Identification of Gene Transcripts and Proteins that Independently Predict SLE Disease Activity Over the Next Year

Multiple gene transcripts and proteins in blood or urine have been proposed as biomarkers of disease activity in systemic lupus erythematosus (SLE). Understanding which biomarkers independently predict SLE disease activity is important from both a clinical and pathological standpoint. In the present study, the relationship between seven transcriptional signatures or proteins was explored over a 1 year period.

### Methods

The SPARE study was a prospective, longitudinal, observational study conducted at a single center. At the initial visit, two proteins and four gene transcripts or signatures were measured in 286 SLE patients. Characteristics of the patients are shown in Table 3. Levels of the BAFF gene transcript, plasma cell gene signature, IFN-alpha gene signature, IFN-gamma gene signature, and LDG-associated neutrophil gene signature were assessed in PAXgene-preserved peripheral blood by global microarray and qPCR. TWEAK protein was measured in urine by ELISA. Disease activity during the next year was quantified quarterly by SELENA-SLEDAI modified to exclude the immunologic components (complement and dsDNA—“ModSLEDAI”). Non-parametric regression (Loess) was used to explore the shape of relationship between biomarkers and mean SLE activity. Linear regression models were fit to determine which markers were predictive of disease activity over a one year period, controlling for age, race, sex, and other markers.

TABLE 3
Characteristics of 286 Patients

| Characteristic | Number (%) | |
|---|---|---|
| Female | 260 | (91%) |
| Race | | |
| Caucasian | 170 | (49%) |
| Af. American | 95 | (33%) |
| Other | 21 | (7%) |
| Age | | |
| >30 | 27 | (9%) |
| 30-44 | 97 | (34%) |
| 45-59 | 119 | (42%) |
| 60+ | 43 | (15%) |
| Time since SLE Dx | | |
| <1 year | 13 | (5%) |
| 1-4 years | 56 | (21%) |
| 4+years | 202 | (75%) |

### Results

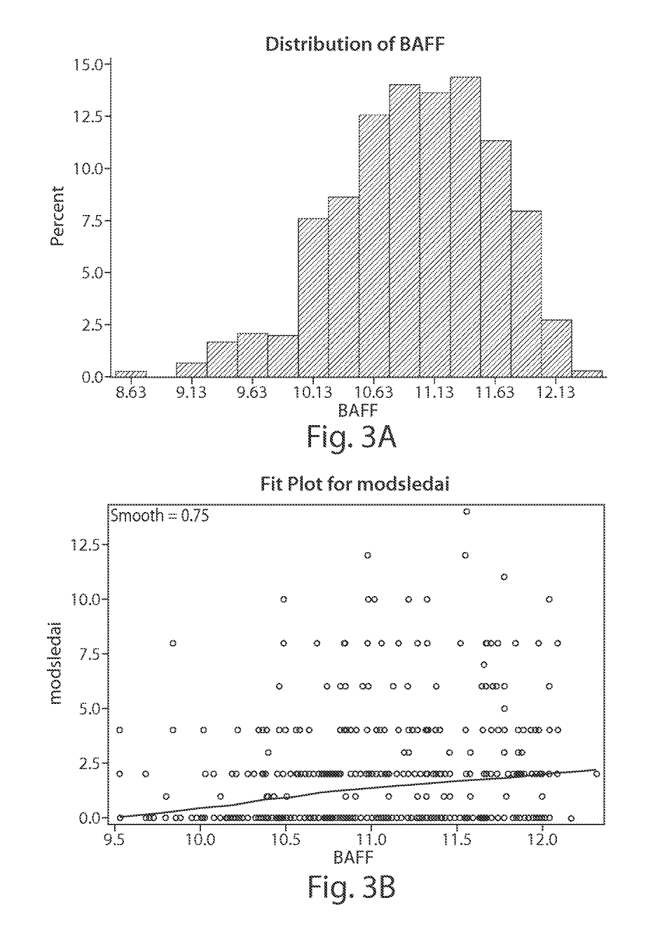

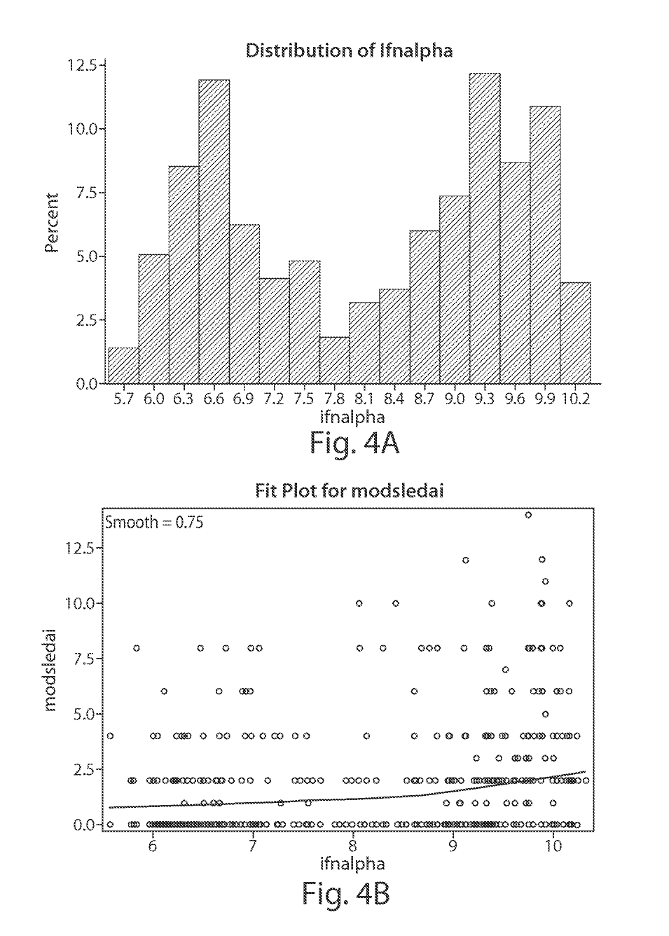

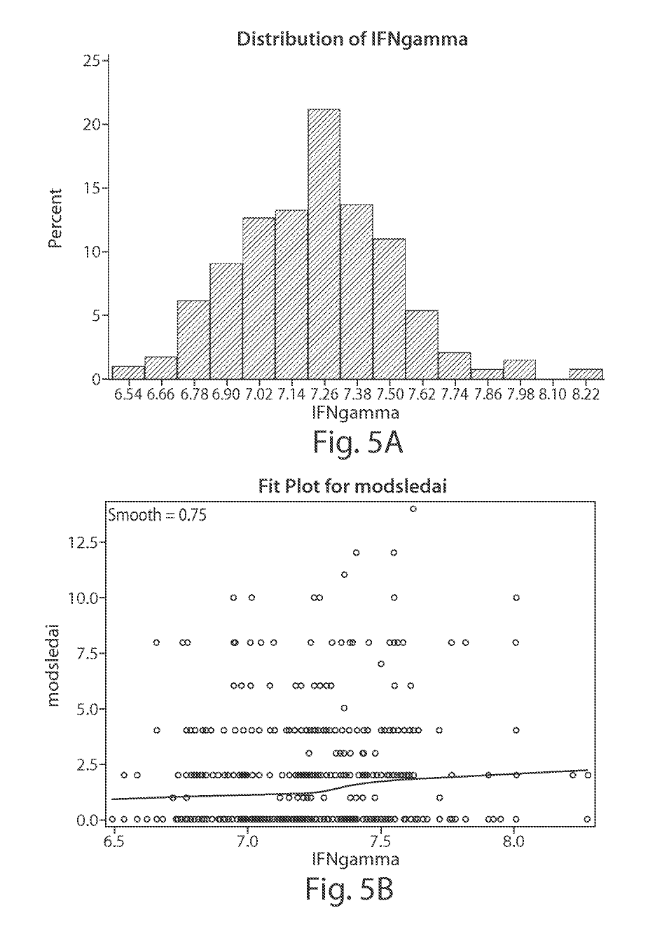

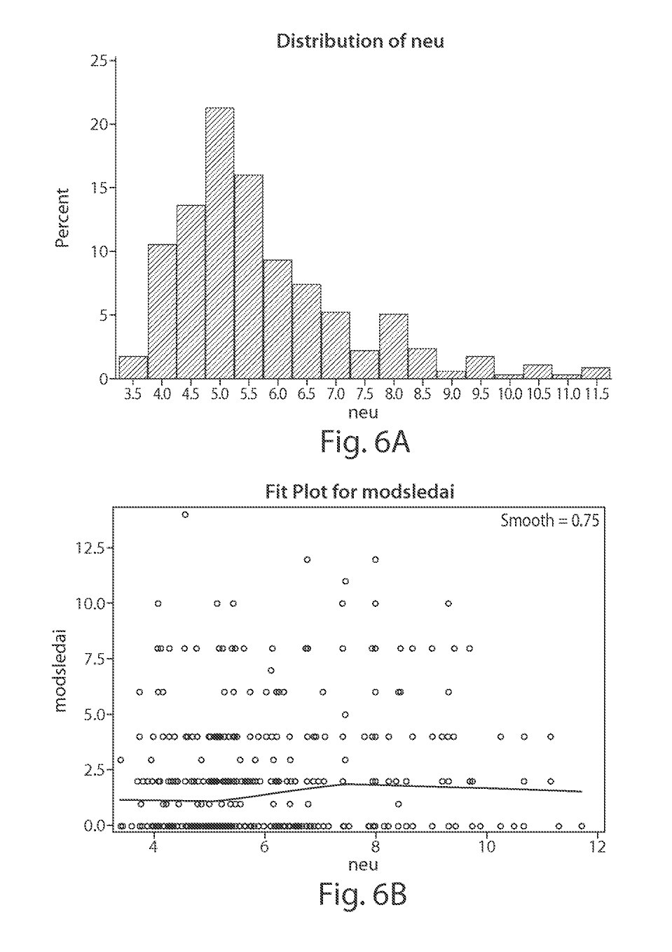

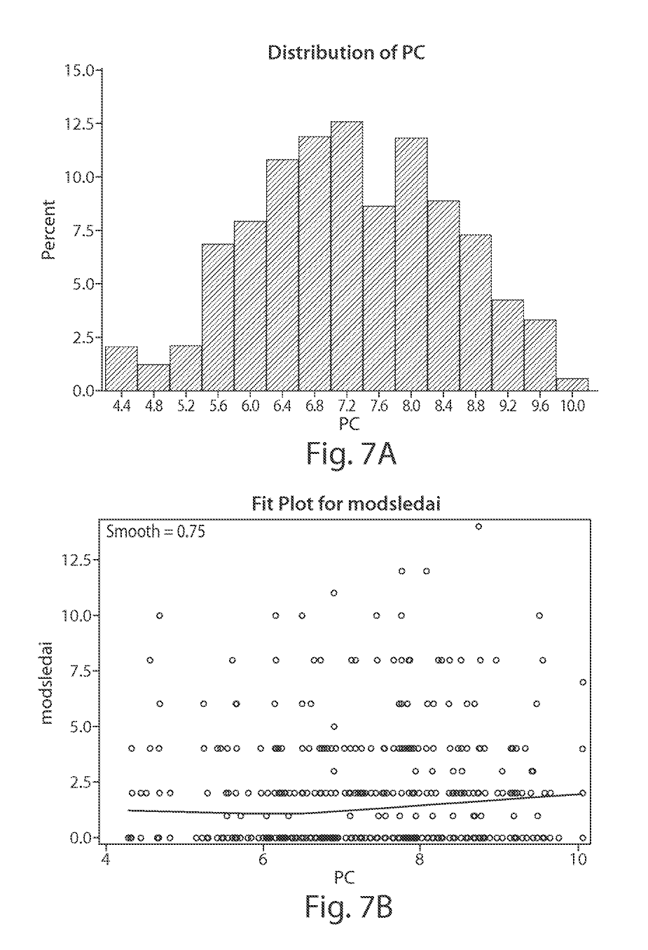

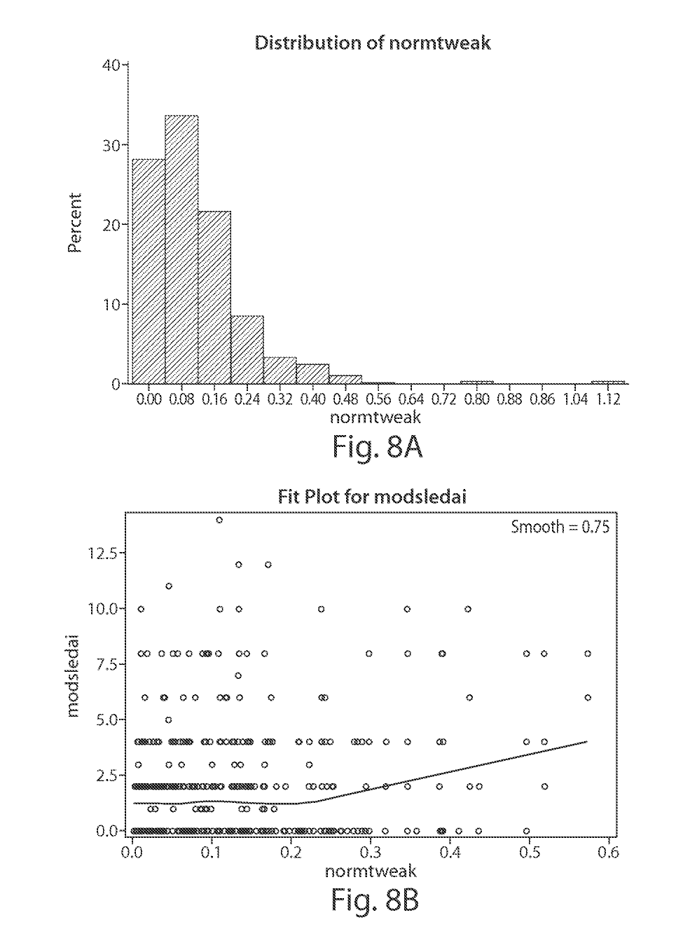

The correlation of each of the biomarkers with SLE activity was examined by univariate analysis. For each biomarker, the distribution of levels within the patient population and the correlation between the biomarker and ModSLEDAI level was determined. For BAFF gene transcript expression, p<0.0001 for linear increase in mean ModSLEDAI (FIG. 3A and FIG. 3B). The IFN-alpha gene signature was calculated based on the geometric mean of the expression levels of 8 IFN-induced genes, including IFIT1; MX2; OAS1; OAS2; OASL; HERCS; HES4; and RSAD2, as defined by Hall, J C et al. (PNAS 2012). For the IFN-alpha transcript, p<0.0001 for linear increase in mean ModSLEDAI for values above 8.0 (FIG. 4A and FIG. 4B). The IFN-gamma gene signature was calculated based on expression of a set of IFN-gamma induced genes, including AIM2; CD74; CXCL10; CXCL9; GBP2; GBP4; GBP5; HLA-DMA; HLA-DRA; HLA-DRB4; HLA-DRB6; IL18BP; INDO/IDO1; IRF1; LOC400759; PSMB10; RARRES3; SERPINB2; TNFRSF14; and WARS, as defined by Hall, J C et al. (PNAS 2012). For the IFN-gamma transcript, p=0.011 for higher mean ModSLEDAI for values above 7.25 (FIG. 5A and FIG. 5B). The LDG-enriched neutrophil gene signature was calculated as the geometric mean of the expression levels of 8 genes upregluated in LDG (BPI, CEACAM6, CEACAM8, DEFA4, LCN2, LTF, MMP8, and OLFM4). For the LDG-enriched neutrophil gene signature, p=0.0040 for higher mean ModSLEDAI for values above 7.0 (FIG. 6A and FIG. 6B). The plasma cell gene signature was calculated based on the geometric mean of the expression of two genes encoding immunoglobulin J chain (IGJ) and thioredoxin domain-containing protein 5 (TXNDCS), which are known to be specific to plasma cells. For the plasma cell gene signature, p=0.0039 for linear increase in mean ModSLEDAI for values above 7.0 (FIG. 7A and FIG. 7B). For TWEAK, p=0.0002 for increase for values above 0.22 (FIG. 8A and FIG. 8B).

The correlation between markers was also evaluated. BAFF and IFN-alpha were very highly correlated (correlation coefficient=0.78). IFN-alpha and IFN-gamma were highly correlated (correlation coefficient 0.60). The plasma cell signature had moderate correlation with BAFF and IFN-alpha and IFN-gamma (correlation coefficients 0.22-0.23). The neutrophil gene signature and TWEAK were not strongly correlated with other markers (correlation coefficients <0.15).

Table 4 shows the results of a multivariable model for disease activity measured using ModSLEDAI. Controlling for sex, race, and other biomarkers, IFN-alpha gene signature, LDG-associated neutrophil gene signature, and high levels of urinary TWEAK were additively associated with disease activity. For those in the top 55% of IFN-alpha, a 1 standard deviation increase in IFN-alpha was associated with 0.61 points higher mean ModSLEDAI (p=0.0002). The top 15% of the neutrophil gene signature expression was associated with 0.62 points higher ModSLEDAI (p=0.0090). For those in the top 15% of TWEAK, a 1 SD increase in TWEAK associated with 0.57 points higher ModSLEDAI (p=0.0006).

TABLE 4
Multiple variable model for disease activity (ModSLEDAI)
controlling for sex, race and other biomarkers.

| | | p-value |
|---|---|---|
| IFN-alpha | For those in the top 55% of | 0.0002 |
| | IFN-alpha, a 1 SD increase | |
| | in IFN-alpha was associated | |
| | with 0.61 points higher | |
| | mean SLEDAI | |
| Neutrophil Gene Signature | Top 15% of the Neutrophil | 0.0090 |
| | gene signature expression | |
| | associated with 0.62 points | |
| | higher SLEDAI | |
| Urinary TWEAK protein | For those in top 15% of | 0.0006 |
| | TWEAK, a 1 SD increase in | |
| | TWEAK associated with | |
| | 0.57 points higher SLEDAI | |

Table 5 shows the results of a multivariable model for renal disease activity measured using renal SLEDAI. Controlling for sex, race, and other biomarkers, IFN-alpha gene signature, LDG-associated neutrophil gene signature, and high levels of urinary TWEAK were additively associated with disease activity For those in the top 50% of IFN-alpha, a 1 standard deviation increase in IFN-alpha was associated with 0.33 points higher mean renal component of SLEDAI (p=0.024). On average, high neutrophil signature is associated with 0.39 higher mean renal component of SLEDAI (p=0.010). On average, high neutrophil signature is associated with 0.39 higher mean renal component of SLEDAI (p=0.0001).

TABLE 5
Multiple variable model for renal disease activity
(renal SLEDAI) controlling for sex, race and other biomarkers.

| | | p-value |
|---|---|---|
| IFN-alpha | For those in the top 50% of | 0.024 |
| | IFN-alpha, a 1 SD increase | |
| | in IFN-alpha was associated | |
| | with 0.33 points higher | |
| | mean renal component of | |
| | SLEDAI | |
| Neutrophil Gene Signature | On average, high neutrophil | 0.010 |
| | signature is associated with | |
| | 0.39 higher mean renal | |
| | component of SLEDAI | |
| Urinary TWEAK protein | For those with high | 0.0001 |
| | neutrophil signature, 1 SD | |
| | increase in TWEAK | |
| | associated with 0.57 | |
| | increase in mean renal | |
| | component of SLEDAI | |

### Conclusions

The results presented herein suggest IFN-alpha gene signature, LDG-associated neutrophil gene signature, and high levels of urinary TWEAK were independently and additively associated with disease activity. After controlling for race, the plasma cell gene signature was no longer significantly associated with ModSLEDAI. After controlling for IFN-alpha gene signature, neither BAFF gene expression nor IFN-gamma were significantly associated with ModSLEDAI. Given that biomarkers are correlated with each other and other risk factors for disease, it is important to adjust for confounding effects when assessing biomarker/disease relationships.

### INCORPORATION BY REFERENCE

The contents of all references, figures, sequence listing, patents and published patent applications cited throughout this application are hereby incorporated by reference. All publications, patents, and patent applications mentioned herein are hereby incorporated by reference in their entirety as if each individual publication, patent or patent application was specifically and individually indicated to be incorporated by reference.

Also incorporated by reference in their entirety are any polynucleotide and polypeptide sequences which reference an accession number correlating to an entry in a public database, such as those maintained by The Institute for Genomic Research (TIGR) on the worldwide web at tigr.org and/or the National Center for Biotechnology Information (NCBI) on the worldwide web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed.

What is claimed is:

1. A method of evaluating a subject having lupus, comprising:
acquiring a value of disease progression (e.g., from a sample obtained from the subject), wherein the value of disease progression comprises a measure of two, three or all four of: a TWEAK biomarker (e.g., urinary TWEAK (UTWEAK)), a neutrophil gene signature, a BAFF biomarker (e.g., BAFF mRNA), or an interferon (IFN) signature,
thereby evaluating the subject.

2. A method of evaluating or monitoring the effectiveness of a lupus therapy in a subject having lupus comprising:
acquiring a value of disease progression (e.g., from a sample obtained from the subject), wherein the value of disease progression comprises a measure of two, three or all four of: a TWEAK biomarker (e.g., urinary TWEAK (UTWEAK)), a neutrophil gene signature, a BAFF biomarker (e.g., BAFF mRNA), or an interferon (IFN) signature,
thereby evaluating or monitoring the effectiveness of the lupus therapy in the subject.

3. A lupus therapy for use in a method of treating or preventing lupus in a subject, comprising:
acquiring a value of disease progression that comprises a measure of two, three or all four of: a TWEAK biomarker (e.g., urinary TWEAK (UTWEAK)), a neutrophil gene signature, a BAFF biomarker (e.g., BAFF mRNA), or an interferon (IFN) signature, and
responsive to a determination of the value of disease progression:
administering a lupus therapy (e.g., a first or a second lupus therapy);
selecting or altering a dosing of a lupus therapy (e.g., a first or a second lupus therapy); or
selecting or altering the schedule or time course of a lupus therapy (e.g., a first or a second lupus therapy);
wherein the lupus therapy comprises one or more of: a nonsteroidal anti-inflammatory drug (NSAID); an antimalarial drug (e.g., hydroxychloroquine); a corticosteroid (e.g., a glucocorticoid); an immunosuppressant (e.g., azathioprine, mycophenolate mofetil, or methotrexate); an intravenous immunoglobulin; an anti-TWEAK antibody; an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; or an anti-BAFF antibody.

4. A method of treating or preventing lupus in a subject, comprising:
acquiring a value of disease progression that comprises a measure of two, three or all four of: a TWEAK biomarker (e.g., urinary TWEAK (UTWEAK)), a neutrophil gene signature, a BAFF biomarker (e.g., BAFF mRNA), or an interferon (IFN) signature, and
responsive to a determination of the value of disease progression, performing one, two, three, four or more of:
administering a lupus therapy (e.g., a first or a second lupus therapy);
identifying the subject as disease progressing;
selecting a first therapy or an alternative (e.g., a second) lupus therapy;
selecting or altering a dosing of a lupus therapy (e.g., a first or a second lupus therapy); or
selecting or altering the schedule or time course of a lupus therapy (e.g., a first or a second lupus therapy),
thereby treating or preventing lupus in the subject.

5. The method or use of any of claims 1-4, which further comprises one or more of the following:
(i) identifying the subject as being in need of a lupus therapy (e.g., a first lupus therapy, or an alternative (e.g., second) lupus therapy);
(ii) identifying the subject as having an increased or a decreased response to a lupus therapy (e.g., a first lupus therapy, or a second lupus therapy);
(iii) identifying the subject as being stable or showing an improvement in one or more abilities or function, or showing a decline in one or more abilities or function;
(iv) diagnosing and/or prognosing the subject;
(v) selecting or altering the course of, a lupus therapy (e.g., a first lupus therapy, a dose, a treatment schedule or time course, and/or the use of an alternative (e.g., second) lupus therapy);
(vi) determining lupus disease progression in the subject;
(vii) administering a lupus therapy (e.g., a first lupus therapy, or an alternative (e.g., second) lupus therapy to the subject); and/or
(viii) evaluating the effectiveness of a therapy (e.g., a lupus therapy) in treating or preventing a progressive form of lupus,
wherein a change in the disease progression value relative to a specified or reference value indicates one or more of: identifies the subject as being in need of the first lupus therapy, or the alternative or second lupus therapy; identifies the subject as having an increased or decreased response to the first or second therapy; determines the treatment to be used; and/or determines or predicts the time course of the onset and/or progression of lupus.

6. The method or use of any of claims 3-5, wherein the lupus therapy comprises one or more of: a nonsteroidal anti-inflammatory drug (NSAID); an antimalarial drug (e.g., hydroxychloroquine); a corticosteroid (e.g., a glucocorticoid); an immunosuppressant (e.g., azathioprine, mycophenolate mofetil, or methotrexate); an intravenous immunoglobulin; an anti-TWEAK antibody; an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; or an anti-BAFF antibody.

7. The method or use of any of claims 3-6, wherein the lupus therapy comprises a first therapy chosen from one or more of:
(i) nonsteroidal anti-inflammatory drug (NSAID);
(ii) an antimalarial drug (e.g., hydroxychloroquine);
(iii) a corticosteroid,
(iv) an immunosuppressant chosen from azathioprine, mycophenolate mofetil, or methotrexate; or
(v) an intravenous immunoglobulin.

8. The method or use of any of claims 1-7, wherein the lupus therapy comprises a second therapy chosen from one or more of:
(i) an anti-TWEAK antibody;
(ii) an anti-CD40L antibody;
(iii) an anti-CD40 antibody;
(iv) an anti-CD20 antibody;
(v) an anti-interferon antibody;
(vi) rapamycin;
(vii) arsenic trioxide;
(viii) 5-chloro-N-ethyl-4-hydroxy-1-methyl-2-oxo-N-phenyl-1,2-dihydroquinoline-3-carboxamide;
(ix) an anti-BDCA2 antibody; or
(x) an anti-BAFF antibody.

9. The method or use of any of claims 1-8, wherein the value of disease progression comprises a measure of a combination of a gene signature and a biomarker.

10. The method or use of any of claims 1-9, wherein the value of disease progression comprises a measure of a combination of two of: a BAFF biomarker (e.g., BAFF mRNA), a TWEAK biomarker (e.g., UTWEAK), or a neutrophil gene signature.

11. The method or use of any of claims 1-9, wherein the value of disease progression comprises a measure of a combination of all three of: a BAFF biomarker (e.g., BAFF mRNA), a TWEAK biomarker (e.g., UTWEAK), and a neutrophil gene signature.

12. The method or use of any of claims 1-9, wherein the value of disease progression comprises a measure of a neutrophil gene signature and one or both of: a BAFF biomarker (e.g., BAFF mRNA) or a TWEAK biomarker (e.g., UTWEAK).

13. The method or use of any of claims 1-9, wherein the value of disease progression comprises a measure of a combination of two or all of: an interferon signature (e.g., IFN-alpha signature), a neutrophil gene signature, or a TWEAK biomarker (e.g., urinary TWEAK or UTWEAK).

14. The method or use of any of claims 1-9, wherein the value of disease progression comprises a measure of a neutrophil gene signature, and one or both of an interferon signature (e.g., IFN-alpha signature) or a TWEAK biomarker (e.g., urinary TWEAK or UTWEAK).

15. The method or use of any of claims 1-9, wherein the value of disease progression comprises a measure of an interferon signature (e.g., IFN-alpha signature), and one or both of a neutrophil gene signature or a TWEAK biomarker (e.g., urinary TWEAK or UTWEAK).

16. The method or use of any of claims 1-9, wherein value of disease progression comprises a measure of a TWEAK biomarker (e.g., urinary TWEAK or UTWEAK), and one or both of an interferon signature (e.g., IFN-alpha signature) or a neutrophil gene signature.

17. The method or use of any of claim 1-9 or 13-16, wherein the value of disease progression comprises a measure of an IFN signature as an indication of renal disease activity.

18. The method or use of any of claims 1-17, wherein the value of disease progression is associated with a multivariate model.

19. The method or use of any of claims 1-18, further comprising determining a confidence level associated with the value of disease progression.

20. The method or use of claim 19, wherein the confidence level is increased as the number of measures of biomarker and signature increases.

21. The method or use of any of claims 1-20, wherein an increase in the measure of the BAFF biomarker of about 1 standard deviation (SD) relative to reference value (e.g., a median value for a lupus patient population) is indicative of a mean average increase of from about 0.05 to about 0.45 of the SLEDAI score (e.g., mean SLEDAI score).

22. The method or use of any of claims 1-21, wherein an increase in the measure of the BAFF biomarker of about 1 standard deviation (SD) relative to reference value (e.g., a median value for a lupus patient population) is indicative of an increase in a range of from 0.09 to 0.40, 0.15 to 0.35, 0.2 to 0.3, or 0.25-0.26 of the SLEDAI score (e.g., mean SLEDAI score).

23. The method or use of any of claims 1-22, wherein the measure of the TWEAK (e.g., urinary TWEAK) biomarker being in the top 15% of measures of the TWEAK biomarker in a lupus patient population is indicative of a mean average increase of from about 0.20 to about 0.95 of the SLEDAI score (e.g., mean SLEDAI score).

24. The method or use of any of claims 1-23, wherein the measure of the TWEAK (e.g., urinary TWEAK) biomarker being in the top 15% of measures of the TWEAK biomarker in a lupus patient population is indicative of an increase in a range of from 0.25 to 0.90, 0.3 to 0.8, 0.4 to 0.7, or 0.5-0.6 of the SLEDAI score (e.g., mean SLEDAI score).

25. The method or use of any of claims 1-24, wherein the measure of the neutrophil gene signature being in the top 15% of measures of the neutrophil gene signature in a lupus patient population is indicative of a mean average increase of from about 0.15 to about 1.5 of the SLEDAI score (e.g., mean SLEDAI score).

26. The method or use of any of claims 1-25, wherein the measure of the neutrophil gene signature being in the top 15% of measures of the neutrophil gene signature is in the top 15% of a lupus patient population is indicative of an increase in a range of from 0.2 to 1.15, 0.3 to 1, 0.4 to 0.9, 0.5-0.8, or 0.6 to 0.7 of the SLEDAI score (e.g., mean SLEDAI score).

27. The method or use of any of claims 1-26, wherein an increase in the measure of the IFN signature of about 1 standard deviation (SD) relative to reference value (e.g., a median value for a lupus patient population) is indicative of an increase in a range of from about 0.01 to about 0.30, 0.05 to 0.20, 0.08 to 0.15, 0.1 to 0.12, or about 0.11 of the renal SLEDAI score (e.g., mean renal SLEDAI score).

28. The method or use of any of claims 1-27, wherein an increase in the measure of the neutrophil signature of about 1 standard deviation (SD) relative to reference value (e.g., a median value for a lupus patient population) in the measure of the TWEAK biomarker (e.g., urinary TWEAK) is indicative of an increase in a range of from about 0.10 to about 0.35, 0.15 to 0.3, 0.20 to 0.26, or about 0.25 of the renal SLEDAI score (e.g., mean renal SLEDAI score).

29. The method or use of any of claims 1-28, wherein an increase in the measure of the neutrophil gene signature is indicative of a mean average increase of from about 0.1 to about 1, 0.2 to 0.8, 0.3 to 0.5, 0.3 to 0.4, or 0.39 of the renal SLEDAI score (e.g., a mean renal SLEDAI score).

30. The method or use of any of claims 1-29, wherein an increase in the measure of the IFN signature (e.g., IFN-alpha signature) being in the top 55% in a lupus patient population (e.g., a median value for a lupus patient population) of about 1 standard deviation (SD) relative to reference value (e.g., a median value for a lupus patient population) is indicative of an increase in a range of from about 0.2 to 1.15, 0.3 to 1, 0.4 to 0.9, 0.5-0.8, 0.6 to 0.7, or about 0.61 of the SLEDAI score (e.g., mean SLEDAI score).

31. The method or use of any of claims 1-30, wherein an increase in the measure of the IFN signature (e.g., IFN-alpha signature) being in the top 50% in a lupus patient population (e.g., a median value for a lupus patient population) of about 1 standard deviation (SD) relative to reference value (e.g., a median value for a lupus patient population) is indicative of an increase in a range of from about 0.1 to 1, 0.2 to 0.8, 0.3 to 0.6, 0.3-0.4, or about 0.33 of the renal SLEDAI score (e.g., mean renal SLEDAI score).

32. The method or use of any of claims 1-31, wherein an increase in the measure of the TWEAK biomarker of about 1 standard deviation (SD) relative to reference value (e.g., a median value for a lupus patient population) in the measure of the TWEAK biomarker (e.g., urinary TWEAK) is indicative of an increase in a range of from about 0.1 to about 1, 0.3 to 0.7, 0.4 to 0.6, or about 0.57 of the renal SLEDAI score (e.g., mean renal SLEDAI score) in subjects having a high level of a neutrophil signature.

33. The method or use of any of claims 1-32, wherein the measure of biomarker and/or signature is acquired before, at the same time, or during the course of a lupus therapy.

34. The method or use of any of claims 1-33, wherein the measure of biomarker and/or signature is acquired at least one, two, three, four, five, six months, 1 or 2 years, or longer after the initiation of a lupus therapy.

35. The method or use of any of claims 21-34, wherein the SLEDAI score is acquired at least three, four, five, or six months or 1 or 2 years after the value of disease progression is acquired.

36. The method or use of any of claims 21-34, wherein the SLEDAI score is acquired at least 1 or 2 years after the value of disease progression is acquired.

37. The method or use of any of claims 1-36, wherein the BAFF biomarker comprises a value for expression of the gene.

38. The method or use of any of claims 1-37, wherein the neutrophil gene signature comprises a value for expression of at least 5, 6, 7, 8, 9 or 10 genes comprising a neutrophil gene signature.

39. The method or use of any of claims 1-38, wherein the value for expression of the biomarker or gene signature comprises a value for a transcriptional parameter, e.g., the level of an mRNA encoded by the gene.

40. The method or use of any of claims 1-38, wherein value for expression of the biomarker comprises a value for a translational parameter, e.g., the level of a soluble protein.

41. The method or use of any of claims 1-40, wherein the TWEAK biomarker (e.g., urinary TWEAK) comprises a value for expression of the protein, e.g., a TWEAK soluble protein.

42. The method or use of any of claims 1-41, further comprising obtaining a sample from the subject, wherein the sample is chosen from a non-cellular body fluid; or a cellular or tissue fraction.

43. The method or use of claim 42, wherein the non-cellular body fluid is urine.

44. The method or use of claim 42, wherein the cellular fraction comprises peripheral blood.

45. The method or use of any of claim 5, 21-22, 27-28 or 30-32, wherein the reference value is obtained from one or more of: a baseline or prior value for the subject, the subject at a different time interval, an average or median value for a lupus patient population, a healthy control, or a healthy subject population.

46. The method or use of any of claims 1-45, wherein the disease progression in the lupus subject comprises a steady worsening of symptoms over time.

47. The method or use of claim 46, wherein symptoms of lupus comprise seizure, psychosis, organic brain syndrome, visual disturbance, cranial nerve disorder, lupus headache, cerebrovascular accident, vasculitis, arthritis, myositis, urinary casts, hematuria, proteinuria, pyuria, rash, alopecia, mucosal ulcers, pleurisy, pericarditis, low complement, increased DNA binding, fever, thrombocytopenia, and/or leukopenia.

48. The method or use of any of claims 1-47, wherein a SLEDAI score of between 1-5 is indicative of mild disease activity in the subject; a SLEDAI score of between 6-10 is indicative of moderate disease activity in the subject; a SLEDAI score of between 11-19 is indicative of high disease activity in the subject; a SLEDAI score of 20-105 is indicative of very high disease activity in the subject.

49. The method or use of any of claims 1-48, wherein said method further comprises treating, or preventing in, the subject having lupus one or more symptoms associated with lupus by administering to a subject a lupus therapy, in an amount sufficient to reduce one or more symptoms associated with lupus.

50. The method or use of claim 49, wherein said treating or preventing comprises reducing, retarding or preventing, a flare, or the worsening of the disease, in the lupus subject.

51. The method or use of any of claims 1-50, wherein a second or an alternative therapy is administered when a patient is less responsive or shows disease progression when treated with the first therapy.

52. The method or use of any of claims 1-51, wherein lupus comprises systemic lupus erythematosus (SLE) (e.g., lupus nephritis), cutaneous lupus erythematosus (CLE) (e.g., acute cutaneous lupus erythematosus (ACLE), subacute cutaneous lupus erythematosus (SCLE), intermittent cutaneous lupus erythematosus, and chronic cutaneous lupus), drug-induced lupus, and neonatal lupus.

53. The method or use of any of claims 1-52, lupus comprises SLE.

54. The method or use of any of claim 21-32, 35-36, or 48, wherein the SLEDAI scale comprises the SELENA SLEDAI scale.

55. The method or use of any of claims 1-54, wherein the neutrophil gene signature comprises a low density granulocyte (LDG) neutrophil gene signature.

56. A kit for evaluating a lupus patient, comprising:
a means or tests for evaluating two, three or all four of: a TWEAK biomarker (e.g., urinary TWEAK (UTWEAK)), a neutrophil gene signature, a BAFF biomarker (e.g., BAFF mRNA), or an interferon (IFN) signature; and
a means for determining a value of disease progression associated with the subject, prior to, during, and/or after a lupus therapy.

57. The kit of claim 56, further comprising means for evaluating the SLEDAI scale.

58. A system for evaluating lupus in a subject, comprising:
at least one processor operatively connected to a memory, the at least one processor when executing is configured to perform any one of the steps recited in claims 1-55.

59. A system for evaluating lupus in a subject, comprising:
at least one processor operatively connected to a memory, the at least one processor when executing is configured to:
acquire a value of disease progression that comprises a measure of two, three or all four of: a TWEAK biomarker (e.g., urinary TWEAK (UTWEAK)), a neutrophil gene signature, a BAFF biomarker (e.g., BAFF mRNA), or an interferon (IFN) signature, to score disease activity, and
responsive to a determination of the value of disease progression, perform one, two, three, four or more of:
identify the subject as disease progressing;
recommend a lupus therapy; or
recommend a selection or alteration of:
(i) a dosing of a lupus therapy;
(ii) a schedule or time course of a lupus therapy; or
(iii) a second lupus therapy.

60. A system for evaluating lupus in a subject, comprising:
at least one processor operatively connected to a memory, the at least one processor when executing is configured to
accept patient test information on one or more indicators for lupus disease activity;
evaluate at least one, two, three, or all, independent indicators of disease progression chosen from a TWEAK biomarker (e.g., urinary TWEAK (UTWEAK)), a neutrophil gene signature, a BAFF biomarker (e.g., BAFF mRNA), or an interferon (IFN) signature;
determine a confidence level associated with a prognoses of disease progression based on a multivariate model applied to the at least one, two, or all, independent indicators of disease progression; and
display the confidence level associated with the prognoses of disease progression.

61. A system for evaluating lupus in a subject, comprising:
at least one processor operatively connected to a memory, the at least one processor when executing is configured to
accept patient test information on one or more indicators for lupus disease activity;
evaluate at least one, two, three, or all, indicators of disease progression
identify independent indicators of disease progression from the at least one, two, three, or all of indicators of disease progression chosen from a TWEAK biomarker (e.g., urinary TWEAK (UTWEAK)), a neutrophil gene signature, a BAFF biomarker (e.g., BAFF mRNA), or an interferon (IFN) signature;
determine a confidence level associated with a prognoses of disease progression based on a multivariate model applied to the at least one, two, three, or all, available indicators of disease progression; and
display the confidence level associated with the prognoses of disease progression.

62. The system according to claim 61, wherein the at least one processor is further configured to select indicators of disease progression to generate the multivariate model based on identification of independent indicators of disease progression.

63. The system according to any of claims 61-62, wherein the at least one processor when executing is further configured to select the multivariate model to apply to the at least one, two, three, or all, available indicators of disease progression responsive to the confidence level associated with the identified independent indicators of disease progression.

64. The system according to any of claims 61-63, wherein the at least one processor is further configured to optimize the determination of the prognoses of disease progression responsive to maximizing the confidence level associated with the independent indicators of disease progression.

65. The system according to any of claims 61-64, wherein the at least one processor is further configured to optimize the determination of the prognoses of disease progression responsive to substituting highly correlated indicators chosen from BAFF and IFN-Alpha, IFN-Alpha and IFN-Gamma, where the corresponding independent indicator is unavailable or suspect.

66. The system according to claim 59, wherein the lupus therapy comprises one or more of: a nonsteroidal anti-inflammatory drug (NSAID); an antimalarial drug (e.g., hydroxychloroquine); a corticosteroid (e.g., a glucocorticoid); an immunosuppressant (e.g., azathioprine, mycophenolate mofetil, or methotrexate); an intravenous immunoglobulin; an anti-TWEAK antibody; an anti-CD40L antibody; an anti-CD40 antibody; an anti-CD20 antibody; an anti-interferon antibody; rapamycin; arsenic trioxide; 5-chloro-N-ethyl-4-hydroxy-1-methyl-2-oxo-N-phenyl-1,2-dihydroquinoline-3-carboxamide; an anti-BDCA2 antibody; or an anti-BAFF antibody.

67. The system according to claim 59 or 66, wherein the lupus therapy comprises a first therapy chosen from one or more of:
(i) nonsteroidal anti-inflammatory drug (NSAID);
(ii) an antimalarial drug (e.g., hydroxychloroquine);
(iii) a corticosteroid,
(iv) an immunosuppressant chosen from azathioprine, mycophenolate mofetil, or methotrexate; or
(v) an intravenous immunoglobulin.

68. The system according to claim 59 or 66-67, wherein the lupus therapy comprises a second therapy chosen from one or more of:
(i) an anti-TWEAK antibody;
(ii) an anti-CD40L antibody;
(iii) an anti-CD40 antibody;
(iv) an anti-CD20 antibody;
(v) an anti-interferon antibody;
(vi) rapamycin;
(vii) arsenic trioxide;
(viii) 5-chloro-N-ethyl-4-hydroxy-1-methyl-2-oxo-N-phenyl-1,2-dihydroquinoline-3-carboxamide;
(ix) an anti-BDCA2 antibody; or
(x) an anti-BAFF antibody.

69. The system of any of claims 59-68, wherein the value of disease progression comprises a measure of a combination of a gene signature and a biomarker.

70. The system of any of claims 59-69, wherein the value of disease progression comprises a measure of a combination of two of: a BAFF biomarker (e.g., BAFF mRNA), a TWEAK biomarker (e.g., UTWEAK), or a neutrophil gene signature.

71. The system of any of claims 59-69, wherein the value of disease progression comprises a measure of a combination of all three of: a BAFF biomarker (e.g., BAFF mRNA), a TWEAK biomarker (e.g., UTWEAK), and a neutrophil gene signature.

72. The system of any of claims 59-69, wherein the value of disease progression comprises a measure of a neutrophil gene signature and one or both of: a BAFF biomarker (e.g., BAFF mRNA) or a TWEAK biomarker (e.g., UTWEAK).

73. The system of any of claims 59-69, wherein the value of disease progression comprises a measure of a combination of two or all of: an interferon signature (e.g., IFN-alpha signature), a neutrophil gene signature, or a TWEAK biomarker (e.g., urinary TWEAK or UTWEAK).

74. The system of any of claims 59-69, wherein the value of disease progression comprises a measure of a neutrophil gene signature, and one or both of an interferon signature (e.g., IFN-alpha signature) or a TWEAK biomarker (e.g., urinary TWEAK or UTWEAK).

75. The system of any of claims 59-69, wherein the value of disease progression comprises a measure of an interferon signature (e.g., IFN-alpha signature), and one or both of a neutrophil gene signature or a TWEAK biomarker (e.g., urinary TWEAK or UTWEAK).

76. The system of any of claims 59-69, wherein value of disease progression comprises a measure of a TWEAK biomarker (e.g., urinary TWEAK or UTWEAK), and one or both of an interferon signature (e.g., IFN-alpha signature) or a neutrophil gene signature.

77. The system of any of claims 59-69, wherein the value of disease progression comprises a measure of an IFN signature as an indicator of renal disease activity.
